(19) United States
(12) Patent Application Publication
(10) Pub. No.: US 2022/0098658 A1
(43) Pub. Date: Mar. 31, 2022

(54) METHODS TO MINIMIZE PHOTODAMAGE DURING NUCLEIC ACID AND PEPTIDE SEQUENCING

(71) Applicant: Quantum-Si Incorporated, Guilford, CT (US)

(72) Inventors: Guojun Chen, Sherborn, MA (US); Thomas Raymond Thurston, Guilford, CT (US); An Li, Guilford, CT (US)

(73) Assignee: Quantum-Si Incorporated, Guilford, CT (US)

(21) Appl. No.: 17/481,195

(22) Filed: Sep. 21, 2021

Related U.S. Application Data
(60) Provisional application No. 63/081,014, filed on Sep. 21, 2020.

(51) Int. Cl. C12Q 1/6869; G01N 21/64

(52) U.S. Cl. CPC C12Q 1/6869; G01N 21/648

(57) ABSTRACT

Provided herein are methods and integrated devices for improved sequencing of nucleic acid and peptide biomolecules. The present disclosure relates to improved mechanisms for protecting a luminescent label from photo-induced damage through the use of quenching moieties. Further provided herein are methods for improved immobilization of quenching moieties and other molecules of interest through functionalization with chemical moieties, such as click chemistry handles, capable of participating in cross-linking reactions. Quenching moieties may be immobilized to the surface of a sample well in a sequencing substrate or apparatus in a manner that minimizes or eliminates photobleaching of the labeled molecule. The disclosed methods provide for minimized photodamage, increased sensitivity, accuracy and length of reads during nucleic acid and polypeptide sequencing.

### CROSS-REFERENCE TO RELATED APPLICATIONS

This application claims priority under 35 U.S.C. § 119(e) to U.S. Provisional Patent Application No. 63/081,014, filed Sep. 21, 2020, which is hereby incorporated by reference in its entirety.

### FIELD OF THE DISCLOSURE

The present application is directed generally to methods, compositions, and devices for performing rapid, massively parallel, quantitative analysis of biological and/or chemical samples.

### BACKGROUND

Detection and analysis of biological samples, such as samples containing nucleic acid and polypeptide molecules, may be performed using biological assays (“bioassays”). Bioassays conventionally involve large, expensive laboratory equipment requiring research scientists trained to operate the equipment and perform the bioassays. Moreover, bioassays are conventionally performed in bulk such that a large amount of a particular type of sample is necessary for detection and quantitation.

Bioassays designed for sequencing of nucleic acid and polypeptide molecules are typically performed by labeling these molecules with luminescent markers that emit light of a particular wavelength. The markers are illuminated with excitation light to cause luminescence, and the luminescence is detected with a photodetector to quantify the amount of luminescent light emitted by the markers. Bioassays using luminescent markers conventionally involve expensive laser light sources to illuminate samples and complicated luminescent detection optics and electronics to collect the luminescence from the illuminated samples. There is a need in the art to develop improved sequencing devices and methods that can be operated inexpensively on a desktop or benchtop.

### SUMMARY OF THE DISCLOSURE

Aspects of the technology disclosed herein relate to methods of determining the sequence of a sample containing a polypeptide molecule or a nucleic acid molecule. Further aspects relate to methods and devices for next-generation sequencing of samples, including single-molecule sequencing. Aspects of the disclosure relate to methods and systems for sequencing a polypeptide or a nucleic acid molecule using luminescent molecules such as luminescent labels (e.g., fluorophores). Further aspects relate to use of protecting molecules in conjunction with luminescent molecules to minimize the extent of photo-induced degradation (photodamage or photobleaching) during sequencing.

Systems in accordance with the disclosure permit single-molecule analysis. Exemplary systems include an integrated device (also referred to as a chip) and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a sample containing a single type of biomolecule). In some embodiments, the number of samples within a sample well may be distributed among the sample wells of the integrated device such that some sample wells contain one sample while others contain zero, two or more samples.

In methods and systems in accordance with this disclosure, excitation light is provided to the integrated device from one or more light sources external to the integrated device. Optical components of the integrated device may receive the excitation light from the light source(s), direct the light towards the array of sample wells of the integrated device, and illuminate an illumination region within the sample well. In some embodiments, a sample well may have a configuration that allows for the sample to be retained in proximity to a surface of the sample well, which may ease delivery of excitation light to the sample and detection of emission light from the sample. A labeled biomolecule, or a labeled molecule interacting with the biomolecule, positioned within the illumination region may emit emission light in response to being illuminated by the excitation light. For example, a molecule interacting with the biomolecule in the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. The integrated device may include an optical system for receiving excitation light and directing the excitation light among the sample well array.

Single molecule analyses can demand confinement of a molecule of interest to a target region (or volume) of the sample well for successful detection. Where a population of non-specifically bound molecules are within such a region, it can be challenging to filter out interfering signals during analysis and can diminish accuracy of the results by contributing to the overall statistical analysis. The inherent instabilities and potential toxicities of organic luminescent molecules, or dyes or fluorophores, represent a substantial limitation to advancements in single-molecule sequencing. Fluorophores are highly prone to photobleaching, which reduces the amplitude and duration of the sequencing signal, or “read.” The excitation and subsequent return to a lower-energy state of a fluorophore tends to release free radicals into solution. Some fluorophores may cross over from the excited state to occupy a long-lived triplet state, during which no photons are emitted. Presence of free radicals in the sample well volume may interfere with instrument sensitivity. Thus, photodamage-induced inaccuracies represent a bottleneck in sequencing performance.

It is therefore often desirable to protect a luminescent label from photo-induced damage in order to preserve proper functionality and overall activity, for the sake of optimizing readout parameters such as read length and accuracy. Existing methods to remove free radicals have involved the addition of anti-bleaching reagent mixture (often in ethanol) to the sample well. The success of these methods is limited by the diffusion of these reagents away from the fluorophores undergoing photobleaching (a phenomenon sometimes referred to as diffusion-based anti-bleaching). In view of target volumes in sample wells for single-molecule applications of less than an attoliter (1×10⁻¹⁸ L, or (100 nm)³), diffusion from the active site is counterproductive. Consequently, there is a need in the art for the development of improved methods that achieve fluorophore photostabilization, and in particular, positional stabilization by protecting molecules such as quenching moieties.

The methods and integrated devices described herein provide protecting functionality that eliminates or minimizes photo-induced damage from luminescent labels. In some embodiments, these methods provide protecting functionality that minimizes photodamage to a labeled biomolecule—and the sensitivity of the system as a whole—that otherwise results from a luminescent label releasing free radicals into solution. In some embodiments, these methods and devices provide protecting functionality that minimizes photodamage to an enzyme (e.g., a polymerase, a peptidase). In certain instances, for example, these methods and devices minimize photodamage to an active site of an enzyme (e.g., a polymerase, a peptidase). In nucleic acid sequencing, for example, the activity of the nucleic acid polymerizing enzyme can be determinative of the robustness of the sequencing results. That is because many next-generation applications are based on the “sequencing by synthesis” principle, in which the sequencing is performed by detecting the nucleotide incorporated by a DNA polymerase. Many next-generation sequencing by synthesis applications provide for sequencing a single strand of a nucleic acid of interest (e.g., ssDNA) by synthesizing a complementary strand along it, one base pair at a time, and detecting which base was added at each step.

The disclosed methods and devices are based, at least in part, on the use of quenching moieties as protecting molecules. Quenching moieties, such as triplet state quenching moieties, “scavenge” free radicals in solution, enabling the fluorophore to re-enter the ground energy state following excitation.

The methods and integrated devices described herein further provide improved strategies for functionalization of a surface (e.g., bottom surface) of a sample well with a moiety capable of coupling (or cross-linking) together molecules of interest. Such a coupling moiety may be capable of coupling a biomolecule (e.g., a nucleic acid and/or a polymerase, or a peptide) to another molecule or to a surface of the sample well. In some aspects, the methods and devices described herein provide for the substantial immobilization of one or more of a biomolecule, quenching moiety, and coupling moiety in close proximity to one another. The methods may achieve minimization or elimination of photodamage by, for instance, reducing the extent of diffusion between these components within a volume of the sample well.

In some aspects, the disclosure provides methods and compositions for determining nucleotide sequence information from nucleic acid biomolecules (e.g., for sequencing a polynucleotide). In some embodiments, nucleotide sequence information can be determined for single nucleic acid biomolecules. The sequencing methods of the disclosure may comprise “sequencing by synthesis” assays. The sequencing methods of the disclosure may comprise time-course (or time domain) measurement assays.

In some embodiments, the nucleic acid sequencing methods of the disclosure comprise detecting a time-course of incorporation of a series of labeled nucleotides. Nucleotides may be conjugated to the same type, or different types, of luminescent label. Application of excitation light excites labeled nucleotides within an illumination region. In some embodiments, the nucleic acid sequencing methods can be used to identify a series of nucleotides that are incorporated into a template-dependent nucleic acid sequencing reaction product synthesized by a polymerizing enzyme (e.g., DNA polymerase). Thus, in some embodiments, described herein are methods of nucleic acid sequencing that comprise steps of: (i) exposing a complex in a target volume in a sample well to one or more labeled nucleotides, the complex comprising a target nucleic acid present in a sample, a primer, and a DNA polymerase; (ii) directing a series of pulses of excitation light towards the target volume; (iii) detecting a plurality of emitted photons from the one or more labeled nucleotides during sequential incorporation into a nucleic acid comprising the primer; and (iv) identifying the sequence of incorporated nucleotides by determining one or more characteristics of the emitted photons. These characteristics may be selected from luminescent lifetime, retention time, luminescent intensity, luminescent wavelength, pulse duration, and/or interpulse duration. In some embodiments, the characteristic is luminescent lifetime.

In other embodiments, a characteristic (e.g., a luminescent lifetime) is produced only when a nucleic acid polymerase, such as a DNA polymerase, successfully incorporates a nucleotide that complements the first unpaired base of the target nucleic acid.

In other aspects, the disclosure provides methods and compositions for determining amino acid sequence information from polypeptides (e.g., for sequencing one or more polypeptides). In some embodiments, amino acid sequence information can be determined for single polypeptide biomolecules. In some embodiments, the relative position of two or more amino acids in a polypeptide is determined, for example for a single polypeptide molecule. Single-molecule polypeptide sequencing may occur by stepwise degradation, in contrast to DNA sequencing by synthesis.

According to some aspects, polypeptide sequencing methods of the disclosure comprise obtaining data during a degradation process of a polypeptide, analyzing the data to determine portions of the data corresponding to amino acids that are sequentially exposed at a terminus of the polypeptide during the degradation process, and outputting an amino acid sequence representative of the polypeptide. In some embodiments, the data is indicative of amino acid identity at the terminus of the polypeptide during the degradation process. In some embodiments, the data is indicative of a signal produced by one or more amino acid recognition molecules binding to different types of terminal amino acids at the terminus during the degradation process. In some embodiments, the data is indicative of a luminescent signal generated during the degradation process. In some embodiments, the data is indicative of an electrical signal generated during the degradation process. In some embodiments, analyzing the data further comprises detecting a series of cleavage events and determining the portions of the data between successive cleavage events. In some embodiments, analyzing the data further comprises determining a type of amino acid for each of the individual portions. In some embodiments, each of the individual portions comprises a pulse pattern (e.g., a characteristic pattern), and analyzing the data further comprises determining a type of amino acid for one or more of the portions based on its respective pulse pattern. In some embodiments, determining the type of amino acid further comprises identifying an amount of time within a portion when the data is above a threshold value and comparing the amount of time to a duration of time for the portion. In some embodiments, determining the type of amino acid further comprises identifying at least one pulse duration for each of the one or more portions. In some embodiments, determining the type of amino acid further comprises identifying at least one interpulse duration for each of the one or more portions. In some embodiments, the amino acid sequence includes a series of amino acids corresponding to the portions.

In some embodiments, the polypeptide sequence methods of the disclosure comprise contacting a single polypeptide molecule with one or more terminal amino acid recognition molecules. In some embodiments, the methods further comprise detecting a series of signal pulses indicative of association of the one or more terminal amino acid recognition molecules with successive amino acids exposed at a terminus of the single polypeptide molecule while it is being degraded, thereby obtaining sequence information about the single polypeptide molecule. In some embodiments, the amino acid sequence of most or all of the single polypeptide molecule is determined. In some embodiments, the series of signal pulses is a series of real-time signal pulses. In some embodiments, association of the one or more terminal amino acid recognition molecules with each type of amino acid exposed at the terminus produces a characteristic pattern in the series of signal pulses that is different from other types of amino acids exposed at the terminus. In some embodiments, a signal pulse of the characteristic pattern corresponds to an individual association event between a terminal amino acid recognition molecule and an amino acid exposed at the terminus. In some embodiments, the characteristic pattern corresponds to a series of reversible terminal amino acid recognition molecule binding interactions with the amino acid exposed at the terminus of the single polypeptide molecule. In some embodiments, the characteristic pattern is indicative of the amino acid exposed at the terminus of the single polypeptide molecule and an amino acid at a contiguous position (e.g., amino acids of the same type or different types).

Accordingly, in some aspects, the disclosure provides methods of sequencing a biomolecule, the method comprising i) functionalizing a surface of a sample well with a coupling moiety, ii) coupling the coupling moiety with a copolymer comprising a quenching moiety; iii) contacting the sample well with the sample; and iv) applying an excitation signal. In some embodiments, the surface of the sample well is the bottom surface of the well. In some embodiments, the methods further comprise v) determining a discriminable luminescent property of the biomolecule or a molecule interacting with the biomolecule in the sample; and vi) identifying the biomolecule or the molecule interacting with the biomolecule based on its luminescent properties. In some embodiments, the luminescent property is selected from a luminescent lifetime, a retention time, a luminescent intensity, a luminescent wavelength, a pulse duration, an interpulse duration, and/or a combination thereof. In particular embodiments, the luminescent property comprises luminescent lifetime and/or luminescent intensity. In various embodiments, the quenching moiety is a triplet state quenching moiety.

In some aspects, the disclosure provides methods of sequencing a biomolecule, the method comprising i) first contacting the sample well with the sample; and subsequently ii) functionalizing a surface of a sample well with a coupling moiety, iii) coupling the coupling moiety with a copolymer comprising a quenching moiety; and iv) applying an excitation signal.

In some aspects, the disclosure provides nucleic acid sequencing methods that make use of the described coupling moieties, copolymers, and functionalization techniques. Accordingly, in some embodiments, the disclosure provides methods of sequencing a nucleic acid biomolecule in a sample, the method comprising: functionalizing a bottom surface of a sample well with a coupling moiety; coupling the coupling moiety with a copolymer comprising a triplet state quenching moiety; providing a sample comprising the nucleic acid biomolecule, a primer complementary to the nucleic acid biomolecule, a nucleic acid polymerase, and luminescently labeled nucleotides; applying an excitation signal; determining a luminescent lifetime, a retention time, a luminescent intensity, a luminescent wavelength, a pulse duration, and/or an interpulse duration of the luminescently labeled nucleotides during incorporation into a growing nucleic acid strand complementary to the nucleic acid biomolecule; and identifying a nucleotide sequence of at least a portion of the biomolecule based on one of these characteristics of the luminescently labeled nucleotides. In some embodiments, the step of providing a sample, primer, nucleic acid polymerase, and luminescently labeled nucleotides precedes the step of functionalizing a bottom surface of a sample well with a coupling moiety.

In some aspects, the disclosure provides polypeptide sequencing methods that make use of the described coupling moieties, copolymers, and functionalization techniques. Accordingly, in some embodiments, the disclosure provides methods of sequencing a biomolecule (e.g., a polypeptide biomolecule) in a sample, the method comprising: i) functionalizing a bottom surface of a sample well with a coupling moiety; ii) coupling the coupling moiety with a copolymer comprising a triplet state quenching moiety; iii) contacting the sample well with the sample and luminescently labeled molecules that interact with the biomolecule; iv) applying an excitation signal; v) determining a luminescent lifetime, a retention time, a luminescent intensity, a luminescent wavelength, a pulse duration, and/or an interpulse duration of the luminescently labeled molecules; and vi) identifying an amino acid sequence of at least a portion of the biomolecule based on one of these characteristics of the luminescently labeled molecules.

In some embodiments, the disclosure provides methods of sequencing a biomolecule (e.g., a polypeptide biomolecule) in a sample, the method comprising: i) first contacting the sample well with the sample and luminescently labeled molecules that interact with the biomolecule; and subsequently ii) functionalizing a bottom surface of a sample well with a coupling moiety, iii) coupling the coupling moiety with a copolymer comprising a triplet state quenching moiety, iv) applying an excitation signal, v) determining a luminescent lifetime, a retention time, a luminescent intensity, a luminescent wavelength, a pulse duration, and/or an interpulse duration of the luminescently labeled molecules, and vi) identifying an amino acid sequence of at least a portion of the biomolecule based on one of these characteristics of the luminescently labeled molecules.

In some embodiments of the disclosure, the disclosed sequencing methods comprise identifying a nucleotide or amino acid sequence of the biomolecule based on the luminescent lifetime and/or the luminescent intensity characteristics of labeled molecules that interact with the biomolecule of interest. Exemplary labeled molecules that interact with a biomolecule of interest include nucleotides, which interact with a nucleic acid biomolecule, and amino acid recognition molecules, which interact with a peptide biomolecule.

In some aspects, the disclosure provides methods of selectively functionalizing a surface of a sample well that involve contacting a sample well surface with a copolymer that preferentially binds a coating layer on the surface. In some embodiments, the sample well is contacted with the copolymer, e.g., in an amount sufficient to form an overlay over the surface. In some embodiments, the methods further involve contacting the sample well with a functionalizing agent that preferentially binds the surface to generate a functionalized surface, wherein the functionalizing agent comprises a coupling moiety.

In some embodiments, the coupling moiety of the functionalizing agent bound to the sample well surface comprises a biotin moiety, an avidin protein, a streptavidin protein, a biotin-streptavidin complex, an azide moiety, an alkyne moiety, a ketone moiety, or a hydroxylamine moiety. In some embodiments, the coupling moiety comprises a lectin protein or a SNAP-TAG® (a self-labeling protein tag). In particular embodiments, the coupling moiety is selected from a biotin moiety or an azide moiety. In some embodiments, the coupling moiety of the functionalizing agent bound to the sample well surface comprises an amine group, an azido group, a carboxyl group, a hydroxyl group, an alkyl group, an alkyne group, or a sulfhydryl group.

Aspects of the disclosure are useful for sequencing biological polymers, such as nucleic acids and proteins. In some aspects, the single molecule in the sample is a nucleic acid. In some embodiments, the nucleic acid is single-stranded. In some embodiments, the nucleic acid is double-stranded.

In some aspects, the single molecule in the sample is a polypeptide. In some aspects, methods of immobilizing a polypeptide to a surface of a sample well are provided. In some embodiments, the surface is functionalized with a complementary functional moiety configured for attachment (e.g., covalent or non-covalent attachment) to a functionalized terminal end of a peptide. In some embodiments, confining a single peptide per sample well is advantageous for single molecule detection methods, e.g., single molecule peptide sequencing.

In some embodiments, the copolymer comprises a grafted copolymer in which a quenching moiety has been grafted (or conjugated) onto a graft of two or more polymers. In In particular embodiments, the two or more polymers of the grafted copolymer comprise poly-L-lysine (PLL) and polyethylene glycol (PEG). In some embodiments, the copolymer comprises a non-PLL polymer. In some embodiments, the copolymer comprises a non-PEG polymer. In some embodiments, the copolymer comprises a graft of PLL, PEG, and a third polymer. In some embodiments, the quenching moiety of the copolymer comprises at least one triplet state quencher. In some embodiments, the quenching moiety is selected from triplet state quenchers trolox, 4-nitrobenzyl alcohol (NBA), and cyclooctatetraene (COT). In certain embodiments, the quenching moiety is trolox (6-hydroxy-2,5,7,8-tetramethylchroman-2-carboxylic acid), a water-soluble analog of vitamin E. In some embodiments, the quenching moiety is a quinone derivative of trolox, trolox quinone, which is produced by oxidizing trolox. In some embodiments, the quenching moiety is NBA. In some embodiments, the quenching moiety is COT.

In some aspects, the present disclosure provides a method of functionalization of a sample well surface that comprises contacting the surface with a moiety comprising a click chemistry handle. In some embodiments, the click chemistry handle (e.g., a copolymer comprising a click chemistry handle) may be further functionalized to a coupling moiety.

In some aspects, the disclosed methods further comprise a step of functionalizing the surface of the sample well with a second coupling moiety. In some embodiments, the biomolecule binds this second coupling moiety. In some embodiments, the biomolecule (e.g., a peptide or nucleic acid) is contacted with a copolymer (e.g., comprising a click chemistry handle) in a manner sufficient to achieve binding to the second coupling moiety. In some embodiments, the biomolecule is contacted with the first coupling moiety and/or the second coupling moiety. In some embodiments, the second coupling moiety comprises a biotin moiety, an avidin protein, a streptavidin protein, an azide moiety, an alkyne moiety, a ketone moiety, and/or a hydroxylamine moiety. In particular embodiments, the second coupling moiety comprises a biotin moiety. In some embodiments, the second coupling moiety comprises an azide moiety. In some embodiments, the second coupling moiety comprises an alkyne moiety. Embodiments of the methods may comprise the coupling of a second copolymer to the second coupling moiety. In certain embodiments, the second copolymer is a click chemistry handle. In some embodiments, the second copolymer comprises a graft of poly-L-lysine (PLL) and polethylene glycol (PEG).

Aspects of the present application provide methods for delivering an excitation energy to a molecule (e.g., a luminescently labeled nucleoside polyphosphate or amino acid recognition molecule) to be identified and detecting emitted photons after the excitation. In certain embodiments, detecting comprises recording for each detected luminescence the time duration between the luminescence and the prior pulse of excitation energy. In certain embodiments, detecting comprises recording for each of a plurality of detected luminescences the time duration between the luminescence and the prior pulse of excitation energy. In certain embodiments, a plurality of pulses of excitation energy are delivered. The luminescent marker (e.g., the one or more luminescent labels) of the molecule to be identified may be excited by each pulse or a portion of the pulses. In certain embodiments, a plurality of luminescences are detected by one or more sensors. The luminescent marker of the molecule to be identified may emit luminescence after each excitation or a portion of the excitations. The fraction of excitation events that result in a luminescence is based on the luminescence quantum yield of the marker. In some embodiments, increasing the number of luminescent labels can increase the quantum yield (e.g., increase the number of luminescence emissions). Additionally, not all luminescences emitted by a marker will be detected, for example, some luminescences will be directed away from the sensors. In certain embodiments, the excitation energy or energies are selected based on the luminescent properties of the luminescent markers, including the absorption spectra and wavelengths at which a marker emits photons after excitation in a given spectral range.

In some embodiments, the detectable signals are optical signals. In some embodiments, the optical signals are luminescent signals. In some embodiments, determining the timing and/or frequency of the measured detectable signals comprises (i) receiving said detectable signals at one or more sensors; and (ii) selectively directing charge carriers of a plurality of charge carriers produced in response to said detectable signals received at said one or more sensors into at least one charge carrier storage region (or bin) based upon times at which said charge carriers are produced.

In some embodiments, the timing and/or frequency of said measured detectable signals comprise measurements of decay lifetimes. In some embodiments, the timing and/or frequency of the measured detectable signals comprise measurements of arrival times of the detectable signals at one or more sensors that detect the detectable signals. In some embodiments, the method further comprises segregating charge carriers produced by the detectable signals into bins associated with the one or more sensors based on the arrival times of the detectable signals. In some embodiments, the timing and/or frequency of the measured detectable signals are non-spectral measurements.

### BRIEF DESCRIPTIONS OF THE DRAWINGS

The skilled artisan will understand that the figures, described herein, are for illustration purposes only. It is to be understood that in some instances various aspects of the disclosure may be shown exaggerated or enlarged to facilitate an understanding of the disclosure. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way. The features and advantages of the present disclosure will become more apparent from the detailed description set forth below when taken in conjunction with the drawings. As is apparent from the detailed description, the examples depicted in the figures (e.g., FIGS.) and Further Described for the Purpose of Illustration Throughout the disclosure describe non-limiting embodiments, and in some cases may simplify certain processes or omit features or steps for the purpose of clearer illustration.

FIG. 1 is a schematic of an exemplary sample well containing various components for nucleic acid sequencing.

FIG. 2 shows an exemplary experiment of nucleic acid sequencing for four stages; (A) before incorporation of a luminescently labeled nucleotide; (B) a first incorporation event; (C) a period between the first and second incorporation events; and (D) a second incorporation event; along with corresponding examples of raw and processed data during stages (A)-(D).

FIG. 3 is a schematic that shows a charge carrier confinement region of a pixel of an exemplary integrated photodetector.

FIG. 4 is a diagram of an exemplary microchip architecture of a next-generation sequencing instrument.

FIG. 5 shows example workflows for surface modification in accordance with the disclosure. This figure shows a workflow for preparing a selectively functionalized surface portion of a sample well and a workflow for coupling a molecule of interest to a functionalized surface. At bottom, an example of a sample well having modified surfaces is shown.

FIGS. 6A-6B show an exemplary surface functionalization method, according to embodiments of the disclosure. FIG. 6A shows the chemical structure of an exemplary graft of poly-L-lysine (PLL) and polyethylene glycol (PEG) (“PLL-PEG”) and a list of several grafted copolymers of a coupling moiety, PLL-PEG, and a quenching moiety. FIG. 6B shows a sample well nanoaperture comprising a bottom surface that has been functionalized with a) a streptavidin coupling moiety and an exemplary graft copolymer of FIG. 6A, and b) a biotin coupling moiety and a DNA polymerase. Abbreviations, “N₃”=azide moiety; “TXV”=trolox; “NBA”=4-nitrobenzyl alcohol; “COT”=cyclooctatetraene.

FIG. 7A depicts the chemical synthesis of an exemplary high-density grafted copolymer comprising biotin-PEG-PLL grafted with trolox (TXV). FIG. 7B shows the chemical structures of trolox, NBA and COT.

FIG. 8 illustrates exemplary DNA and peptide sequencing workflows that make use of the biotin-containing grafted copolymers of the disclosure. The workflows depicted make use of coupling moieties (“terminals”) selected from a methyl ether, a biotin, and a biotin-streptavidin complex. Functional groups are represented by shapes according to the “Annotation.” The DNA or peptide biomolecule is labeled with “E”; and the biotin-containing grafted copolymer is labeled with “F.”

FIG. 9 illustrates exemplary DNA and peptide sequencing workflows that make use of the azide-containing grafted copolymers of the disclosure. The workflows depicted make use of coupling moieties selected from a biotin, an azide, and an alkyne group. Functional groups are represented by shapes according to the “Annotation” legend, as described above for FIG. 8.

FIG. 10 depicts the accuracy and read length results of a DNA sequencing experiment performed according to embodiments of this disclosure. In this assay, a DNA molecule to be sequenced, a DNA polymerase, and a graft copolymer of PLL-PEG containing a trolox quenching moiety were combined in a sample well. Sequencing read length and accuracy were measured and charted in the graphs shown. This sequencing experiment was performed on a complementary metal oxide semiconductor (CMOS) chip.

FIG. 11 depicts an exemplary DNA sequencing experiment, wherein the luminescent properties of a luminescently-labeled nucleotide are used to identify the base being incorporated into the sequencing reaction. The sequencing experiment is performed on a CMOS chip.

FIGS. 12A and 12B depict the accuracy (FIG. 12A), and the pulse duration and read length (FIG. 12B) results of another DNA sequencing CMOS chip experiment performed according to embodiments of this disclosure, in which a DNA molecule to be sequenced, a DNA polymerase, and a graft copolymer of PLL-PEG containing a cyclooctatetraene (COT) quenching moiety were combined in a sample well.

### DETAILED DESCRIPTION

The present disclosure provides methods and integrated devices for improved sequencing of nucleic acid and peptide biomolecules. The present disclosure relates to improved mechanisms for protection of luminescently labeled molecules from photobleaching by reactive species through the use of quenching moieties. The present disclosure further relates to the immobilization of quenching moieties to the surface of a sample well in a sequencing substrate or apparatus. In some aspects, these mechanisms confine a protecting element and a biomolecule in very close proximity to one another, so as to minimize or eliminate photobleaching of labeled molecules, such as labeled biomolecules and labeled molecules interacting with the biomolecule. In particular aspects, the disclosed immobilization mechanisms comprise high-density grafted copolymers. Previous techniques for surface modification had used liquid phosphonic acid derivatives to passivate metallic surfaces to achieve functionalization. The inventors surprisingly found that the grafting of polymer reagents onto one another and subsequently onto one or more quenching moieties generated coupling moieties (copolymers) that provided stronger biomolecule functionalization to the sample well surface and provided enhanced nucleic acid and polypeptide sequencing accuracy. Based on these findings, the inventors developed various high-density grafted copolymers for use in confining biomolecules and quenching moieties within the same target volume (on the order of an attoliter) of a sample well and minimizing diffusion of these molecules from one another. The degree of photobleaching may be measured by any method known in the art, including bulk bleaching analyses.

Provided herein are methods of identifying a single molecule in a sample comprising a biomolecule (e.g., a nucleic acid, a polymerase, or a polypeptide). In various aspects, the disclosed methods of sequencing a nucleic acid involve determining the luminescent lifetimes, and optionally luminescence intensities, of a series of luminescently labeled nucleotides incorporated during a nucleic acid sequencing reaction. In some aspects, the disclosed methods of sequencing a polypeptide involve determining the luminescent lifetimes, and optionally luminescence intensities, of a series of luminescently labeled amino acid recognition molecules interacting with terminal amino acids during a polypeptide sequencing reaction. The disclosed methods provide for minimized photodamage; and increased sensitivity, accuracy and length of reads during nucleic acid and polypeptide sequencing.

### Sample Wells and Integrated Devices

Accordingly, in some aspects, the disclosure provides an integrated device comprising a substrate comprising a sample well having a metal oxide surface and a silica surface. In some embodiments, the integrated device further comprises a coating layer on the metal oxide surface formed by an amphipathic reagent that comprises a hydrophilic head group and a hydrophobic tail group. In some embodiments, the integrated device further comprises a functionalizing agent bound to the silica surface, wherein the functionalizing agent comprises a coupling moiety. In some embodiments, the substrate comprises an array of sample wells, each sample well having a metal oxide surface and a silica surface. In some embodiments, the array is a microfabricated microarray. In some embodiments, the sample well comprises a top aperture formed at a surface of the substrate and a bottom surface distal to the surface of the substrate. In some embodiments, the bottom surface is comprised by the silica surface.

As used herein, in some embodiments, a “surface” refers to a surface of a substrate or solid support. In some embodiments, a substrate refers to a material, layer, or other structure having a surface, such as a receiving surface, that is capable of supporting a deposited material, such as a layer or a coating described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one-dimensional array is an array having one column or row of elements in the first dimension and a plurality of columns or rows in the second dimension. The number of columns or rows in the first and second dimensions may or may not be the same. In some embodiments, the array may include, for example, 102, 103, 104, 105, 106, or 107 sample wells. In some embodiments, the array is a microfabricated microarray. In some embodiments, the substrate contains 128,000 sample wells. In some embodiments, the surface of any of the sample wells in the array contains a metal oxide.

In some embodiments, the sample well occupies a volume or space defined by an opening formed at a surface of an integrated device which extends through a first layer and into a second layer of the integrated device to a bottom surface distal to the opening. In some embodiments, the exposed surfaces of the first layer and second layer disposed between the opening and the bottom surface of the sample well may be referred to as sidewalls which further define the volume or space occupied by the sample well. In some aspects, the present disclosure provides a method of selective functionalization of a biomolecule (e.g., a peptide or nucleic acid) to a surface of a sample well, such as the bottom surface of a well. In some embodiments, the first layer is a metal cladding layer. In some embodiments, the metal cladding layer comprises one or more types of metals (e.g., aluminum, titanium, zirconium, iron, tin, tantalum, etc.). In some embodiments, the exposed surface portions of the first layer comprise a metal oxide. In some embodiments, the second layer is a transparent material or glass. In some embodiments, the exposed surface portions of the second layer comprise fused silica or silicon dioxide. In some embodiments, the sidewalls of the sample well are composed of at least a portion of each of the exposed surface portions of the first and second layers. In some embodiments, the bottom surface of the sample well comprises silica. In some embodiments, at least a portion of the sidewalls adjacent to the bottom surface comprises silica.

In some embodiments, a sample well comprises an immobilization region that may be a discrete region of a surface of a substrate that binds a molecule of interest, such as a bottom surface of a sample well having a polypeptide or a nucleic acid coupled to such surface. In some embodiments, sample wells comprise hollows or wells having defined shapes and volumes which are manufactured into a substrate or device. Sample wells can be fabricated using techniques described in the art, for example, as disclosed in U.S. application Ser. No. 16/555,902, which is incorporated herein by reference in its entirety.

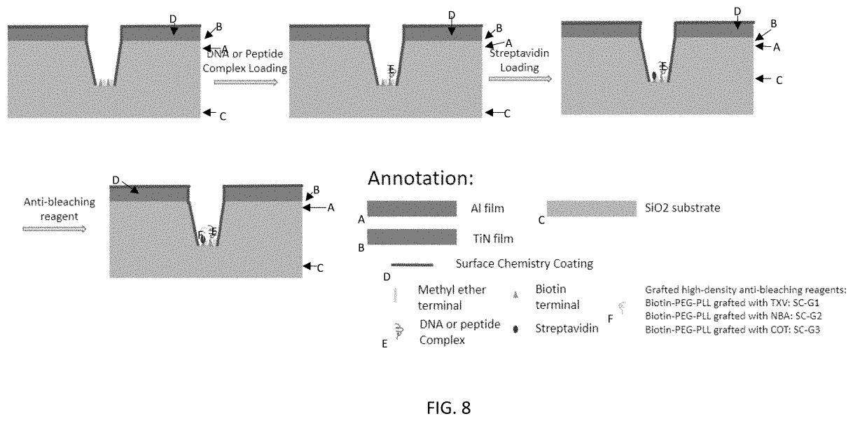

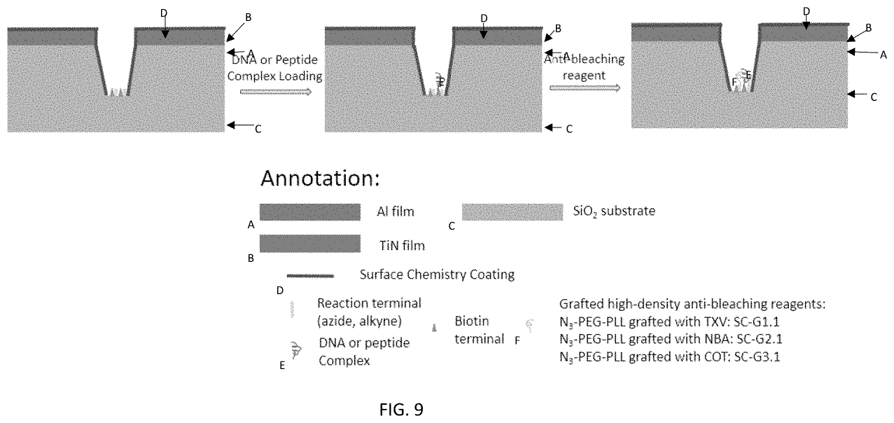

In some embodiments, the sample well is formed by a bottom surface comprising a non-metallic layer and side wall surfaces comprising a metallic layer. In some embodiments, the non-metallic layer comprises a transparent layer (e.g., glass, silica). In some embodiments, the metallic layer comprises a metal oxide surface (e.g., titanium dioxide). In some embodiments, the metallic layer comprises a passivation coating (e.g., a phosphorus-containing layer, such as an organophosphonate layer). The bottom surface may comprise a non-metallic layer that comprises a functional moiety. In some embodiments, the sample well comprises a top surface that contains an aluminum film, a titanium nitride (tinite, TiN) film, or both (see FIGS. 8 and 9, annotated with “A” and “B”). The top surface may further comprise a selective surface chemistry coating, such as a coating known in the art (see coating annotated with “D” in FIGS. 8 and 9).

As used herein, an “integrated device” is a device configured to perform single-molecule analysis and capable of interfacing with a base instrument. In some embodiments, an integrated device may comprise one or more sample wells and/or sensors. In some embodiments, an integrated device may be capable of interfacing with a base instrument that emits or detects light, such as a next-generation sequencing instrument. In such embodiments, the integrated device may comprise one or more sample wells, each of which includes a waveguide. The “integrated device” may be referred to herein as a microchip (or “chip”), such as a CMOS chip. An exemplary integrated device is a CMOS chip containing an array of pixels, where individual pixels include a sample well and photodetector.

The disclosed methods of detection are adapted to be performed on a solid substrate containing a plurality of sample wells. In some embodiments, this substrate is adapted to be used on, or with, a microchip adapted for use with a diagnostic test. In some embodiments, the substrate is a layer of a metal oxide chip, such as a CMOS chip. In some embodiments, the substrate is a layer of a CMOS chip adapted for use with an instrument, such as the Platinum next-generation sequencing instrument (Quantum-Si).

The methods provided herein may be associated with a system in which an instrument into which the integrated device is inserted and cooperates includes at least one laser light source, and the integrated device includes waveguides for directing light and pixels having sample wells and detection regions. However, it will be understood that these methods are not limited to use in conjunction with an integrated device. Other instrument designs and integrated device designs are envisioned, including ones where the light sources are not located within the instrument, and/or where additional optical components are located within the instrument (e.g., “off-chip”) rather than the integrated device (e.g., “on-chip”).

Emission light emitted from one or more sample wells (e.g., at least two sample wells, in some embodiments) may be detected by one or more photodetectors within a pixel of the integrated device. As described above, the integrated device may be configured to have multiple pixels (e.g., an array of pixels) and, thus, may have multiple sample wells and corresponding photodetectors. Characteristics of this emission light may provide an indication for identifying the label associated with the emission light. Such characteristics may include any suitable type of characteristic, including an arrival time of photons detected by the photodetector, an amount of photons accumulated over time by a photodetector, and/or a distribution of photons across two or more photodetectors. In some embodiments, a photodetector may have a configuration that allows for detection of one or more characteristics associated with the emission light, such as timing characteristics (e.g., luminescent lifetime, pulse duration, interpulse duration), wavelength, and/or intensity. As one example, one or more photodetectors may detect a distribution of photon arrival times after a pulse of excitation light propagates through the integrated device, and the distribution of arrival times may provide an indication of a timing characteristic of the label's emission light (e.g., a proxy for luminescent lifetime, pulse duration, and/or interpulse duration). In various embodiments, the label is conjugated to a molecule interacting with a biomolecule in a sample of interest, such as a nucleic acid or protein to be sequenced.

In some aspects, provided herein is an integrated device comprising: i) a substrate comprising an array of sample wells having a metal oxide surface; ii) a functionalizing agent bound to the metal oxide surface, wherein the functionalizing agent comprises a coupling moiety; and ii) a copolymer comprising a triplet state quenching moiety. The coupling moiety may comprise any of the coupling moieties described herein. In certain embodiments, the coupling moiety comprises a biotin moiety, an avidin protein, a streptavidin protein, an azide moiety, an alkyne moiety, a ketone moiety, or a hydroxylamine moiety. In particular embodiments, the coupling moiety comprises a biotin moiety. In some embodiments, the integrated device is configured to interface with a next-generation sequencing instrument, such as a benchtop NGS instrument.

An integrated device of the type described herein may comprise one or more sample wells configured to receive molecules of interest therein. In some embodiments, a sample well receives a molecule of interest that may be disposed on a surface of the sample well, such as a bottom surface. In some embodiments, a sample well is formed within an integrated device, wherein the bottom surface of the sample well is distal to the surface of the integrated device into which it is formed. In some embodiments, the bottom surface on which the molecule of interest is to be disposed may have a distance from a waveguide that is configured to excite the molecule of interest with a desired level of excitation energy. In some embodiments, the sample well may be positioned, with respect to a waveguide, such that an evanescent field of an optical mode propagating along the waveguide overlaps with the molecule of interest.

A sample well may have a top opening at the surface of an integrated device through which a molecule of interest may be placed in the sample well. The size of the top opening may depend on different factors, such as the size of the molecules of interest (e.g., sequencing templates, polymerizing enzymes) in the sample being loaded. In some embodiments, the size of the top opening may depend upon the instrument or apparatus in which integrated device comprising the sample well is being utilized. For example, in devices that detect light from within the sample well, background signals may result from stray light. When a molecule of interest is disposed in the sample well and excited with excitation energy, background signals may cause undesired fluctuations in the emission energy, thus making the measurement noisy. To limit such fluctuations, the size of the top opening may be configured to block at least a portion of the background signals.

The volume of a sample well may be between about 10⁻²¹ liters and about 10⁻¹⁵ liters (a femtoliter) in some embodiments. In particular embodiments, the target volume in a sample well is about 1×10⁻²⁰ L. In certain embodiments, the target volume is 1.2×10⁻²⁰ L. Because the sample well has a small volume, detection of single-sample events (e.g., single-molecule events) may be possible even though molecules of interest may be concentrated in an examined specimen at concentrations that are similar to those found in natural environments. For example, micromolar concentrations of the molecule of interest may be present in a specimen that is placed in contact with the integrated device, but at the pixel level only about one molecule of interest (or single molecule event) may be within a sample well at any given time.

Statistically, some sample wells may contain no molecules of interest and some may contain more than one molecule of interest. However, an appreciable number of sample wells may contain a single molecule of interest (e.g., at least 30% in some embodiments), so that single-molecule analysis can be carried out in parallel for a large number of sample wells. Because single-molecule or single-sample events may be analyzed at each sample well, the integrated device makes it possible to detect individual events that may otherwise go unnoticed in ensemble averages.

### Surface Functionalization or Immobilization

In certain embodiments, techniques described herein can be used to functionalize (or immobilize, or confine) a molecule of interest to a desired region of a sample well. In some embodiments, the desired region may be referred to as a “target volume”, a “detection region,” or a “nanoaperture.”

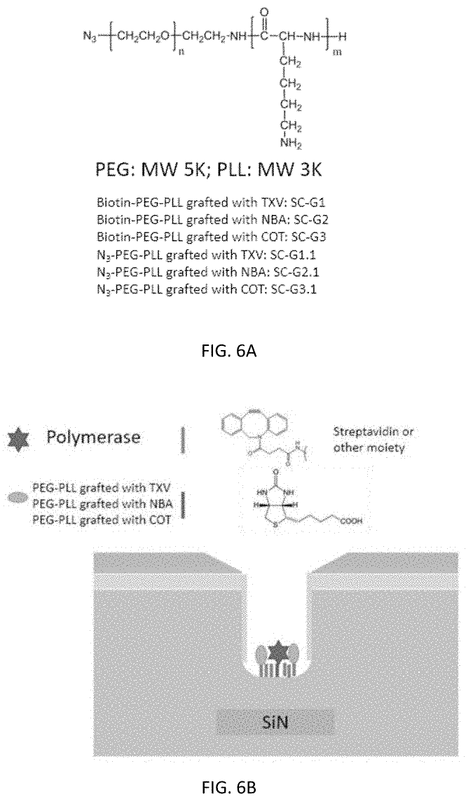

In embodiments when one or more molecules or complexes (e.g., a DNA complex or a peptide complex) is immobilized on the bottom surface of the sample well, it may be desirable to functionalize the bottom surface to allow for attachment of one or more molecules or complexes. In certain embodiments, the bottom surface comprises a transparent glass. In certain embodiments, the bottom surface comprises fused silica or silicon dioxide. In some embodiments, the bottom surface is functionalized with a silane. In some embodiments, the bottom surface is functionalized with an ionically charged polymer or copolymer. In some embodiments, the ionically charged polymer comprises poly(lysine). In some embodiments, the bottom surface is functionalized with a graft of poly-L-lysine (PLL) and polyethylene glycol (PEG), such as poly(lysine)-graft-poly(ethylene glycol) (see FIGS. 6A, 8 and 9). In some embodiments, the bottom surface is functionalized with a biotin-streptavidin complex.

In some embodiments, the bottom surface is functionalized with a silane comprising an alkyl chain. In some embodiments, the bottom surface is functionalized with a silane comprising an optionally substituted alkyl chain. In some embodiments, the bottom surface is functionalized with a silane comprising a poly(ethylene glycol) chain. In some embodiments, the bottom surface is functionalized with a silane comprising a coupling moiety. For example, the coupling moiety may comprise chemical moieties, such as amine groups, carboxyl groups, hydroxyl groups, sulfhydryl groups, metals, chelators, and the like. Alternatively, the coupling moiety may include specific binding elements, such as biotin, avidin, streptavidin, neutravidin, lectins, SNAP-TAGs® (self-labeling protein tags), associative or binding peptides or proteins, antibodies or antibody fragments, nucleic acids or nucleic acid analogs, or the like. The coupling moiety may comprise a methyl ether group. Additionally, or alternatively, an intermediate binding agent and/or copolymer may be used to couple an additional group that is used to couple or bind with the molecule of interest, which may, in some cases, include both chemical functional groups and specific binding elements. By way of example, a coupling moiety, e.g., biotin, may be deposited upon a substrate surface and may be selectively activated in a given area. An intermediate binding agent, e.g., streptavidin, may then be coupled to the biotin coupling moiety. The molecule of interest, which in an exemplary workflow may itself be biotinylated, may then be coupled to the streptavidin. In other embodiments, a grafted copolymer (e.g., a PEG-PLL copolymer) comprising a quenching moiety (e.g., trolox) is coupled to the streptavidin (see FIGS. 6B and 8).

The chemical structure for biotin is reproduced below:

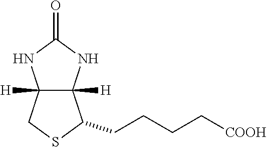 [SMILES: O=C(O)CCCC[C@@H]1SC[C@@H]2NC(=O)N[C@@H]21]

The chemical structure for streptavidin is reproduced below:

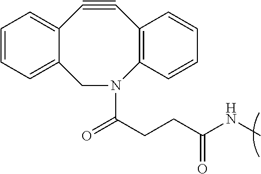 [SMILES: CNC(=O)CCC(=O)N1Cc2ccccc2C#Cc2ccccc21]

Streptavidin contains four biotin binding sites. Even after a streptavidin is functionalized onto the bottom surface of the sample well, three additional binding sites remain for conjugation to a biotin or a biotin-conjugated polymer.

In some embodiments, the bottom surface is functionalized with a silane comprising biotin, or an analog thereof. In some embodiments, the bottom surface is functionalized with a silane comprising a poly(ethylene) glycol chain, wherein the poly(ethylene glycol) chain comprises biotin. In certain embodiments, the bottom surface is functionalized with a mixture of silanes, wherein at least one type of silane comprises biotin and at least one type of silane does not comprise biotin. In some embodiments, the mixture comprises about 10-fold less, about 25-fold less, about 50-fold less, about 100-fold less, about 250-fold less, about 500-fold less, or about 1000-fold less of the biotinylated silane than the silane not comprising biotin. In some aspects, the disclosure provides methods and compositions for modifying a surface.

In some embodiments, a functionalizing agent (e.g., a coupling moiety) and silane can be provided in a ratio that is determined based on a desired density of coupling moiety on the surface to be functionalized. For example, in some embodiments, a functionalized surface is formed using a mixture comprising a functionalizing agent and a silane in a molar ratio of at least 5-fold excess silane over functionalizing agent. In some embodiments, the mixture comprises between about 5-fold excess and about 250-fold excess silane over functionalizing agent (e.g., between about 5-fold and about 100-fold, between about 5-fold and about 50-fold, between about 50-fold and about 250-fold, between about 100-fold and about 250-fold, or between about 50-fold and about 150-fold excess silane over functionalizing agent).

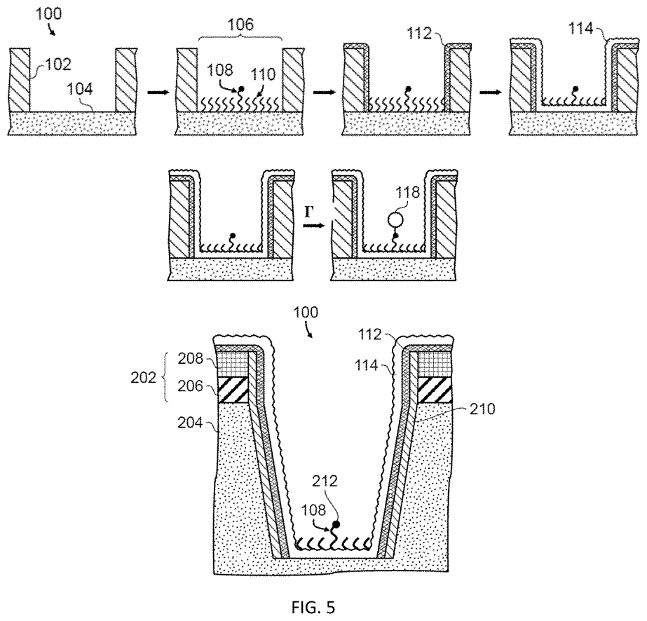

FIG. 5 schematically illustrates an example of sample well surface functionalization in accordance with aspects of the disclosure. A cross-sectional view of a sample well 100 is shown, the sample well having a metallic surface 102 and a silica surface 104. For illustrative purposes, sample well 100 is depicted as being defined by side walls extending from a top surface to a bottom surface, where metallic surface 102 is formed on the side walls and top surface, and silica surface 104 is formed on the bottom surface.

It should be appreciated that, in some embodiments, any of the features defining sample well 100 (side walls, top surface, bottom surface) may have different or additional surface properties. For example, in some embodiments, sample well 100 is defined by side walls extending into the material of the bottom surface, such that one portion of silica surface 104 is formed on the side walls and another portion of silica surface 104 is formed on the bottom surface. In this configuration, the side walls of sample well 100 would include silica surface 104 formed on a surface portion proximal to the bottom surface and metallic surface 102 formed on a surface portion distal to the bottom surface.

In some embodiments, sample well 100 is treated with a functionalizing agent 108 that preferentially binds silica surface 104 to form a functionalized surface 106. The functionalizing agent 108 comprises a coupling moiety which provides a coupling functionality to the bottom surface of sample well 100. As shown, in some embodiments, functionalized surface 106 comprises functionalizing agent 108 and a silane 110 that does not comprise the coupling moiety. Accordingly, in some embodiments, functionalized surface 106 is formed in certain disclosed processes by treating sample well 100 with a mixture comprising functionalizing agent 108 and silane 110. In some embodiments, functionalizing agent 108 is a biotinylated silane (e.g., biotin-PEG-silane) and silane 110 is a non-biotinylated silane (e.g., PEG-silane). In some embodiments, the functionalizing agent binds a PEG-PLL copolymer (e.g., biotin-PEG-PLL).

In some embodiments, a functionalizing agent as described herein comprises a coupling moiety. In some embodiments, the coupling moiety is a covalent coupling moiety. Examples of covalent coupling moieties include, without limitation, a trans-cyclooctene (TCO) moiety, a tetrazine moiety, an azide moiety, an alkyne moiety, an aldehyde moiety, a methyl ether moiety, an isocyanate moiety, an N-hydroxysuccinimide moiety, a thiol moiety, an alkene moiety, a dibenzocyclooctyl moiety, a bicyclononyne moiety, and a thiamine pyrophosphate moiety. Examples of functionalizing agents that comprise a covalent coupling moiety include, without limitation, azide-silanes and azide-organosilanes, such as azide-PEG-silane (e.g., azide-PEG₃-silane, azide-PEG₅-silane) and azide-alkylsilane (e.g., azide-C₁₁-silane). In some embodiments, the coupling moiety is a non-covalent coupling moiety. Examples of coupling moieties include, without limitation, a biotin moiety, an avidin protein, a streptavidin protein, a lectin protein, a SNAP-tag, a methyl ether, and a biotin-streptavidin complex.

In some embodiments, the coupling moiety comprises an avidin protein. Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof. In some embodiments, the avidin protein is streptavidin. The multivalency of avidin protein can allow for various linkage configurations, as each of the four binding sites are independently capable of binding a biotin molecule.

In some embodiments, a non-covalent linkage is formed by an avidin protein bound to a first bis-biotin moiety and a second bis-biotin moiety.

In some embodiments, the coupling moiety functionalized to the sample well surface is contacted with one or more copolymers. In some embodiments, the one or more copolymers comprise a grafted copolymer. In some embodiments, the copolymer is a block copolymer. In some embodiments, the copolymer is a branched copolymer. In some embodiments, the sample well is contacted with the copolymer prior to being contacted with a functionalizing agent. In some embodiments, the sample well is contacted with the functionalizing agent prior to being contacted with the copolymer.

In certain single molecule sequencing methods, a molecule to be analyzed is immobilized onto surfaces such that the molecule may be monitored without interference from other reaction components in solution. In some embodiments, surface immobilization of the molecule allows the molecule to be confined to a desired region of a surface for real-time monitoring of a reaction involving the molecule.

Accordingly, in some aspects, the disclosure provides methods of immobilizing a peptide to a surface by attaching any one of the compounds described herein to a surface of a solid support. In some embodiments, the methods comprise contacting a coupling moiety to a surface of a solid support. In some embodiments, the surface is functionalized with a complementary functional moiety configured for attachment (e.g., covalent or non-covalent attachment) to a functionalized terminal end of a peptide. In some embodiments, the solid support comprises a plurality of sample wells formed at the surface of the solid support. In some embodiments, the methods comprise immobilizing a single peptide to a surface of each of a plurality of sample wells. In some embodiments, confining a single peptide per sample well is advantageous for single molecule detection methods, e.g., single molecule peptide sequencing. In some embodiments, the methods comprise immobilizing a biomolecule other than a peptide to a surface of each of a plurality of sample wells. In some embodiments, the methods comprise immobilizing a single DNA polymerase and/or a nucleic acid to a surface of each of a plurality of sample wells using a coupling moiety (see FIG. 6B).

Provided herein are methods of identifying a single molecule in a sample containing a biomolecule that comprise i) a method of surface functionalization, and ii) a method of single molecule sequencing. In an exemplary method of sample well surface functionalization of the disclosure, the bottom surface of a sample well is first functionalized (or loaded) with a coupling moiety (e.g., a biotin moiety or an azide moiety), so that the moiety is immobilized onto the sample well surface. Subsequently, the coupling moiety is coupled with a copolymer (e.g., PEG-PLL) comprising a triplet state quenching moiety (e.g., trolox, TQ, NBA, or COT). In some embodiments, the copolymer has been grafted in a high-density synthesis with one of trolox, TQ, NBA or COT. After being coupled with a copolymer, the resulting conjugate or complex is herein referred to as a “coupling moiety-copolymer conjugate”. Exemplary coupling moiety-copolymer conjugates of the disclosure include, but are not limited to, Biotin-PEG-PLL grafted with trolox, Biotin-PEG-PLL grafted with TQ, Biotin-PEG-PLL grafted with NBA, Biotin-PEG-PLL grafted with COT, azide (N₃)-PEG-PLL grafted with trolox, azide (N₃)-PEG-PLL grafted with TQ, azide-PEG-PLL grafted with NBA, and azide-PEG-PLL grafted with COT (see FIG. 6A). In this manner, the quenching moiety remains substantially or completely immobilized in a target volume.

In exemplary embodiments of the disclosed methods, the step of contacting the sample well with the sample results in the coupling of a biomolecule with the immobilized coupling moiety (e.g., a biotin). In some embodiments, a DNA polymerase biomolecule is coupled to the biotin moiety (see FIG. 6B).

In some embodiments, the step of contacting the sample well with the sample results in the coupling of the biomolecule (e.g., a DNA complex or a polypeptide) with the biotin moiety. In some embodiments, the step of contacting results in the coupling of the biomolecule with a methyl ether moiety. In some embodiments, the copolymer comprises a Biotin-PEG-PLL. (See FIG. 8 (annotated by “F”).)

In some embodiments, the methods further comprise functionalizing the bottom surface of the sample well with a second coupling moiety. In some embodiments, the copolymer is coupled with the first coupling moiety, and the step of contacting results in the biomolecule being coupled with the second coupling moiety. In other embodiments, the biomolecule is coupled or conjugated to the first coupling moiety, and the second coupling moiety remains unconjugated. In some embodiments, the biomolecule is conjugated to the first coupling moiety, and the copolymer is conjugated to the second coupling moiety. The second coupling moiety may comprise a biotin moiety, an avidin protein, a streptavidin protein, an azide moiety, an alkyne moiety, a ketone moiety, a hydroxylamine moiety. The second coupling moiety may comprise a click chemistry handle. In particular embodiments, the second coupling moiety is an azide moiety. In particular embodiments, the biomolecule is conjugated to a first coupling moiety comprising a biotin moiety, and the copolymer is conjugated to the second coupling moiety comprising an azide moiety. (See FIG. 9, annotated by “E” and “F,” respectively

In some embodiments, the copolymer comprises an azide-PEG-PLL.

In additional exemplary embodiments of the disclosed methods of surface functionalization, the methods further comprise functionalizing (or loading) a streptavidin protein to form a biotin-streptavidin complex at the bottom surface of the well. (FIG. 8 (a first functionalized biotin molecule is indicated as a “biotin terminal”).) In a subsequent step, a copolymer comprising a quenching moiety is added to the sample well. In some embodiments, this copolymer comprises a biotinylated PEG-PLL copolymer comprising a quenching moiety, which is capable of binding the functionalized biotin-streptavidin complex. These embodiments take advantage of the multiple biotin binding sites in a streptavidin protein.

As used herein, in some embodiments, a “surface” refers to a surface of a substrate or solid support containing one or more sample wells. In some embodiments, a solid support refers to a material, layer, or other structure having a surface, such as a receiving surface, that is capable of supporting a deposited material, such as a functionalized peptide or DNA polymerase as described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells.

### Click Chemistry Copolymers

In some aspects, the present disclosure provides a method of selective functionalization of a biomolecule (e.g., a peptide or nucleic acid), comprising reacting the coupling moiety with a copolymer comprising a click chemistry handle. In certain embodiments, the copolymer comprising the click chemistry handle comprises a derivative of a hydroxylamine, a ketone, an azide, a tetrazine, a nitrile oxide, an alkyne or a strained alkene.

The term “click chemistry handle,” as used herein, refers to a reactant, or a reactive group, that can partake in a click chemistry reaction (e.g., an electrocyclic reaction to form a 5-membered heterocyclic ring) with a substrate. For example, a strained alkyne, e.g., a cyclooctyne, can be a click chemistry handle since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide can be a partner click chemistry handle to a cyclooctyne or any other alkyne. Exemplary click chemistry handles suitable for use according to some aspects of the disclosure are described herein, for example, in Tables 1 and 2. Other suitable click chemistry handles are known to those of skill in the art.

TABLE 1
Exemplary click chemistry handles and reactions.

| | |
|---|---|
| 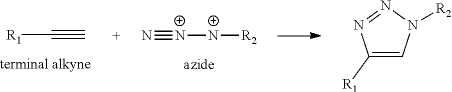 | 1,3-dipolar cycloaddition |
| 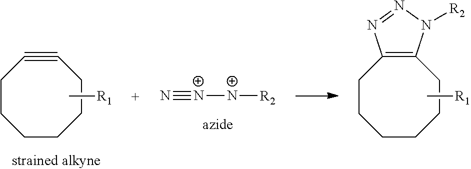 | Strain-promoted cycloaddition |
| 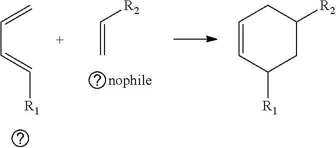 | Diele-Alder reaction |
| 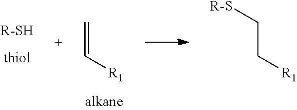 | Thiol-ene reaction |

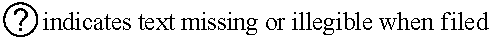

Previously, in order to achieve optimal coupling of a quenching moiety to the surface of a sample well, one of skill in the art needed to test several possible reagents to identify the most suitable copolymers. This testing represented a bottleneck in achievement of rapid single-molecule setups for peptide or nucleotide sequencing. Because most of the quenching moieties of the disclosed methods are hydrophobic, a suitable (or preferred) hydrophilic copolymer may be hydrophilic. The inventors surprisingly found that certain copolymers achieved high coupling efficiencies associated with grafting these moieties onto certain quenching moieties (e.g., trolox, TQ, COT and NBA). In particular, generation of copolymer-quenching moiety conjugates by grafting trolox onto a click chemistry handle such as an azide or PEG-PLL copolymer generated very high coupling efficiencies.

In some embodiments, the copolymer comprises a click chemistry handle that comprises an azide moiety, an alkyne moiety, a ketone moiety, and/or a hydroxylamine moiety. In particular embodiments, the coupling moiety comprises an alkyne moiety. In some embodiments, the coupling moiety comprises a hydroxylamine moiety. In some embodiments, the coupling moiety comprises an azide moiety.

In certain embodiments, the click chemistry handle is a primary alkyne. In certain embodiments, the click chemistry handle is an alkyne that is a cyclic (e.g., mono- or polycyclic) alkyne (e.g., diarylcyclooctyne, or bicycle[6.1.0]nonyne). In certain embodiments, the click chemistry handle is a strained alkene, such as a trans-cyclooctene. In certain embodiments, the click chemistry handle is a moiety comprising an azide. Other exemplary click chemistry handles may comprise an azide, a nitrile oxide, a tetrazine, an alkyne, or a strained alkene. In particular embodiments, the handle comprises bicyclonone (BCN). In other embodiments, the handle comprises dibenzocyclooctyne (DBCO). In certain embodiments, the click chemistry handle comprises a tetrazine, such as a tetrazine comprising the structure:

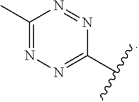 [SMILES: *c1nnc(C)nn1]

In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Such click chemistry handles are well known to those of skill in the art and include the click chemistry handles described in Becer, Hoogenboom, and Schubert, Click Chemistry beyond Metal-Catalyzed Cycloaddition, Angewandte Chemie International Edition (2009) 48: 4900-4908.

TABLE 2
Exemplary click chemistry handles and reactions.

| | Reagent A | Reagent B | Mechanism | Notes on reaction^{[a]} | Reference |
|---|---|---|---|---|---|
| 0 | azide | alkyne | Cu-catalyzed [3 + 2] | 2 h at 60° C. in H₂O | [9] |
| | | | azide-alkyne cycloaddition | | |
| | | | (CuAAC) | | |
| 1 | azide | cyclooctyne | strain-promoted [3 + 2] azide-alkyne cycloaddition | 1 h at RT | [6-8, 10, 11] |
| | | | (SPAAC) | | |
| 2 | azide | activated | [3 + 2] Huisgen cycloaddition | 4 h at 50° C. | [12] |
| | | alkyne | | | |
| 3 | azide | electron-deficient | [3 + 2] cycloaddittion | 12 h at RT in H₂O | [13] |
| | | alkyne | | | |
| 4 | azide | aryne | [3 + 2] cycloaddition | 4 h at RT in THF with crown ether or | [14, 15] |
| | | | | 24 h at RT in CH₃CN | |
| 5 | tetrazine | alkene | Diels-Alder retro-[4 + 2] cycloaddition | 40 min at 25° C. (100% yield) | [36-38] |
| | | | | N₂ is the only by-product | |
| 6 | tetrazole | alkene | 1,3-dipolar cycloaddition | few min UV irradiation and then overnight | [39, 40] |
| | | | (photoclick) | at 4° C. | |
| 7 | dithioester | diene | hetero-Diels-Alder cycloaddition | 10 min at RT | [43] |
| 8 | anthracene | maleimide | [4 + 2] Diels-Alder reaction | 2 days at reflux in toluene | [41] |
| 9 | thiol | alkene | radical addition | 30 min UV (quantitative conv.) or | [19-23] |
| | | | (thio click) | 24 h UV irradiation (>96%) | |
| 10 | thiol | enone | Michael addition | 24 hr at RT in CH₃CN | [27] |
| 11 | thiol | maleimide | Michael addition | 1 h at 40° C. in THF or | [24-26] |
| | | | | 16 h at RT in dioxane | |
| 12 | thiol | para-fluoro | nucleophilic substitution | overnight at RT in DMF or | [32] |
| | | | | 60 min at 40° C. in DMF | |
| 13 | amine | para-fluoro | nucleophilic substitution | 20 min MW at 95° C. in NMP as solvent | [30] |

^{[a]}RT = room temperature, DMF = N,N-dimethylformamide, NMP = N-methylpyrolidone, THF = tetrahydrofuran, CH₃CN = acetonitrile.

### From Becer, Hoogenboom, and Schubert, Click Chemistry Beyond Metal-Catalyzed Cycloaddition, Angewandte Chemie International Edition (2009) 48: 4900-4908.

Additional click chemistry handles suitable for use in methods of surface functionalization described herein are well known to those of skill in the art. Such click chemistry handles include, but are not limited to, the click chemistry reaction partners, groups, and handles described in PCT/US2012/044584.

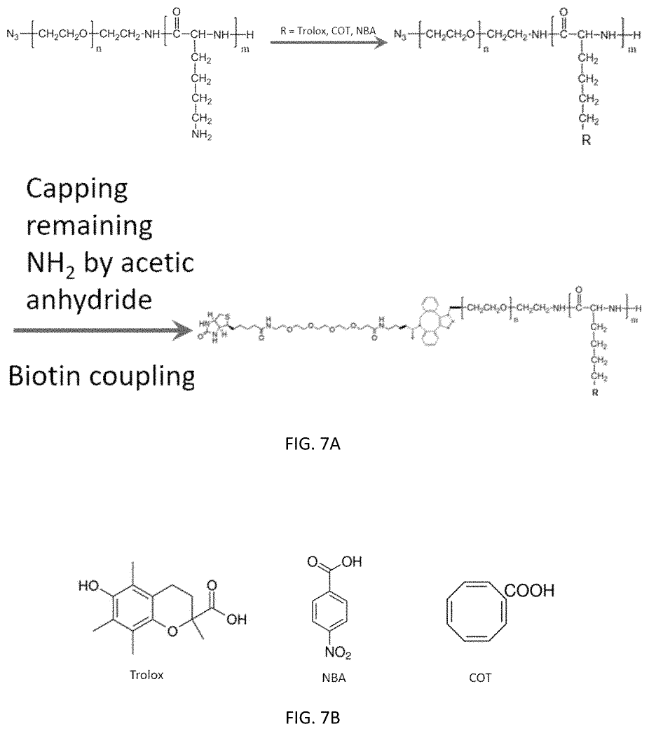

Exemplary copolymers may be generated according to the chemical synthesis shown in FIG. 7A. The disclosure contemplates the use of any grafted PEG-PLL copolymer as the substrate of the synthesis reaction. Such copolymers may contain any number average molecular weight of PEG, and any number average molecular weight of PLL. In some embodiments, number average molecular weights for the components of the copolymers of the disclosure is between 1,000 and 10,000, e.g., between 2,000 and 8,000, between 3,000 and 6,000, or between 3,000 and 5,000. In some embodiments, number average molecular weights for the components of the copolymers of the disclosure is about 1000, 2000, 2500, 3000, 2500, 4000, 4500, 5000, 5500, 6000, 7000, 8000, 9000, or 10,000. Exemplary number average molecular weights for the components of the copolymers of the disclosure may be 5,000 for PEG and 3,000 for PLL.

Any number of branches of PEG and/or PLL are contemplated. In this synthesis reaction, a PEG-PLL copolymer is further grafted onto a quenching moiety selected from trolox, TQ, COT, and NBA. Then, the terminal azide of the PEG-PLL copolymer is converted into an —NH₂ group (amine) using acetic anhydride (“capped”). The resulting amine conjugate is coupled to a biotin molecule using click chemistry techniques. The resulting conjugate may be alternately coupled to an alkyne, hydroxylamine, ketone-hydroxylamine, or azide moiety using click chemistry techniques and reagents.

### Quenching Moieties

In some embodiments, the copolymers described herein comprise one or more quenching moieties (or, as sometimes referred to herein, “anti-bleaching reagents” or “quenching moieties”), such as one or more triplet-state quenching moieties, to absorb or otherwise mitigate photo-induced damage to a labeled molecule and/or other system components (e.g., an enzyme, such as a polymerase or peptidase). Quenching moieties may be used in connection with nucleic acid sequencing and polypeptide sequencing methods in accordance with the disclosure. Quenching moieties may be used with copolymers used for immobilization of nucleic acid and peptide molecules in the methods of the disclosure.

In some embodiments, the quenching moiety is selected from triplet-state quenchers trolox, trolox quinone (TQ), 4-nitrobenzyl alcohol (NBA), and cyclooctatetraene (COT). The chemical structures of trolox, NBA, and COT are shown in FIG. 7B. In certain embodiments, the quenching moiety is trolox. In some embodiments, the at least one quenching moiety comprises a monosaccharide-TEG, a disaccharide, an N-acetyl monosaccharide, a TEMPO-TEG, a trolox-TEG, or a glycerol dendrimer.

In some embodiments, the quenching moiety is grafted onto a copolymer that remains substantially immobilized in a target volume or the nanoaperture of the sample well (e.g., in an evanescent field of the optical waveguide in the target volume). Accordingly, quenching moieties of the disclosure may be substantially immobilized in a target volume during the sequencing methods of the disclosure.

In some embodiments, the quenching moiety refers to any chemical moiety that protects the integrity of a sequencing reaction (e.g., from the damaging effects on the labeled biomolecule by a reactive species generated by an excited state of the label). For example, in some embodiments, the quenching moiety protects the integrity of a sequencing reaction by any of the following: absorbing, quenching, or otherwise mitigating the effects of a reactive species (e.g., reactive oxygen species, free radicals, or any triplet state molecules); increasing read length in a sequencing reaction when compared to a reaction conducted in the absence of the quenching moiety; increasing read accuracy in a sequencing reaction when compared to a reaction conducted in the absence of the quenching moiety; or any combination thereof.

Quenching moieties can comprise any dye molecule that can detectably quench an emission from the one or more luminescent labels described herein. A suitable quenching moiety can be selected based on the specific types of dye molecule(s) of the one or more luminescent labels. For example, in some embodiments, an appropriate quenching moiety possesses an absorption band that exhibits at least some spectral overlap with an emission band of the luminescent label. In some embodiments, this overlap may occur with emission of the donor occurring at a lower or even higher wavelength emission maximum than the maximal absorbance wavelength of the quenching moiety, provided that sufficient spectral overlap exists. In some embodiments, energy transfer may also occur through transfer of emission of the donor to higher electronic states of the acceptor. In some embodiments, an appropriate quenching moiety is capable of absorbing emissions of a particular energy level from the one or more luminescent labels that may be damaging to a polymerase. In such embodiments, a quenching moiety can be selected to preferentially (e.g., selectively) absorb a portion of the emission spectrum that includes emission energy levels potentially damaging to the polymerase without absorbing the portion of the emission spectrum that is being detected as a signal (e.g., during a sequencing reaction).

In exemplary embodiments, the quenching moiety is selected from trolox, trolox quinone (TQ), COT, and NBA. In some embodiments, the quenching moiety is selected from a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine, a carbostyryl, a porphyrin, a salicylate, an anthranilate, an azulene, a perylene, a pyridine, a quinoline, a coumarin (including hydroxycoumarins and aminocoumarins and fluorinated and sulfonated derivatives thereof (as described in U.S. Pat. No. 5,830,912 to Gee et al. (1998) and U.S. Pat. No. 5,696,157 to Wang et al. (1997), each of which is herein incorporated herein by reference), a polyazaindacene (e.g., U.S. Pat. No. 4,774,339 to Haugland, et al. (1988); U.S. Pat. No. 5,187,288 to Kang, et al. (1993); U.S. Pat. No. 5,248,782 to Haugland, et al. (1993); U.S. Pat. No. 5,274,113 to Kang, et al. (1993); U.S. Pat. No. 5,433,896 to Kang, et al. (1995); U.S. Pat. No. 6,005,113 to Wu et al. (1999), all incorporated herein by reference), a xanthene, an oxazine or a benzoxazine, a carbazine (U.S. Pat. No. 4,810,636 to Corey (1989), incorporated by reference), or a phenalenone or benzphenalenone (U.S. Pat. No. 4,812,409 Babb et al. (1989), incorporated by reference).

In some embodiments, the disclosure provides new methods and compositions for identifying single molecules based on one or more luminescent properties of those molecules. In some embodiments, a molecule (e.g., a luminescently labeled nucleoside polyphosphates or amino acid recognition molecule) is identified based on its luminescent lifetime, absorption spectra, emission spectra, luminescent quantum yield, luminescent intensity, or a combination of two or more thereof. Identifying may mean assigning the exact molecular identity of a molecule, or may mean distinguishing or differentiating the particular molecule from a set of possible molecules. In some embodiments, a plurality of single molecules can be distinguished from each other based on different luminescent lifetimes, absorption spectra, emission spectra, luminescent quantum yields, luminescent intensities, or combinations of two or more thereof. In some embodiments, a single molecule is identified (e.g., distinguished from other molecules) by exposing the molecule to a series of separate light pulses and evaluating the timing or other properties of each photon that is emitted from the molecule. In some embodiments, information for a plurality of photons emitted sequentially from a single molecule is aggregated and evaluated to identify the molecule. In some embodiments, a luminescent lifetime of a molecule is determined from a plurality of photons that are emitted sequentially from the molecule, and the luminescent lifetime can be used to identify the molecule. In some embodiments, a luminescent intensity of a molecule is determined from a plurality of photons that are emitted sequentially from the molecule, and the luminescent intensity can be used to identify the molecule. In some embodiments, a luminescent lifetime and luminescent intensity of a molecule is determined from a plurality of photons that are emitted sequentially from the molecule, and the luminescent lifetime and luminescent intensity can be used to identify the molecule.

Aspects of the present application are useful for detecting and/or identifying one or more biological or chemical molecules. In some embodiments, chemical or biological reactions can be evaluated by determining the presence or absence of one or more reagents or products at one or more time points.

Aspects of the present application interrogate a molecule by exposing the molecule to light and determining one or more properties of one or more photons emitted from the molecule. In certain embodiments, the molecule is interrogated by exposing the molecule to a pulse of light and determining one or more properties of a photon emitted from the molecule. In some embodiments, the molecule is exposed to a plurality of separate light pulse events and one or more properties of separate photons emitted after separate light pulse events are determined. In some embodiments, the molecule does not emit a photon in response to each light pulse. However, a plurality of emitted photons can be evaluated by exposing the molecule to a series of separate light pulses and evaluating separate photons that are emitted after a subset of the light pulse events (e.g., photons emitted after about 10% of pulse events, or photons emitted after about 1% of pulse events).

Aspects of the present application are useful to monitor a chemical or biological reaction by determining the presence or absence of one or more reagents, intermediates, and/or products of the reaction at one or more time points. In some embodiments, the progression of a reaction over time can be analyzed by exposing a reaction sample to a series of separate light pulses and analyzing any emitted photon that is detected after each light pulse.

Accordingly, in some aspects of the disclosure, a reaction sample is exposed to a plurality of separate light pulses and a series of emitted photons are detected and analyzed. In some embodiments, the series of emitted photons provides information about a single molecule that is present and that does not change in the reaction sample over the time of the experiment. However, in some embodiments, the series of emitted photons provides information about a series of different molecules that are present at different times in the reaction sample (e.g., as a reaction or process progresses).

In certain embodiments, the frequency of pulsed excitation energies is selected based on the luminescent properties (e.g., luminescent lifetime) of the luminescently labeled molecule (e.g., a luminescently labeled nucleoside polyphosphate or amino acid recognition molecule) to be detected. In some embodiments, the gap between pulses is longer than the luminescent lifetime of one or more luminescently labeled molecules being excited. In some embodiments, the gap is between about two times and about ten times, between about ten times and about 100 times, or between about 100 times and about 1000 times longer than the luminescent lifetime of one or more luminescently labeled molecules being excited. In some embodiments, the gap is about 10 times longer than the luminescent lifetime of one or more luminescently labeled molecules being excited.

In certain embodiments of the nucleic acid sequencing methods of the disclosure, the frequency of pulsed excitation energies is selected based on the chemical process being monitored. For a sequencing reaction the number of pulses delivered to the target volume while a luminescently labeled nucleotide is being incorporated will in part determine the number of emitted photons detected. In some embodiments, the frequency is selected to allow for a sufficient number of photons to be detected during the incorporation of a luminescently labeled nucleotide, wherein a sufficient number is the number of photons necessary to distinguish the luminescently labeled nucleotide from amongst a plurality of types of luminescently labeled nucleotides. In some embodiments, the luminescently labeled nucleotide is distinguished based on the wavelength of the emitted photons. In some embodiments, the luminescently labeled nucleotide is distinguished based on the luminescent emission lifetime, e.g., the time between pulse excitation and emission detection. In some embodiments, the luminescently labeled nucleotide is distinguished based on the wavelength and the luminescent emission lifetime of the emitted photons. In some embodiments, the luminescently labeled nucleotide is distinguished based on the luminescent intensity of the emission signal (e.g., based on the frequency of emission or the total number of emission events within a time period). In some embodiments, the luminescently labeled nucleotide is distinguished based on the luminescent intensity of the emission signal and the luminescent lifetime. In some embodiments, the luminescently labeled nucleotide is distinguished based on the luminescent intensity and the wavelength. In some embodiments, the luminescently labeled nucleotide is distinguished based on the luminescent intensity, the wavelength, and the luminescent lifetime.

### Pixel Array/Chip Architecture

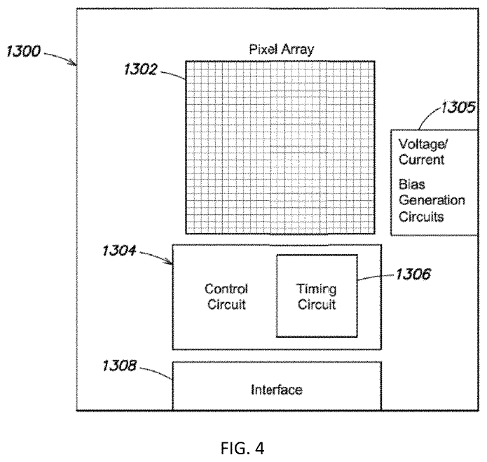

A diagram of an exemplary chip architecture is shown in FIG. 4. An integrated circuit or chip 1300 may include a pixel array 1302 including a plurality of pixels, a control circuit 1304 that includes a timing circuit 1306, voltage/current bias generation circuits 1305 and an interface 1308.

Pixel array 1302 includes an array of pixels 101 laid out in any suitable pattern, such as a rectangular pattern, for example. The pixel array 1302 may have any suitable number of pixels. In some embodiments, the pixel array may have a 64×64 array of 4096 pixels, each including four sub-pixels. However, the techniques described herein are not limited as to the number or arrangement of pixels and sub-pixels included in the pixel array 1302. The pixel array may have row and/or column conductors for reading out rows or columns of the pixel array 1302. Pixels may be read out in parallel, in series, or a combination thereof. For example, in some embodiments a row of pixels may be read out in parallel, and each row of the pixel array may be read out sequentially. However, the techniques described herein are not limited in this respect, as the pixels may be read out in any suitable manner.

The pixel array 1302 is controlled by a control circuit 1304. Control circuit 1304 may be any suitable type of control circuit for controlling operations on the chip 1300, including operations of the pixel array 1302. In some embodiments, control circuit 1304 may include a microprocessor programmed to control operations of the pixel array 1302 and any other operations on the chip 1300. The control circuit may include a computer readable medium (e.g., memory) storing computer readable instructions (e.g., code) for causing the microprocessor to perform such operations. For example, the control circuit 1304 may control producing voltages to be applied to electrodes of the charge carrier segregation structure(s) in each pixel. The control circuit 1304 may change the voltages of one or more electrodes, as discussed above, to capture carriers, transfer carriers, and to perform readout of pixels and the array. The control circuit may set the timing of operations of the charge carrier segregation structure based on a stored timing scheme. The stored timing scheme may be fixed, programmable and/or adaptive, as discussed above.

The control circuit 1304 may include a timing circuit 1306 for timing operations of the charge carrier segregation structure(s) of the pixels or other operations of the chip. In some embodiments, timing circuit 1306 may enable producing signals to precisely control the timing of voltage changes in the charge carrier segregation structure(s) to accurately time bin charge carriers. In some embodiments the timing circuit 1306 may include an external reference clock and/or a delay-locked loop (DLL) for precisely setting the timing of the signals provided to the charge carrier segregation structure(s). In some embodiments, two single-ended delay lines may be used, each with half the number of stages aligned 180-degrees out of phase. However, any suitable technique may be used for controlling the timing of signals on the chip.

The chip 1300 may include an interface 1308 for sending signals from the chip 1300, receiving signals at the chip 1300, or both. The interface 1308 may enable reading out the signals sensed by the pixel array 1302. Readout from the chip 1300 may be performed using an analog interface and/or a digital interface. If readout from the chip 1300 is performed using a digital interface, the chip 1300 may have one or more analog to digital converters for converting signals read out from the pixel array 1302 into digital signals. In some embodiments, the readout circuit may include a Programmable Gain Amplifier. One or more control signals may be provided to the chip 1300 from an external source via interface 1308. For example, such control signals may control the type of measurements to be performed, which may include setting the timing of the time bins.

Analysis of signals read out from the pixel array 1302 may be performed by circuitry on-chip or off-chip. For example, in the context of a luminescent lifetime measurement, analysis of the timing of photon arrival may include approximating a luminescent lifetime of a fluorophore. Any suitable type of analysis may be performed. If analysis of signals read out from the pixel array 1302 is performed on-chip, chip 1300 may have any suitable processing circuitry for performing the analysis. For example, chip 1300 may have a microprocessor for performing analysis that is part of or separate from control circuit 1304. If analysis is performed on-chip, in some embodiments the result of the analysis may be sent to an external device or otherwise provided off-chip through interface 1308. In some embodiments all or a portion of the analysis may be performed off-chip. If analysis is performed off-chip, the signals read out from the pixel array 1302 and/or the result of any analysis performed by the chip 1300, may be provided to an external device through interface 1308.

In some embodiments, the chip 1300 may include one or more of the following:

1) on-chip, digitally controlled, pixel bias generators (DACs).

2) on-chip, digitally programmable gain amplifiers that convert the single-ended pixel output voltage signal to a differential signal and applies gain to the signal

3) digitally-controlled amplifier bias generators that allow scaling the power dissipation with the output rate.

In an embodiment in which the chip 1300B is configured for detection of molecules, the light monitoring sensors may enable alignment of the chip 1300B with a waveguide that Receives Light From One Or More Locations In Which The Molecules Are Positioned.

Molecules that Interact with a Biomolecule

A molecule that interacts with (or, a “molecule interacting with”) a biomolecule, as described herein, is a molecule with binding specificity for, and/or a functional unit that has binding specificity with, a biomolecule of interest in a sample. Generally, a molecule interacting with a biomolecule of the disclosure may comprise a binding moiety having affinity for a biomolecule of interest. In some embodiments, the molecule interacting with a biomolecule is not immobilized to a surface. In some embodiments, the molecule interacting with a biomolecule is immobilized (e.g., functionalized) to a surface of the sample well (e.g., a bottom surface of the sample well). Molecules that interact with a biomolecule may be referred to herein as “affinity reagents.”

In some embodiments, a molecule interacting with a biomolecule comprises a functional unit that specifically interacts with a biomolecule. A functional unit that specifically interacts with a biomolecule may be, in some embodiments, an electrophilic warhead that can react with side chains of individual amino acids (e.g., cysteine or lysine) of a protein biomolecule. In some embodiments, a functional unit that interacts with an biomolecule is any chemical functional group that is capable of reacting with an N-terminus, C-terminus, backbone, or side chain of a protein biomolecule. In some embodiments, the molecule interacting with a biomolecule is an amino acid recognition molecule (or “amino acid recognizer”). In some embodiments, the molecule interacting with a biomolecule is an amino acid recognition molecule that has binding specificity for an internal amino acid of a polypeptide biomolecule. In some embodiments, the molecule interacting with a biomolecule is a terminal amino acid recognition molecule.

In some embodiments, any of the molecules interacting with a biomolecule of interest disclosed herein has a binding affinity of at least 10 pM, 25 pM, 50 pM, 75 pM, 100 pM, 125 pM, 150 pM, 200 pM, 350 pM, 500 pM, 750 pM, or 1000 pM. In some embodiments, the interaction with the biomolecule may comprise a non-covalent interaction.

In various embodiments, the molecule interacting with a biomolecule is conjugated to a label, such as a fluorophore (e.g., a dye). In some embodiments, the molecule interacting with a biomolecule is further conjugated to a second label.

The disclosed methods detect at least one characteristic of the first label and/or the second label. In some embodiments, the step of detection of one or more characteristics of luminescent lifetime, luminescent intensity, luminescent wavelength, retention time, pulse duration, or interpulse duration is dependent on a duration of interaction (e.g., the binding kinetics) of the biomolecule with the molecule interacting with the biomolecule. For instance, the step of detection of one or more characteristics may depend on the duration of time that the biomolecule and molecule interacting with the biomolecule are in the detection region of the sample well.

In some embodiments, the one or more biomolecules are polypeptides or proteins, and the molecule interacting with a biomolecule is a molecule that binds to at least one of the one or more peptide biomolecules. In some embodiments, the molecule interacting with a biomolecule is an amino acid recognition molecule that recognizes an amino acid of the polypeptide, e.g., an internal or terminal amino acid of the polypeptide. In some embodiments, the amino acid recognition molecule is an aptamer or an antibody. In some embodiments, the molecule interacting with a biomolecule binds to the protein biomolecule with a binding affinity of 10⁻¹² to 10⁻¹¹ M, 10⁻¹¹ to 10⁻¹⁰ M, 10⁻¹⁰ to 10⁻⁹ M, 10⁻⁹ to 10⁻⁸ M, 10⁻⁸ 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M. In some embodiments, the binding affinity is in the picomolar to nanomolar range (e.g., between about 10⁻¹² and about 10⁻⁹ M). In some embodiments, the binding affinity is in the nanomolar to micromolar range (e.g., between about 10⁻⁹ and about 10⁻⁶ M). In some embodiments, the binding affinity is in the micromolar to millimolar range (e.g., between about 10⁻⁶ and about 10⁻³ M). In some embodiments, the binding affinity is in the picomolar to micromolar range (e.g., between about 10⁻¹² and about 10⁻⁶ M). In some embodiments, the binding affinity is in the picomolar to millimolar range (e.g., between about 10⁻¹² and about 10⁻³ M).

In some aspects, the application provides amino acid recognition molecules comprising a shielding element, e.g., for enhanced photostability in polypeptide sequencing reactions. In some aspects, the application provides an amino acid recognition molecule of Formula (I):

A-(Y)ₙ-D (I),

wherein: A is an amino acid binding component comprising at least one amino acid recognition molecule; each instance of Y is a polymer that forms a covalent or non-covalent linkage group; n is an integer from 1 to 10, inclusive; and D is a label component comprising at least one detectable label. In some embodiments, D is less than 200 Å in diameter. In some embodiments, —(Y)ₙ— is at least 2 nm in length (e.g., at least 5 nm, at least 10 nm, at least 20 nm, at least 30 nm, at least 50 nm, or more, in length). In some embodiments, —(Y)ₙ— is between about 2 nm and about 200 nm in length (e.g., between about 2 nm and about 100 nm, between about 5 nm and about 50 nm, or between about 10 nm and about 100 nm in length). In some embodiments, each instance of Y is independently a biomolecule or a dendritic polymer (e.g., a polyol, a dendrimer). In some embodiments, the application provides a composition comprising the amino acid recognition molecule of Formula (I). In some embodiments, the amino acid recognition molecule is soluble in the composition. In some aspects, the application provides an amino acid recognition molecule of Formula (II):

A-Y¹-D (II),

wherein: A is an amino acid binding component comprising at least one amino acid recognition molecule; Y¹ is a nucleic acid or a polypeptide; D is a label component comprising at least one detectable label. In some embodiments, when Y¹ is a nucleic acid, the nucleic acid forms a covalent or non-covalent linkage group. In some embodiments, provided that when Y¹ is a polypeptide, the polypeptide forms a non-covalent linkage group characterized by a dissociation constant (K_{D}) of less than 50×10⁻⁹ M. In some embodiments, the K_{D} is less than 1×10⁻⁹ M, less than 1×10⁻¹⁰ M, less than 1×10⁻¹¹ M, or less than 1×10⁻¹² M.

As used herein, in some embodiments, the terms “selective” and “specific” (and variations thereof, e.g., selectively, specifically, selectivity, specificity) refer to a preferential binding interaction. For example, in some embodiments, a molecule interacting with a biomolecule that selectively binds one type of biomolecule preferentially binds the one type over another type of biomolecule. A selective binding interaction will discriminate between one type of biomolecule (e.g., one type of terminal amino acid) and other types of biomolecules (e.g., other types of terminal amino acids), typically more than about 10- to 100-fold or more (e.g., more than about 1,000- or 10,000-fold). As used herein, a terminal amino acid may refer to an amino-terminal amino acid of a polypeptide or a carboxy-terminal amino acid of a polypeptide. Accordingly, it should be appreciated that a selective binding interaction can refer to any binding interaction that is uniquely identifiable to one type of biomolecule over other types of biomolecules. For example, in some aspects, the disclosure provides methods of polypeptide sequencing by obtaining data indicative of association of one or more molecule interacting with a biomolecules with a polypeptide molecule. In some embodiments, the data comprises a series of signal pulses corresponding to a series of reversible molecule binding interactions with an biomolecule of the polypeptide molecule, and the data may be used to determine the identity of the biomolecule. As such, in some embodiments, a “selective” or “specific” binding interaction refers to a detected binding interaction that discriminates between one type of biomolecule and other types of biomolecules.

In some embodiments, a molecule interacting with a biomolecule binds at least one type of biomolecule with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹. The value for k_{off} for any of the disclosed recognition molecules may be a known literature value, or the value can be determined empirically. For example, the value for k_{off} can be measured in a single-molecule assay. In some embodiments, the value for k_{off} can be determined empirically based on signal pulse information obtained in a single-molecule assay. For example, the value for k_{off} can be approximated by the reciprocal of the mean pulse duration. In some embodiments, a molecule interacting with a biomolecule binds two or more types of biomolecules with a different k_{off} for each of the two or more types. In some embodiments, a first k_{off} for a first type of biomolecule differs from a second k_{off} for a second type of biomolecule by at least 10% (e.g., at least 25%, at least 50%, at least 100%, or more). In some embodiments, the first and second values for k_{off} differ by about 10-25%, 25-50%, 50-75%, 75-100%, or more than 100%, for example by about 2-fold, 3-fold, 4-fold, 5-fold, or more.

Amino acid recognition molecules of the disclosure include, for example, proteins and nucleic acids, which may be synthetic or recombinant. In some embodiments, a recognition molecule may be an antibody or an antigen-binding portion of an antibody, an SH2 domain-containing protein or fragment thereof, or an enzymatic biomolecule, such as a peptidase, an aminotransferase, a ribozyme, an aptazyme, an aptamer, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some embodiments, a recognition molecule of the application is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, a recognition molecule is an N-end rule pathway protein selected from a Gid protein (e.g., Gid4 or Gid10 protein), a UBR box protein (e.g., UBR1, UBR2) or UBR box domain-containing protein fragment thereof, a p62 protein or ZZ domain-containing fragment thereof, and a ClpS protein (e.g., ClpS1, ClpS2).

In some embodiments, a recognition molecule of the application is a ClpS protein, such as Agrobacterium tumifaciens ClpS1, Agrobacterium tumifaciens ClpS2, Synechococcus elongatus ClpS1, Synechococcus elongatus ClpS2, Thermosynechococcus elongatus ClpS, Escherichia coli ClpS, or Plasmodium falciparum: ClpS. In some embodiments, the recognition molecule is an L/F transferase, such as Escherichia coli leucyl/phenylalanyl-tRNA-protein transferase. In some embodiments, the recognition molecule is a D/E leucyltransferase, such as Vibrio vulnificus Aspartate/glutamate leucyltransferase Bpt. In some embodiments, the recognition molecule is a UBR protein or UBR-box domain, such as the UBR protein or UBR-box domain of human UBR1 and UBR2 or Saccharomyces cerevisiae UBR1. In some embodiments, the recognition molecule is a p62 protein, such as H. sapiens p62 protein or Rattus norvegicus p62 protein, or truncation variants thereof that minimally include a ZZ domain. In some embodiments, the recognition molecule is a Gid4 protein, such as H. sapiens GID4 or Saccharomyces cerevisiae GID4. In some embodiments, the recognition molecule is a Gid10 protein, such as Saccharomyces cerevisiae GID10. In some embodiments, the recognition molecule is an N-meristoyltransferase, such as Leishmania major N-meristoyltransferase or H. sapiens N-meristoyltransferase NMT1. In some embodiments, the recognition molecule is a BIR2 protein, such as Drosophila melanogaster BIR2. In some embodiments, the recognition molecule is a tyrosine kinase or SH2 domain of a tyrosine kinase, such as H. sapiens Fyn SH2 domain, H. sapiens Src tyrosine kinase SH2 domain, or variants thereof, such as H. sapiens Fyn SH2 domain triple mutant superbinder. In some embodiments, the recognition molecule is an antibody or antibody fragment, such as a single-chain antibody variable fragment (scFv).

In some embodiments, any molecule interacting with a biomolecule described herein may be fixed, or immobilized, to a solid support structure or surface. The solid support structure or surface may be magnetic and/or may be a chip.

In some embodiments, a biomolecule is a nucleic acid, and the molecule interacting with the biomolecule is a labeled nucleotide that is incorporated into a growing nucleic acid strand that is complementary to the biomolecule.

### Nucleic Acid Sequencing

In some embodiments, aspects of the disclosure can be used to assay biological samples, for example to determine the sequence of one or more nucleic acids in the sample and/or to determine the presence or absence of one or more nucleic acid variants (e.g., one or more mutations in a gene of interest) in the sample. In some embodiments, tests can be performed on patient samples (e.g., human patient samples) to provide nucleic acid sequence information or to determine the presence or absence of one or more nucleic acids of interest for diagnostic, prognostic, and/or therapeutic purposes. In some examples, diagnostic tests can include sequencing a nucleic acid molecule in a biological sample of a subject, for example by sequencing cell free deoxyribonucleic acid (DNA) molecules in a biological sample of the subject.

In some embodiments, methods, compositions, and devices described in the disclosure can be used to identify a series of nucleotide monomers that are incorporated into a nucleic acid (e.g., by detecting a time-course of incorporation of a series of labeled nucleotide). In some embodiments, methods, compositions, and devices described in the disclosure can be used to identify a series of nucleotides that are incorporated into a template-dependent nucleic acid sequencing reaction product synthesized by a polymerase enzyme.

The term “nucleic acid,” as used herein, generally refers to a molecule comprising one or more nucleic acid subunits. A nucleic acid may include one or more subunits selected from adenine (A), cytosine (C), guanine (G), thymine (T), and uracil (U), or variants thereof. In some examples, a nucleic acid is deoxyribonucleic acid (DNA) or ribonucleic acid (RNA), or derivatives thereof. A nucleic acid may be single-stranded or double stranded. A nucleic acid may be circular.

The term “nucleotide,” as used herein, generally refers to a nucleic acid subunit, which can include A, C, G, T or U, or variants or analogs thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (e.g., A or G, or variant or analogs thereof) or a pyrimidine (e.g., C, T or U, or variant or analogs thereof). A subunit can enable individual nucleic acid bases or groups of bases (e.g., AA, TA, AT, GC, CG, CT, TC, GT, TG, AC, CA, or uracil-counterparts thereof) to be resolved.

According to some aspects of the disclosure for sequencing DNA molecules, luminescent labels can damage DNA polymerases in a sequencing reaction that is exposed to excitation light. In some aspects, this damage occurs during the incorporation of a luminescently labeled nucleotide, when the luminescent molecule is held in close proximity to the polymerase enzyme. Non-limiting examples of damaging reactions include the formation of a covalent bond between the polymerase and luminescent molecule and emission of radiative or non-radiative decay from the luminescent molecule to the enzyme. This can shorten the effectiveness of the polymerase and reduce the length of a sequencing run. This damage may be minimized or eliminated using the methods disclosed herein.

In some embodiments, nucleic acid sequencing comprises providing a nucleic acid of interest that is immobilized to a surface of a solid support (e.g., attached to a bottom surface of a sample well) through a coupling moiety or a coupling moiety-copolymer conjugate. In some embodiments, the coupling moiety-copolymer conjugate is formed by a non-covalent linkage between a copolymer and a biotin coupling moiety or a streptavidin coupling moiety conjugated to the surface of a well (e.g., functionalized in accordance with aspects of the disclosure) (see, e.g., FIG. 8).

In some aspects, a method of sequencing a nucleic acid biomolecule of interest (or “target nucleic acid”) is provided. In some embodiments, the method of sequencing a target nucleic acid comprises steps of: (i) providing a mixture comprising (a) said target nucleic acid, (b) a primer complementary to said target nucleic acid, (c) a nucleic acid polymerase, and (d) (free) nucleotides for incorporation into a growing nucleic acid strand complementary to said target nucleic acid, wherein said nucleotides include different types of luminescently labeled nucleotides, wherein said luminescently labeled nucleotides yield detectable signals during sequential incorporation into said growing nucleic acid strand, which detectable signals for said different types of luminescently labeled nucleotides are differentiable from one another in a time domain (e.g., by determining timing and/or frequency of the detectable signals); (ii) subjecting said mixture of (i) to a polymerization reaction under conditions that are sufficient to yield said growing nucleic acid strand by extension of said primer; (iii) measuring said detectable signals from said luminescently labeled nucleotides during sequential incorporation into said growing nucleic acid strand; and (iv) determining the timing and/or frequency of said measured detectable signals from said luminescently labeled nucleotides upon sequential incorporation into said growing nucleic acid strand to identify a time sequence of incorporation of said luminescently labeled nucleotides into said growing nucleic acid strand, thereby determining a sequence of said target nucleic acid.

In some aspects, methods of sequencing are provided that comprise steps of: (i) exposing a complex in a target volume in a sample well to one or more labeled nucleotides, the complex comprising a target nucleic acid or a plurality of nucleic acids present in a sample, at least one primer, and a DNA polymerase; (ii) directing a series of pulses of excitation light towards the target volume; (iii) detecting a plurality of emitted photons from the one or more labeled nucleotides during sequential incorporation into a nucleic acid comprising the primer; and (iv) identifying the sequence of incorporated nucleotides by determining one or more characteristics of the emitted photons. These characteristics may be selected from luminescent lifetime, retention time, luminescent intensity, luminescent wavelength, pulse duration, and/or interpulse duration. In some embodiments, the characteristic is luminescent lifetime.

In some embodiments, the free nucleotides are conjugated to the same type of luminescent label, such that detectable signals for the luminescently labeled nucleotides are differentiable from one another in a time domain (e.g., by determining timing, intensity and/or frequency of the detectable signals). In exemplary embodiments, free nucleotides are conjugated to the same type of label, and the differences in intensities of the detectable signal is measured within a time domain. Nucleotides incorporated into a growing nucleic acid strand complementary to the target nucleic acid by the polymerase will remain in the detection region for longer durations than free nucleotides, and as such their intensities will be larger and/or longer than unincorporated nucleotides.

In some embodiments, the free nucleotides (e.g., four types of nucleotides) are each conjugated to a different type of luminescent label, such that detectable signals for the luminescently labeled nucleotides are differentiable from one another in a time domain (e.g., by determining timing, intensity and/or frequency of the detectable signals). Nucleotides incorporated into a growing nucleic acid strand complementary to the target nucleic acid by the polymerase will remain in the detection region for longer durations than free nucleotides, and as such their intensities will be larger and/or longer than unincorporated nucleotides.

In some embodiments, the target nucleic acid (and/or the nucleic acid polymerase) is immobilized to the surface of the solid support. In some embodiments, the time sequence of incorporation is identified subsequent to subjecting the mixture of (i) to the polymerization reaction.

The methods described herein may allow for a set of related nucleic acids (e.g., two or more nucleic acids present in a sample), such as an entire chromosome or genome to be sequenced. In some embodiments, a primer is a sequencing primer. In some embodiments, a sequencing primer can be hybridized to a nucleic acid (e.g., a target nucleic acid) that may or may not be immobilized to a solid support. A solid support can comprise, for example, a sample well on a chip used for nucleic acid sequencing. In some embodiments, a sequencing primer may be immobilized to a solid support and hybridization of the nucleic acid (e.g., the target nucleic acid) further immobilizes the nucleic acid molecule to the solid support. In some embodiments, a polymerase (e.g., DNA polymerase) is immobilized to a solid support and soluble sequencing primer and nucleic acid are contacted to the polymerase. In some embodiments a complex comprising a polymerase, a target nucleic acid and a primer is formed in solution and the complex is immobilized to a solid support (e.g., via immobilization of the polymerase, primer, and/or target nucleic acid). In some embodiments, none of the components are immobilized to a solid support. For example, in some embodiments, a complex comprising a polymerase, a target nucleic acid, and a sequencing primer is formed in situ and the complex is not immobilized to a solid support. In some embodiments, sequencing by synthesis methods can include the presence of a population of target nucleic acid molecules (e.g., copies of a target nucleic acid) and/or a step of amplification (e.g., polymerase chain reaction (PCR)) of a target nucleic acid to achieve a population of target nucleic acids. However, in some embodiments, sequencing by synthesis is used to determine the sequence of a single nucleic acid molecule in any one reaction that is being evaluated and nucleic acid amplification may not be required to prepare the target nucleic acid. In some embodiments, a plurality of single molecule sequencing reactions are performed in parallel (e.g., on a single chip) according to aspects of the instant disclosure. For example, in some embodiments, a plurality of single molecule sequencing reactions are each performed in separate sample wells on a single chip or cartridge.

In some embodiments, one or more molecules that are being analyzed (e.g., interrogated and/or identified) using luminescent lifetime and/or intensity can be labeled molecules (e.g., molecules that have been labeled with one or more luminescent markers). In some embodiments, individual subunits of biomolecules may be identified using markers. In some examples, luminescent markers are used to identify individual subunits of biomolecules. Some embodiments use luminescent markers, which may be exogenous or endogenous markers. Exogenous markers may be external luminescent markers used as a reporter and/or label for luminescent labeling. Examples of exogenous markers may include, but are not limited to, fluorescent molecules, fluorophores, fluorescent dyes, fluorescent stains, organic dyes, fluorescent proteins, species that participate in fluorescence resonance energy transfer (FRET), enzymes, and/or quantum dots. Other exogenous markers are known in the art. Such exogenous markers may be conjugated to a probe or functional group (e.g., molecule, ion, and/or ligand) that specifically binds to a particular target or component. Attaching an exogenous label or reporter to a probe allows identification of the target through detection of the presence of the exogenous label or reporter. Examples of probes may include proteins, nucleic acid (e.g., DNA, RNA) molecules, lipids and antibody probes. The combination of an exogenous marker and a functional group may form any suitable probes, tags, and/or labels used for detection, including molecular probes, labeled probes, hybridization probes, antibody probes, protein probes (e.g., biotin-binding probes), enzyme labels, fluorescent probes, fluorophores, and/or enzyme reporters.

In some embodiments, the luminescent labels among a set of four nucleotides can be selected from dyes comprising an aromatic or heteroaromatic compound and can be a pyrene, anthracene, naphthalene, acridine, stilbene, indole, benzindole, oxazole, carbazole, thiazole, benzothiazole, phenanthridine, phenoxazine, porphyrin, quinoline, ethidium, benzamide, cyanine, carbocyanine, salicylate, anthranilate, coumarin, fluoroscein, rhodamine, or other like compound. Exemplary dyes include xanthene dyes, such as fluorescein or rhodamine dyes, naphthalene dyes, coumarin dyes, acridine dyes, cyanine dyes, benzoxazole dyes, stilbene dyes, pyrene dyes, phthalocyanine dyes, phycobiliprotein dyes, squaraine dyes, BODIPY dyes, and the like.

In some embodiments, the luminescent labels among a set of four nucleotides comprise Alexa Fluor® 546, Cy®3B, Alexa Fluor® 555 and Alexa Fluor® 555, and the FRET pair Alexa Fluor® 555 and Cy®3.5. In some embodiments, the luminescent labels among a set of four nucleotides comprise Alexa Fluor® 555, Cy®3.5, Alexa Fluor® 546, and DyLight® 554-R1. In some embodiments, the luminescent labels among a set of four nucleotides comprise Alexa Fluor® 555, Cy®3.5, ATTO Rho6G, and DyLight® 554-R1. In some embodiments, the luminescent labels among a set of four nucleotides comprise Alexa Fluor® 555, Cy®3B, ATTO Rho6G, and DyLight® 554-R1. In some embodiments, the luminescent labels among a set of four nucleotides comprise Alexa Fluor® 555, Cy®3B, ATTO 542, and DyLight® 554-R1. In some embodiments, the luminescent labels among a set of four nucleotides comprise Alexa Fluor® 555, Cy®3B, ATTO 542, and Alexa Fluor® 546. In some embodiments, the luminescent labels among a set of four nucleotides comprise Cy®3.5, Cy®3B, ATTO Rho6G, and DyLight® 554-R1.

In certain embodiments, at least one type, at least two types, at least three types, or at least four of the types of luminescently labeled nucleotides comprise a luminescent dye selected from the group consisting of 6-TAMRA, 5/6-Carboxyrhodamine 6G, Alexa Fluor® 546, Alexa Fluor® 555, Alexa Fluor® 568, Alexa Fluor® 610, Alexa Fluor® 647, Aberrior Star 635, ATTO 425, ATTO 465, ATTO 488, ATTO 495, ATTO 514, ATTO 520, ATTO Rho6G, ATTO 542, ATTO 647N, ATTO Rho14, Chromis 630, Chromis 654A, Chromeo™ 642, CF™514, CF™532, CF™543, CF™546, CF™546, CF™555, CF™568, CF™633, CF™640R, CF™660C, CF™660R, CF™680R, Cy®3, Cy®3B, Cy®3.5, Cy®5, Cy®5.5, Dyomics-530, Dyomics-547P1, Dyomics-549P1, Dyomics-550, Dyomics-554, Dyomics-555, Dyomics-556, Dyomics-560, Dyomics-650, Dyomics-680, DyLight® 554-R1, DyLight® 530-R2, DyLight® 594, DyLight® 635-B2, DyLight® 650, DyLight® 655-B4, DyLight® 675-B2, DyLight® 675-B4, DyLight® 680, HiLyte™ Fluor 532, HiLyte™ Fluor 555, HiLyte™ Fluor 594, LightCycler® 640R, Seta™ 555, Seta™ 670, Seta™700, Seta™u 647, and Seta™u 665, or are of formulae (Dye 101), (Dye 102), (Dye 103), (Dye 104), (Dye 105), or (Dye 106), as described herein.

In some embodiments, at least one type, at least two types, at least three types, or at least four of the types of luminescently labeled nucleotides each comprise a luminescent dye selected from the group consisting of Alexa Fluor® 546, Alexa Fluor® 555, Cy®3B, Cy®3.5, DyLight® 554-R1, Alexa Fluor® 546, Atto Rho6G, ATTO 425, ATTO 465, ATTO 488, ATTO 495, ATTO 514, ATTO 520, ATTO Rho6G, and ATTO 542.

In some embodiments, a first type of luminescently labeled nucleotide comprises Alexa Fluor® 546, a second type of luminescently labeled nucleotide comprises Cy®3B, a third type of luminescently labeled nucleotide comprises two Alexa Fluor® 555, and a fourth type of luminescently labeled nucleotide comprises Alexa Fluor® 555 and Cy®3.5.

In some embodiments, at least one type, at least two types, at least three types, or at least four of the types of luminescently labeled nucleotides comprise a luminescent dye selected from the group consisting of Alexa Fluor® 532, Alexa Fluor® 546, Alexa Fluor® 555, Alexa Fluor® 594, Alexa Fluor® 610, CF™532, CF™543, CF™555, CF™594, Cy®3, DyLight® 530-R2, DyLight® 554-R1, DyLight® 590-R2, DyLight® 594, DyLight® 610-B1, or are of formulae (Dye 101), (Dye 102), (Dye 103), (Dye 104), (Dye 105), or (Dye 106).

In some embodiments, a first and second type of luminescently labeled nucleotide comprise a luminescent dye selected from the group consisting of Alexa Fluor® 532, Alexa Fluor® 546, Alexa Fluor® 555, CF™532, CF™543, CF™555, Cy®3, DyLight® 530-R2, DyLight® 554-R1, and a third and fourth type of luminescently labeled nucleotide comprise a luminescent dye selected from the group consisting of Alexa Fluor® 594, Alexa Fluor® 610, CF™594, DyLight® 590-R2, DyLight® 594, DyLight® 610-B1, or are of formulae (Dye 101), (Dye 102), (Dye 103), (Dye 104), (Dye 105), or (Dye 106).

In certain embodiments, at least one type, at least two types, at least three types, or at least four of the types of luminescently-labeled nucleotide molecules comprise a luminescent protein selected from the group consisting of TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, monomeric Azami Green, TagGFP2, mUKG, mWasabi, Clover, mNeonGreen, EYFP, Citrine, Venus, SYFP2, TagYFP, monomeric Kusabira-Orange, mKOK, mKO2, mOrange, mOrange2, mRaspberry, mCherry, mStrawberry, mTangerine, tdTomato, TagRFP, TagRFP-T, mApple, mRuby, mRuby2, mPlum, HcRed-Tandem, mKate2, mNeptune, NirFP, TagRFP657, IFPl.4, iRFP, mKeima Red, LSS-mKate1, LSS-mKate2, mBeRFP, PA-GFP, PAmCherryl, PATagRFP, Kaede (green), Kaede (red), KikGR1 (green), KikGR1 (red), PS-CFP2, mEos2 (green), mEos2 (red), PSmOrange, or Dronpa.

Although the present disclosure makes reference to luminescent labels, other types of labels may be used with devices, systems and methods provided herein. Such labels may be mass tags, electrostatic tags, electrochemical labels, or any combination thereof.

While exogenous markers may be added to a sample, endogenous markers may be already part of the sample. Endogenous markers may include any luminescent marker present that may luminesce or “autofluoresce” in the presence of excitation energy. Autofluorescence of endogenous fluorophores may provide for label-free and noninvasive labeling without requiring the introduction of exogenous fluorophores. Examples of such endogenous fluorophores may include hemoglobin, oxyhemoglobin, lipids, collagen and elastin crosslinks, reduced nicotinamide adenine dinucleotide (NADH), oxidized flavins (FAD and FMN), lipofuscin, keratin, and/or porphyrins, by way of example and not limitation.

In certain embodiments, the template-dependent nucleic acid sequencing product is carried out by naturally occurring nucleic acid polymerases. In some embodiments, the polymerase is a mutant or modified variant of a naturally occurring polymerase. In some embodiments, the template-dependent nucleic acid sequence product will comprise one or more nucleotide segments complementary to the template nucleic acid strand. In one aspect, the disclosure provides a method of determining the sequence of a template (or target) nucleic acid strand by determining the sequence of its complementary nucleic acid strand.

In exemplary embodiments of the disclosed methods, the molecule that interacts with a biomolecule, e.g., a nucleic acid biomolecule, is one or more labeled nucleotides (that is, labeled nucleoside polyphosphates).

In some embodiments of the disclosed methods, the molecule that interacts with a nucleic acid biomolecule is a nucleic acid polymerase. In some embodiments, the molecule that interacts with a biomolecule is a complex comprising a nucleic acid polymerase. The term “polymerase,” as used herein, generally refers to any enzyme (or polymerizing enzyme) capable of catalyzing a polymerization reaction. Examples of polymerases include, without limitation, a nucleic acid polymerase, a transcriptase or a ligase. A polymerase can be a polymerization enzyme.

Embodiments directed towards single molecule nucleic acid extension (e.g., for nucleic acid sequencing) may use any polymerase that is capable of synthesizing a nucleic acid complementary to a target nucleic acid molecule. In some embodiments, a polymerase may be a DNA polymerase, an RNA polymerase, a reverse transcriptase, and/or a mutant or altered form of one or more thereof.

Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase (p29 (psi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Tth polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some embodiments, the polymerase is a single subunit polymerase. Non-limiting examples of DNA polymerases and their properties are described in detail in, among other places, DNA Replication 2nd edition, Kornberg and Baker, W. H. Freeman, New York, N.Y. (1991).

Upon base pairing between a nucleobase of a target nucleic acid and the complementary dNTP, the polymerase incorporates the dNTP into the newly synthesized nucleic acid strand by forming a phosphodiester bond between the 3′ hydroxyl end of the newly synthesized strand and the alpha phosphate of the dNTP. In examples in which the luminescent label conjugated to the dNTP is a fluorophore, its presence is signaled by excitation and a pulse of emission is detected during or after the step of incorporation. For detection labels that are conjugated to the terminal (gamma) phosphate of the dNTP, incorporation of the dNTP into the newly synthesized strand results in release of the beta and gamma phosphates and the detection label, which is free to diffuse in the sample well, resulting in a decrease in emission detected from the fluorophore.

In some embodiments, the polymerase is a polymerase with high processivity. However, in some embodiments, the polymerase is a polymerase with reduced processivity. Polymerase processivity generally refers to the capability of a polymerase to consecutively incorporate dNTPs into a nucleic acid template without releasing the nucleic acid template. In some embodiments, the polymerase is a polymerase with low 5′-3′ exonuclease activity and/or 3′-5′ exonuclease. In some embodiments, the polymerase is modified (e.g., by amino acid substitution) to have reduced 5′-3′ exonuclease activity and/or 3′-5′ activity relative to a corresponding wild-type polymerase. Further non-limiting examples of DNA polymerases include 9° Nm™ DNA polymerase (New England Biolabs), and a P680G mutant of the Klenow exopolymerase (Tuske et al. (2000) JBC 275(31):23759-23768). In some embodiments, a polymerase having reduced processivity provides increased accuracy for sequencing templates containing one or more stretches of nucleotide repeats (e.g., two or more sequential bases of the same type). In some embodiments, the polymerase is a polymerase that has a higher affinity for a labeled nucleotide than for a non-labeled nucleic acid.

In other aspects, the disclosure provides methods of sequencing target nucleic acids by sequencing a plurality of nucleic acid fragments, wherein the target nucleic acid comprises the fragments. In certain embodiments, the method comprises combining a plurality of fragment sequences to provide a sequence or partial sequence for the parent target nucleic acid. In some embodiments, the step of combining is performed by computer hardware and software. The methods described herein may allow for a set of related target nucleic acids, such as an entire chromosome or genome to be sequenced.

During sequencing, a polymerizing enzyme may couple (e.g., attach) to a priming location of a target nucleic acid molecule. The priming location can be a primer that is complementary to a portion of the target nucleic acid molecule. As an alternative the priming location is a gap or nick that is provided within a double stranded segment of the target nucleic acid molecule. A gap or nick can be from 0 to at least 1, 2, 3, 4, 5, 6, 7, 8, 9, 10, 20, 30, or 40 nucleotides in length. A nick can provide a break in one strand of a double stranded sequence, which can provide a priming location for a polymerizing enzyme, such as, for example, a strand displacing polymerase enzyme.

In some cases, a sequencing primer can be hybridized to a target nucleic acid molecule that may or may not be immobilized to a solid support. A solid support can comprise, for example, a sample well (e.g., a nanoaperture) on a microchip (or chip) used for nucleic acid sequencing. In some embodiments, a sequencing primer may be immobilized to a solid support and hybridization of the target nucleic acid molecule also immobilizes the target nucleic acid molecule to the solid support. In some embodiments, a polymerase is immobilized to a solid support and soluble primer and target nucleic acid are contacted to the polymerase. However, in some embodiments a complex comprising a polymerase, a target nucleic acid and a primer is formed in solution and the complex is immobilized to a solid support (e.g., via immobilization of the polymerase, primer, and/or target nucleic acid).

Under appropriate conditions, a polymerase enzyme that is contacted to an hybridized primer/target nucleic acid can add or incorporate one or more nucleotides onto the primer, and nucleotides can be added to the primer in a 5′ to 3′, template-dependent fashion. Such incorporation of nucleotides onto a primer (e.g., via the action of a polymerase) can generally be referred to as a primer extension reaction. Each nucleotide can be associated with a detectable label that can be detected and identified (e.g., based on the label's luminescent lifetime and/or other characteristics) during the nucleic acid extension reaction and used to determine each nucleotide incorporated into the extended primer and, thus, a sequence of the newly synthesized nucleic acid molecule. Via sequence complementarity of the newly synthesized nucleic acid molecule, the sequence of the target nucleic acid molecule can also be determined. In some cases, hybridization of a sequencing primer to a target nucleic acid molecule and incorporation of nucleotides to the sequencing primer can occur at similar reaction conditions (e.g., the same or similar reaction temperature) or at differing reaction conditions (e.g., different reaction temperatures). In some embodiments, sequencing by synthesis methods can include the presence of a population of target nucleic acid molecules (e.g., copies of a target nucleic acid) and/or a step of amplification of the target nucleic acid to achieve a population of target nucleic acids. However, in some embodiments sequencing by synthesis is used to determine the sequence of a single molecule in each reaction that is being evaluated (and nucleic acid amplification is not required to prepare the target template for sequencing). In some embodiments, a plurality of single molecule sequencing reactions are performed in parallel (e.g., on a single chip) according to aspects of the present application. For example, in some embodiments, a plurality of single molecule sequencing reactions are each performed in separate reaction chambers (e.g., nanoapertures, sample wells) that have been fabricated onto a single chip.

In some embodiments, the disclosed nucleic acid sequencing methods are performed in sample wells that have been fabricated on a complementary metal oxide semiconductor (CMOS) chip. Each well may be aligned over a CMOS photodiode. In some embodiments, the disclosed nucleic acid sequencing methods are performed on a CMOS chip in conjunction with benchtop integrated device. In some embodiments, the sequencing methods are performed on a CMOS chip in conjunction with a one- or multiple-channel photodetection sequencing-by-synthesis integrated device.

In some embodiments, the nucleic acid of the luminescently labeled nucleotide further comprises a third oligonucleotide strand hybridized to at least one of the first and second oligonucleotide strands. In some embodiments, the nucleic acid further comprises a fourth oligonucleotide strand hybridized to at least one of the first, second, and third oligonucleotide strands. In some embodiments, the oligonucleotide strands form a Holliday junction.

In some aspects, the disclosure provides methods of determining the sequence of a template nucleic acid. In some embodiments, the methods include a step comprising exposing a complex in a target volume, the complex comprising the template nucleic acid, a primer, and a polymerizing enzyme, to a plurality of types of luminescently labeled nucleotides. In some embodiments, each type of luminescently labeled nucleotide comprises one or more luminescent labels (e.g., comprise identical luminescent labels, comprise different luminescent labels) connected to one or more nucleoside polyphosphates via a nucleic acid. In some embodiments, the nucleic acid comprises a quenching moiety. Accordingly, in some aspects, the disclosure provides methods of nucleic acid sequencing that utilize any of the luminescently labeled nucleoside polyphosphate compositions described herein.

In some embodiments, the methods further comprise a step of directing a series of pulses of one or more excitation energies towards a vicinity of the target volume. In some embodiments, the methods further comprise a step of detecting a plurality of emitted photons from luminescently labeled nucleotides during sequential incorporation into a nucleic acid comprising the primer. In some embodiments, the methods further comprise a step of identifying the sequence of incorporated nucleotides by determining timing and/or frequency of the emitted photons.

In some embodiments, four different types of nucleotides (e.g., adenine, guanine, cytosine, thymine/uracil) in a reaction mixture can each be labeled with one or more luminescent labels. In some embodiments, each type of nucleotide can be conjugated to more than one of the same luminescent molecule (e.g., two or more of the same fluorescent dye connected to a nucleotide). In some embodiments, each luminescent molecule can be connected to more than one nucleotide (e.g., two or more of the same nucleotide. In some embodiments, all four nucleotides are labeled with luminescent molecules that absorb and emit within the same spectral range (e.g., 520-570 nm).

Embodiments are capable of sequencing single nucleic acid molecules with high accuracy and long read lengths, such as an accuracy of at least about 50%, 60%, 70%, 75%, 80%, 85%, 90%, 92%, 95%, 96%, 97%, 98%, 99%, 99.9%, 99.99%, 99.999%, or 99.9999%, and/or read lengths greater than or equal to about 10 base pairs (bp), 50 bp, 100 bp, 200 bp, 300 bp, 400 bp, 500 bp, 1000 bp, 2,500 bp, 5,000 bp, 6,000 bp, 7,000 bp, 8,000 bp, 9,000 bp, 10,000 bp, 20,000 bp, 30,000 bp, 40,000 bp, 50,000 bp, or 100,000 bp. In exemplary embodiments, the disclosed methods are capable of sequencing single nucleic acid molecules with accuracies of about 85%, about 90%, or about 92%. In some embodiments, the disclosed methods are capable of sequencing single nucleic acid molecules with an accuracy of 91.9%. In exemplary embodiments, the disclosed methods are capable of sequencing single nucleic acid molecules with read lengths of about 8220 base pairs.

In some embodiments, the target nucleic acid molecule used in single molecule sequencing is a single stranded target nucleic acid (e.g., deoxyribonucleic acid (DNA), DNA derivatives, ribonucleic acid (RNA), RNA derivatives) template that is added or immobilized to a sample well (e.g., nanoaperture) containing at least one additional component of a sequencing reaction (e.g., a polymerase such as, a DNA polymerase, a sequencing primer) immobilized or attached to a solid support such as the bottom or side walls of the sample well. The target nucleic acid molecule or the polymerase can be attached to a sample wall, such as at the bottom or side walls of the sample well directly or through a linker. The sample well (e.g., nanoaperture) also can contain any other reagents needed for nucleic acid synthesis via a primer extension reaction, such as, for example suitable buffers, co-factors, enzymes (e.g., a polymerase) and deoxyribonucleoside polyphosphates, such as, e.g., deoxyribonucleoside triphosphates, including deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, that include luminescent labels, such as fluorophores. In some embodiments, each class of dNTPs (e.g., adenine-containing dNTPs (e.g., dATP), cytosine-containing dNTPs (e.g., dCTP), guanine-containing dNTPs (e.g., dGTP), uracil-containing dNTPs (e.g., dUTPs) and thymine-containing dNTPs (e.g., dTTP)) is conjugated to a distinct luminescent label such that detection of light emitted from the tag indicates the identity of the dNTP that was incorporated into the newly synthesized nucleic acid. Emitted light from the luminescent label can be detected and attributed to its appropriate luminescent label (and, thus, associated dNTP) via any suitable device and/or method, including such devices and methods for detection described elsewhere herein. The luminescent label may be conjugated to the dNTP at any position such that the presence of the luminescent label does not inhibit the incorporation of the dNTP into the newly synthesized nucleic acid strand or the activity of the polymerase. In some embodiments, the luminescent label is conjugated to the terminal phosphate (e.g., the gamma phosphate) of the dNTP.

In some embodiments, the single-stranded target nucleic acid template can be contacted with a sequencing primer, dNTPs, polymerase and other reagents necessary for nucleic acid synthesis. In some embodiments, all appropriate dNTPs can be contacted with the single-stranded target nucleic acid template simultaneously (e.g., all dNTPs are simultaneously present) such that incorporation of dNTPs can occur continuously. In other embodiments, the dNTPs can be contacted with the single-stranded target nucleic acid template sequentially, where the single-stranded target nucleic acid template is contacted with each appropriate dNTP separately, with washing steps in between contact of the single-stranded target nucleic acid template with differing dNTPs. Such a cycle of contacting the single-stranded target nucleic acid template with each dNTP separately followed by washing can be repeated for each successive base position of the single-stranded target nucleic acid template to be identified.

In some embodiments, the sequencing primer is hybridized to the single-stranded target nucleic acid template and the polymerase consecutively incorporates the dNTPs (or other deoxyribonucleoside polyphosphate) to the primer based on the single-stranded target nucleic acid template. The unique luminescent label associated with each incorporated dNTP can be excited with the appropriate excitation light during or after incorporation of the dNTP to the primer and its emission can be subsequently detected, using, any suitable device(s) and/or method(s), including devices and methods for detection described elsewhere herein. Detection of a particular emission of light (e.g., having a particular emission lifetime, intensity, spectrum and/or combination thereof) can be attributed to a particular dNTP incorporated. The sequence obtained from the collection of detected luminescent labels can then be used to determine the sequence of the single-stranded target nucleic acid template via sequence complementarity.

While the present disclosure makes reference to dNTPs, devices, systems and methods provided herein may be used with various types of nucleotides, such as ribonucleotides and deoxyribonucleotides (e.g., deoxyribonucleoside polyphosphates with at least 4, 5, 6, 7, 8, 9, or 10 phosphate groups). Such ribonucleotides and deoxyribonucleotides can include various types of tags (or markers). In some embodiments, the present disclosure provides methods and compositions that may be advantageously utilized in the technologies described in U.S. patent application Ser. Nos. 14/543,865, 14/543,867, 14/543,888, 14/821,656, 14/821,686, 14/821,688, 15/161,067, 15/161,088, 15/161,125, 15/255,245, 15/255,303, 15/255,624, 15/261,697, and 15/261,724, the contents of each of which is incorporated herein by reference.

In certain embodiments, the reaction mixture of the nucleic acid sequencing reactions provided herein contains additional reagents. In some embodiments, the reaction mixture comprises a buffer. In some embodiments, a buffer comprises 3-(N-morpholino)propanesulfonic acid (MOPS). In some embodiments, a buffer is present in a concentration of between about 1 mM and between about 100 mM. In some embodiments, the concentration of MOPS is about 50 mM. In some embodiments, the reaction mixture comprises one or more salt. In some embodiments, a salt comprises potassium acetate. In some embodiments, the concentration of potassium acetate is about 140 mM. In some embodiments, a salt is present in a concentration of between about 1 mM and about 200 mM. In some embodiments, the reaction mixture comprises a magnesium salt (e.g., magnesium acetate). In some embodiments, the concentration of magnesium acetate is about 20 mM. In some embodiments, a magnesium salt is present in a concentration of between about 1 mM and about 50 mM. In some embodiments, the reaction mixture comprises a reducing agent. In some embodiments, a reducing agent is dithiothreitol (DTT). In some embodiments, a reducing agent is present in a concentration of between about 1 mM and about 50 mM. In some embodiments, the concentration of DTT is about 5 mM.

In some embodiments, the reaction mixture comprises one or more quenching moieties (or anti-bleaching reagents). In some embodiments, the reaction mixture comprises a triplet state quenching moiety. In some embodiments, a quenching moiety comprises trolox. In some embodiments, a quenching moiety comprises trolox quinone (TQ). In some embodiments, a quenching moiety comprises cyclooctatetraene (COT). In some embodiments, a quenching moiety comprises 4-nitrobenzyl alcohol (NBA). In some embodiments, a quenching moiety is present in a concentration of between about 0.1 mM and about 20 mM. In some embodiments, the concentration of trolox (or TQ) is about 3 mM. In some embodiments, the concentration of NBA is about 3 mM. In some embodiments, the concentration of COT is about 3 mM. A mixture with a quenching moiety (e.g., trolox) may also comprise an enzyme to regenerate the quenching moiety. In some embodiments, the concentration of this enzyme is about 0.3 mM.

During sequencing the method of identifying a nucleotide may vary between various base pairs in the sequence. In certain embodiments, two types of nucleotides may be labeled to absorb at a first excitation energy, and those two types of nucleotides (e.g., A, G) are distinguished based on different luminescent intensity, whereas two additional types of nucleotides (e.g., C, T) may be labeled to absorb at a second excitation energy, and those two additional types of nucleotides are distinguished based on different luminescent lifetime. In some embodiments, between 2 and 4 luminescently labeled nucleotide are be differentiated based on luminescent lifetime. In some embodiments, between 2 and 4 luminescently labeled nucleotides are differentiated based on luminescent intensity. In some embodiments, between 2 and 4 luminescently labeled nucleotides are differentiated based on luminescent lifetime and luminescent intensity.

### Exemplary Nucleic Acid Sequencing Methods

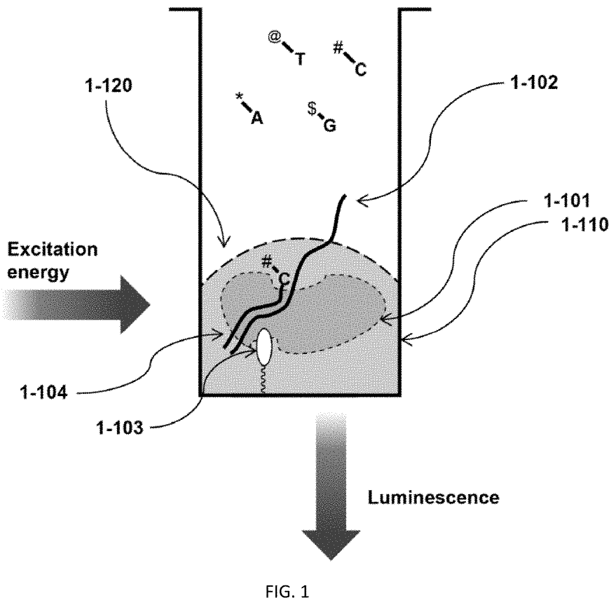

The following example is meant to illustrate some of the methods, compositions and devices described herein. All aspects of the example are non-limiting. FIG. 1 schematically illustrates the setup of a single molecule nucleic acid sequencing method. 1-110 is a sample well (e.g., nanoaperture, reaction chamber) configured to contain a single complex comprising a nucleic acid polymerase 1-101, a target nucleic acid 1-102 to be sequenced, and a primer 1-104. In this example, a bottom region of sample well 1-110 is depicted as a target volume (e.g., the excitation region) 1-120.

The target volume is a volume towards which the excitation energy is directed. In some embodiments, the volume is a property of both the sample well volume and the coupling of excitation energy to the sample well. The target volume may be configured to limit the number of molecules or complexes confined in the target volume. In some embodiments, the target volume is configured to confine a single molecule or complex. In some embodiments, the target volume is configured to confine a single polymerase complex. In FIG. 1 the complex comprising polymerase 1-101 is confined in target volume 1-120. The complex may optionally be immobilized by attachment to a surface of the sample well. Exemplary processes for sample well surface preparation and functionalization are discussed in further detail elsewhere in the disclosure. In this example the polymerase is immobilized by a copolymer 1-103 comprising biotin, or another moiety suitable for attaching the copolymer to the polymerase 1-101.

The volume of the aperture also contains a reaction mixture with suitable solvent, buffers, and other additives necessary for the polymerase complex to synthesize a nucleic acid strand. The reaction mixture also contains a plurality of types of luminescently labeled nucleotides.

FIG. 1 indicates with arrows the concept of an excitation energy being delivered to a vicinity of the target volume, and a luminescence being emitted towards a photodetector. The arrows are schematic and are not meant to indicate the particular orientation of excitation energy delivery or luminescence. In some embodiments, the excitation energy is a pulse of light from a light source. The excitation energy may travel through one or more device components, such as waveguides or filters, between the light source and the vicinity of the target volume. The emission energy may also travel through one or more device components, such as waveguides or filters, between the luminescent molecule and the detector. Some luminescences may emit on a vector which is not directed to the detector (e.g., towards the sidewall of the sample well) and may not be detected.

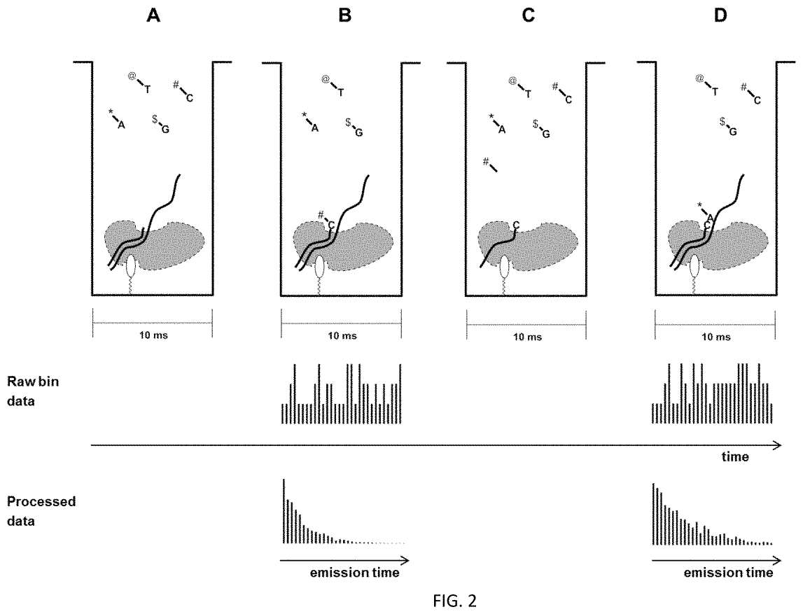

FIG. 2 schematically illustrates a sequencing process in a target volume of a single sample well (e.g., a nanoaperture) over time. Stages A through D depict a sample well with a polymerase complex as in FIG. 1. Stage A depicts the initial state before any nucleotides have been added to the primer. Stage B depicts the incorporation event of a luminescently labeled nucleotide (#-C). Stage C depicts the period between incorporation events. In this example, nucleotide C has been added to the primer, and the label (and linker) previously attached to the luminescently labeled nucleotide (#-C) has been cleaved. Stage D depicts a second incorporation event of a luminescently labeled nucleotide (*-A). The complementary strand after Stage D consists of the primer, a C nucleotide, and an A nucleotide.

Stages A and C both depict the periods before or between incorporation events, which are indicated in this example to last for about 10 milliseconds. In stages A and C, because there is no nucleotide being incorporated, there is no luminescently labeled nucleotide in the target volume (not drawn in FIG. 2), though background luminescence or spurious luminescence from luminescently labeled nucleotide which is not being incorporated may be detected. Stages B and D show incorporation events of different nucleotides (#-C, and *-A, respectively). In this example these events are also indicated to last for about 10 milliseconds.

The row labeled “Raw bin data” depicts the data generated during each Stage. Throughout the example experiment, a plurality of pulses of light are delivered to the vicinity of the target volume. For each pulse a detector is configured to record any emitted photon received by the detector. When an emitted photon is received by the detector it is separated into one of a plurality of time bins, of which there are 3 in this example. In some embodiments, the detector is configured with between 2 and 16 time bins. The “Raw bin data” records a value of 1 (shortest bars), 2 (medium bars), or 3 (longest bars), corresponding to the shortest, middle, and longest bins, respectively. Each bar indicates detection of an emitted photon.

Since there is no luminescently labeled nucleotide present in the target volume for Stage A or C, there are no photons detected. For each of Stage B and D a plurality of photon emission events (luminescent events or “luminescences” as used herein) is detected during the incorporation event. Luminescent label # has a shorter luminescent lifetime than luminescent label *. The Stage B data is thus depicted as having recorded lower average bin values, than Stage D where the bin values are higher.

The row labeled “Processed data” depicts raw data which has been processed to indicate the number (counts) of emitted photons at times relative to each pulse. Since each bar corresponds to the photon count of a particular time bin, the exemplary curves depicting processed data correspond to raw bin data comprising more time bins than the three time bins described in the figure. In this example, the data is only processed to determine luminescent lifetime, but the data may also be evaluated for other luminescent properties, such as luminescent intensity or the wavelength of the absorbed or emitted photons. The exemplary processed data approximates an exponential decay curve characteristic for the luminescent lifetime of the luminescent label in the target volume. Because luminescent label # has a shorter luminescent lifetime than luminescent label *, the processed data for Stage B has fewer counts at longer time durations, while the processed data for Stage D has relatively more counts at longer time durations.

The example experiment of FIG. 2 would identify the first two nucleotides added to the complementary strand as CA. For DNA, the sequence of the target strand immediately after the region is hybridized to the primer would thus be identified as GT. In this example the nucleotides C and A could be distinguished from amongst the plurality of C, G, T, and A, based on luminescent lifetime alone. In some embodiments, other properties, such as the luminescent intensity or the wavelength of the absorbed or emitted photons may be necessary to distinguish one or more particular nucleotide.

Signals emitted upon the incorporation of nucleotides can be stored in memory and processed at a later point in time to determine the sequence of the target nucleic acid template. This may include comparing the signals to a reference signals to determine the identities of the incorporated nucleotides as a function of time. Alternatively or in addition to, signal emitted upon the incorporation of nucleotide can be collected and processed in real time (e.g., upon nucleotide incorporation) to determine the sequence of the target nucleic acid template in real time.

A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate, which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, that include luminescent labels or markers (e.g., fluorophores).

A nucleoside polyphosphate can have ‘n’ phosphate groups, where ‘n’ is a number that is greater than or equal to 2, 3, 4, 5, 6, 7, 8, 9, or 10. Examples of nucleoside polyphosphates include nucleoside diphosphate and nucleoside triphosphate. A nucleotide can be a terminal phosphate labeled nucleoside, such as a terminal phosphate labeled nucleoside polyphosphate. Such label can be a luminescent (e.g., fluorescent or chemiluminescent) label, a fluorogenic label, a colored label, a chromogenic label, a mass tag, an electrostatic label, or an electrochemical label. A label (or marker) can be coupled to a terminal phosphate through a linker. The linker can include, for example, at least one or a plurality of hydroxyl groups, sulfhydryl groups, amino groups or haloalkyl groups, which may be suitable for forming, for example, a phosphate ester, a thioester, a phosphoramidate or an alkyl phosphonate linkage at the terminal phosphate of a natural or modified nucleotide. A linker can be cleavable so as to separate a label from the terminal phosphate, such as with the aid of a polymerization enzyme. Examples of nucleotides and linkers are provided in U.S. Pat. No. 7,041,812, which is incorporated herein by reference.

A nucleotide (e.g., a nucleotide polyphosphate) can comprise a methylated nucleobase. For example, a methylated nucleotide can be a nucleotide that comprises one or more methyl groups attached to the nucleobase (e.g., attached directly to a ring of the nucleobase, attached to a substituent of a ring of the nucleobase). Exemplary methylated nucleobases include 1-methylthymine, 1-methyluracil, 3-methyluracil, 3-methylcytosine, 5-methylcytosine, 1-methyladenine, 2-methyladenine, 7-methyladenine, N6-methyladenine, N₆,N₆-dimethyladenine, 1-methylguanine, 7-methylguanine, N₂-methylguanine, and N₂,N₂-dimethylguanine.

The term “primer,” as used herein, generally refers to a nucleic acid molecule (e.g., an oligonucleotide), which can include a sequence comprising A, C, G, T and/or U, or variants or analogs thereof. A primer can be a synthetic oligonucleotide comprising DNA, RNA, PNA, or variants or analogs thereof. A primer can be designed such that its nucleotide sequence is complementary to a target strand, or the primer can comprise a random nucleotide sequence. In some embodiments, a primer can comprise a tail (e.g., a poly-A tail, an index adaptor, a molecular barcode, etc.). In some embodiments, a primer can comprise 5 to 15 bases, 10 to 20 bases, 15 to 25 bases, 20 to 30 bases, 25 to 35 bases, 30 to 40 bases, 35 to 45 bases, 40 to 50 bases, 45 to 55 bases, 50 to 60 bases, 55 to 65 bases, 60 to 70 bases, 65 to 75 bases, 70 to 80 bases, 75 to 85 bases, 80 to 90 bases, 85 to 95 bases, 90 to 100 bases, 95 to 105 bases, 100 to 150 bases, 125 to 175 bases, 150 to 200 bases, or more than 200 bases.

### Peptide Sequencing

In some embodiments, aspects of the disclosure can be used to assay biological samples, for example to determine the sequence of one or more polypeptides in the sample and/or to determine the presence or absence of one or more variants (e.g., one or more amino acid substitutions in a polypeptide of interest) in the sample. In some embodiments, the compounds described herein may be subjected to peptide sequencing (also referred to as “polypeptide sequencing”) by detecting single molecule binding interactions during a peptide degradation process. In some embodiments, the peptide of interest is covalently or non-covalently attached to a surface.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (and, for instance, determining the relative position of one or more amino acid types without determining the identity of each amino acid in the polypeptide). In some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some aspects, the methods for sequencing of a peptide provided herein may be performed by identifying one or more types of amino acids of a polypeptide. In some embodiments, one or more amino acids (e.g., terminal amino acids and/or internal amino acids) of the polypeptide are labeled (e.g., directly or indirectly, for example using a molecule interacting with the polypeptide biomolecule such as an amino acid recognition molecule) and the relative positions of the labeled amino acids in the polypeptide are determined. In some embodiments, the relative positions of amino acids in a polypeptide are determined using a series of amino acid labeling and cleavage steps. However, in some embodiments, the relative position of labeled amino acids in a polypeptide can be determined without removing amino acids from the polypeptide but by translocating a labeled polypeptide through a pore (e.g., a protein channel) and detecting a signal (e.g., a FRET signal) from the labeled amino acid(s) during translocation through the pore in order to determine the relative position of the labeled amino acids in the polypeptide molecule.

In some embodiments, the identity of a terminal amino acid (e.g., an N-terminal or a C-terminal amino acid) is assessed after which the terminal amino acid is removed and the identity of the next amino acid at the terminus is assessed, and this process is repeated until a plurality of successive amino acids in the polypeptide are assessed. In some embodiments, assessing the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

However, in some embodiments assessing the identity of a terminal amino acid type can comprise determining a subset of potential amino acids that can be present at the terminus of the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the polypeptide (e.g., using a binding agent that binds to a specified subset of two or more terminal amino acids). In some embodiments, assessing the identity of a terminal amino acid type comprises determining that an amino acid comprises a post-translational modification.

In some embodiments, methods provided herein comprise contacting a polypeptide with a labeled amino acid recognition molecule (also referred to herein as an affinity reagent) that selectively binds one type of terminal amino acid. In some embodiments, a labeled affinity reagent selectively binds one type of terminal amino acid over other types of terminal amino acids. In some embodiments, a labeled affinity reagent selectively binds one type of terminal amino acid over an internal amino acid of the same type. In yet other embodiments, a labeled affinity reagent selectively binds one type of amino acid at any position of a polypeptide, e.g., the same type of amino acid as a terminal amino acid and an internal amino acid.

In some embodiments, polypeptide sequencing can proceed by contacting the polypeptide with one or more amino acid recognition molecules that associate with one or more types of terminal amino acids. In some embodiments, a labeled amino acid recognition molecule interacts with polypeptide by associating with the terminal amino acid.

Accordingly, in some embodiments, the one or more types of amino acids are identified by detecting luminescence of one or more labeled amino acid recognition molecules that selectively bind the one or more types of amino acids. In some embodiments, methods provided herein comprise contacting a polypeptide with one or more labeled amino acid recognition molecules (or affinity reagents) that selectively bind one or more types of terminal amino acids. As an illustrative and non-limiting example, where four labeled affinity reagents are used in a method of the application, any one reagent selectively binds one type of terminal amino acid that is different from another type of amino acid to which any of the other three selectively binds (e.g., a first reagent binds a first type, a second reagent binds a second type, a third reagent binds a third type, and a fourth reagent binds a fourth type of terminal amino acid). For the purposes of this discussion, one or more labeled affinity reagents in the context of a method described herein may be alternatively referred to as a set of labeled affinity reagents.

In some embodiments, a set of labeled affinity reagents comprises at least one and up to six labeled affinity reagents. For example, in some embodiments, a set of labeled affinity reagents comprises one, two, three, four, five, or six labeled affinity reagents. In some embodiments, a set of labeled affinity reagents comprises ten or fewer labeled affinity reagents. In some embodiments, a set of labeled affinity reagents comprises eight or fewer labeled affinity reagents. In some embodiments, a set of labeled affinity reagents comprises six or fewer labeled affinity reagents. In some embodiments, a set of labeled affinity reagents comprises four or fewer labeled affinity reagents. In some embodiments, a set of labeled affinity reagents comprises three or fewer labeled affinity reagents. In some embodiments, a set of labeled affinity reagents comprises two or fewer labeled affinity reagents. In some embodiments, a set of labeled affinity reagents comprises four labeled affinity reagents. In some embodiments, a set of labeled affinity reagents comprises at least two and up to twenty (e.g., at least two and up to ten, at least two and up to eight, at least four and up to twenty, at least four and up to ten) labeled affinity reagents. In some embodiments, a set of labeled affinity reagents comprises more than twenty (e.g., 20 to 25, 20 to 30) affinity reagents. It should be appreciated, however, that any number of affinity reagents may be used in accordance with a method of the application to accommodate a desired use.

In accordance with the application, in some embodiments, one or more types of amino acids are identified by detecting luminescence of a labeled affinity reagent (e.g., an amino acid recognition molecule comprising a luminescent label). In some embodiments, a labeled affinity reagent comprises an affinity reagent that selectively binds one type of amino acid and a luminescent label having a luminescence that is associated with the affinity reagent. In this way, the luminescence (e.g., luminescence lifetime, luminescence intensity, and other luminescence properties described elsewhere herein) may be associated with the selective binding of the affinity reagent to identify an amino acid of a polypeptide. In some embodiments, a plurality of types of labeled affinity reagents may be used in a method according to the application, wherein each type comprises a luminescent label having a luminescence that is uniquely identifiable from among the plurality.

In some embodiments, one or more types of amino acids are identified by detecting one or more electrical characteristics of a labeled affinity reagent. In some embodiments, a labeled affinity reagent comprises an affinity reagent that selectively binds one type of amino acid and a conductivity label that is associated with the affinity reagent. In this way, the one or more electrical characteristics (e.g., charge, current oscillation color, and other electrical characteristics) may be associated with the selective binding of the affinity reagent to identify an amino acid of a polypeptide. In some embodiments, a plurality of types of labeled affinity reagents may be used in a method according to the application, wherein each type comprises a conductivity label that produces a change in an electrical signal (e.g., a change in conductance, such as a change in amplitude of conductivity and conductivity transitions of a characteristic pattern) that is uniquely identifiable from among the plurality. In some embodiments, the plurality of types of labeled affinity reagents each comprises a conductivity label having a different number of charged groups (e.g., a different number of negatively and/or positively charged groups). Accordingly, in some embodiments, a conductivity label is a charge label. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

In some embodiments, an affinity reagent may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide. In some embodiments, desirable properties include an ability to bind selectively and with low affinity (e.g., with a K_{D} of about 50 nM or higher, for example, between about 50 nM and about 50 μM, between about 100 nM and about 10 μM, between about 500 nM and about 50 μM) to more than one type of amino acid. For example, in some aspects, the application provides methods of sequencing by detecting reversible binding interactions during a polypeptide degradation process. Advantageously, such methods may be performed using an affinity reagent that reversibly binds with low affinity to more than one type of amino acid (e.g., a subset of amino acid types).

In some embodiments, polypeptide sequencing comprises providing an amino acid recognition molecule that binds a polypeptide biomolecule of interest, wherein the recognition molecule is immobilized to a surface of a solid support (e.g., attached to a bottom surface of a sample well) through a coupling moiety or a coupling moiety-copolymer conjugate. In some embodiments, the coupling moiety-copolymer conjugate is formed by a non-covalent linkage between a copolymer and a biotin coupling moiety or a streptavidin coupling moiety conjugated to the surface of a well (e.g., functionalized in accordance with aspects of the disclosure) (see, e.g., FIG. 8).

In some embodiments, the recognition molecule is immobilized to the surface through a coupling moiety at one terminal end such that the other terminal end is free for detecting a terminal amino acid of the polypeptide in the sample. Accordingly, in some embodiments, the non-immobilized (e.g., free) end of the recognition molecules of the disclosure preferentially interact with terminal amino acids of the polypeptide of interest.

In other aspects, sequencing in accordance with the disclosure may involve immobilizing a polypeptide on a surface of a substrate (e.g., of a solid support, for example a chip, for example an integrated device as described herein). In some embodiments, a polypeptide may be immobilized on a surface of a sample well (e.g., on a bottom surface of a sample well) on a substrate. In some embodiments, the N-terminal amino acid of the polypeptide is immobilized (e.g., attached to the surface). In some embodiments, the C-terminal amino acid of the polypeptide is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this application. In some embodiments, a plurality of polypeptides are attached to a plurality of sample wells (e.g., with one polypeptide attached to a surface, for example a bottom surface, of each sample well), for example in an array of sample wells on a substrate. Accordingly, in some embodiments, the non-immobilized (e.g., free) terminus end of an immobilized polypeptide preferentially interacts with the amino acid recognition molecule.

In some embodiments, the method further comprises identifying the amino acid (i.e., a terminal or internal amino acid) of the polypeptide by detecting the labeled amino acid recognition molecule. In some embodiments, detecting comprises detecting a luminescence from labeled amino acid recognition molecule. In some embodiments, the luminescence is uniquely associated with the labeled amino acid recognition molecule, and the luminescence is thereby associated with the type of amino acid to which the labeled amino acid recognition molecule selectively binds. As such, in some embodiments, the type of amino acid is identified by determining one or more luminescence properties of the labeled amino acid recognition molecule.

In some embodiments, the polypeptide biomolecule is degraded by a cleaving reagent that removes one or more amino acids from the terminus of the single polypeptide molecule. In some embodiments, the methods further comprise detecting a signal indicative of association of the cleaving reagent with the terminus. In some embodiments, the cleaving reagent comprises a detectable label (e.g., a luminescent label, a conductivity label). In such embodiments, the single polypeptide molecule is immobilized to a surface. In some embodiments, the single polypeptide molecule is immobilized to the surface through a terminal end distal to the terminus with which the one or more terminal amino acid recognition molecules interact.

In other embodiments, polypeptide sequencing proceeds by removing the terminal amino acid by contacting the polypeptide with an exopeptidase that binds and cleaves the terminal amino acid of the polypeptide. Upon removal of the terminal amino acid by exopeptidase, polypeptide sequencing proceeds by subjecting the polypeptide (having n−1 amino acids) to additional cycles of terminal amino acid recognition and cleavage. In some embodiments, these steps may occur in the same reaction mixture, e.g., as in a dynamic peptide sequencing reaction. In some embodiments, these steps may be carried out using other methods known in the art, such as peptide sequencing by Edman degradation.

In some embodiments, dynamic polypeptide sequencing is carried out in real-time by evaluating binding interactions of terminal amino acids with labeled amino acid recognition molecules and a cleaving reagent (e.g., an exopeptidase). A labeled amino acid recognition molecule may associate with (e.g., bind to) and dissociate from a terminal amino acid, which may give rise to a series of pulses in signal output which may be used to identify the terminal amino acid. In some embodiments, the series of pulses provide a pulsing pattern (e.g., a characteristic pattern) which may be diagnostic of the identity of the corresponding terminal amino acid.

In some embodiments, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). It should be appreciated that, in some embodiments, smaller differences in mean pulse duration between different characteristic patterns may require a greater number of pulse durations within each characteristic pattern to distinguish one from another with statistical confidence.

Each signal pulse of a characteristic pattern is separated from another signal pulse of the characteristic pattern by an interpulse duration. In some embodiments, the interpulse duration is characteristic of an association rate of binding. In some embodiments, a change in magnitude can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

In some aspects, the disclosure provides methods of immobilizing a peptide, or an amino acid recognition molecule, to a surface by attaching any one of the compounds described herein to a surface of a sample well. In some embodiments, the surface is functionalized with a coupling moiety (or functional moiety) configured for attachment (e.g., covalent attachment) to a functionalized terminal end of a peptide. In some embodiments, the methods comprise immobilizing a single peptide to a surface of each of a plurality of sample wells. In some embodiments, confining a single peptide per sample well is advantageous for single molecule detection methods, e.g., single molecule peptide sequencing.

In some embodiments, peptide comprising functionalized terminal end is contacted with complementary coupling moiety. In some embodiments, functionalized terminal end and coupling moiety comprise partner click chemistry handles, e.g., which form a covalent linkage between peptide and the sample well surface. Suitable click chemistry handles are described herein. In some embodiments, functionalized terminal end and coupling moiety comprise non-covalent binding partners, e.g., which form a non-covalent linkage between peptide and the sample well surface. Examples of non-covalent binding partners include protein-protein binding partners (e.g., barnase and barstar), and protein-ligand binding partners (e.g., biotin and streptavidin).

### Characteristics of Luminescent Labels

As described herein, a luminescent label or labeled molecule is a molecule that absorbs one or more photons and may subsequently emit one or more photons after one or more time durations. The luminescence of the molecule is described by several parameters, including but not limited to luminescent lifetime, absorption spectra, emission spectra, luminescent quantum yield, and luminescent intensity. The terms absorption and excitation are used interchangeably throughout the disclosure. A typical luminescent molecule may absorb, or undergo excitation by, light at multiple wavelengths. Excitation at certain wavelengths or within certain spectral ranges may relax by a luminescent emission event, while excitation at certain other wavelengths or spectral ranges may not relax by a luminescent emission event. In some embodiments, a luminescent molecule is only suitably excited for luminescence at a single wavelength or within a single spectral range. In some embodiments, a luminescent molecule is suitably excited for luminescence at two or more wavelengths or within two or more spectral ranges. In some embodiments, a molecule is identified by measuring the wavelength of the excitation photon or the absorption spectrum.

The emitted photon from a luminescent emission event will emit at a wavelength within a spectral range of possible wavelengths. Typically, the emitted photon has a longer wavelength (e.g., has less energy or is red-shifted) compared to the wavelength of the excitation photon. In certain embodiments, a molecule is identified by measuring the wavelength of an emitted photon. In certain embodiments, a molecule is identified by measuring the wavelength of a plurality of emitted photon. In certain embodiments, a molecule is identified by measuring the emission spectrum.

Luminescent lifetime refers to the time duration between an excitation event and an emission event. In some embodiments, luminescent lifetime is expressed as the constant in an equation of exponential decay. In some embodiments, wherein there are one or more pulse events delivering excitation energy, the time duration is the time between the pulse and the subsequent emission event.

“Determining a luminescent lifetime” of a molecule can be performed using any suitable method (e.g., by measuring the lifetime using a suitable technique or by determining time-dependent characteristics of emission). In some embodiments, determining the luminescent lifetime of a molecule comprises determining the lifetime relative to one or more molecules (e.g., different luminescently labeled molecules in a sequencing reaction). In some embodiments, determining the luminescent lifetime of a molecule comprises determining the lifetime relative to a reference. In some embodiments, determining the luminescent lifetime of a molecule comprises measuring the lifetime (e.g., luminescent lifetime). In some embodiments, determining the luminescent lifetime of a molecule comprises determining one or more temporal characteristics that are indicative of lifetime. In some embodiments, the luminescent lifetime of a molecule can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescent lifetime of a single molecule can be distinguished from a plurality of molecules having different luminescent lifetimes based on the distribution of photon arrival times measured with respect to an excitation pulse.

It should be appreciated that a luminescent lifetime of a labeled molecule is indicative of the timing of photons emitted after the molecule reaches an excited state and the molecule can be distinguished by information indicative of the timing of the photons. Some embodiments may include distinguishing a labeled molecule from a plurality of molecules based on the label's luminescent lifetime by measuring times associated with photons emitted by the label. The distribution of times may provide an indication of the luminescent lifetime which may be determined from the distribution. In some embodiments, the labeled molecule is distinguishable from the plurality of molecules based on the distribution of times, such as by comparing the distribution of times to a reference distribution corresponding to a known molecule. In some embodiments, a value for the luminescent lifetime is determined from the distribution of times.

Luminescent quantum yield refers to the fraction of excitation events at a given wavelength or within a given spectral range that lead to an emission event and is typically less than 1. In some embodiments, the luminescent quantum yield of a labeled molecule described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a labeled molecule is identified by determining or estimating the luminescent quantum yield.

As used herein for labeled molecules, luminescent intensity refers to the number of emitted photons per unit time that are emitted by a molecule which is being excited by delivery of a pulsed excitation energy. In some embodiments, the luminescent intensity refers to the detected number of emitted photons per unit time that are emitted by the molecule which is being excited by delivery of a pulsed excitation energy, and are detected by a particular sensor or set of sensors.

The luminescent lifetime, luminescent quantum yield, and luminescent intensity may each vary for a given labeled molecule under different conditions. In some embodiments, a labeled molecule will have a different observed luminescent lifetime, luminescent quantum yield, or luminescent intensity than for an ensemble of the molecules. In some embodiments, a labeled molecule confined in a sample well will have a different observed luminescent lifetime, luminescent quantum yield, or luminescent intensity than for labeled molecules not confined in a sample well. In some embodiments, a luminescent label or luminescent molecule conjugated to another molecule will have a different luminescent lifetime, luminescent quantum yield, or luminescent intensity than the luminescent label or luminescent molecule not conjugated to another molecule. In some embodiments, a labeled molecule interacting with a biomolecule that is a macromolecular complex (e.g., protein complex or nucleic acid polymerase complex) will have different luminescent lifetime, luminescent quantum yield, or luminescent intensity than a labeled molecule not interacting with a macromolecular complex.

In certain embodiments, a labeled molecule described in the disclosure absorbs one photon and emits one photon after a time duration. In some embodiments, the luminescent lifetime of a labeled molecule can be determined or estimated by measuring the time duration. In some embodiments, the luminescent lifetime of a molecule can be determined or estimated by measuring a plurality of time durations for multiple pulse events and emission events. In some embodiments, the luminescent lifetime of a molecule can be differentiated amongst the luminescent lifetimes of a plurality of types of molecules by measuring the time duration. In some embodiments, the luminescent lifetime of a molecule can be differentiated amongst the luminescent lifetimes of a plurality of types of molecules by measuring a plurality of time durations for multiple pulse events and emission events. In certain embodiments, a molecule is identified or differentiated amongst a plurality of types of molecules by determining or estimating the luminescent lifetime of the molecule. In certain embodiments, a molecule is identified or differentiated amongst a plurality of types of molecules by differentiating the luminescent lifetime of the molecule amongst a plurality of the luminescent lifetimes of a plurality of types of molecules.

In certain embodiments, the luminescent emission event is a fluorescence. In certain embodiments, the luminescent emission event is a phosphorescence. As used herein, the term “luminescence” encompasses all luminescent events including both fluorescence and phosphorescence.

In one aspect, the disclosure provides a method of determining the luminescent lifetime (or intensity) of a single luminescent molecule comprising: providing the luminescent molecule in a target volume; delivering a plurality of pulses of an excitation energy to a vicinity of the target volume; and detecting a plurality of luminescences from the luminescent molecule. In some embodiments, the method further comprises evaluating the distribution of the plurality of time durations between each pair of pulses and luminescences. In some embodiments, the method further comprises immobilizing the single luminescent molecule in the target volume.

In some aspects, the disclosure provides a method of determining the luminescent lifetime (or intensity) of a plurality of molecules comprising: providing a plurality of luminescent molecules in a target volume; delivering a plurality of pulses of an excitation energy to a vicinity of the target volume; and detecting a plurality of luminescences from the luminescent molecules. In some embodiments, the method further comprises evaluating the distribution of the plurality of time durations between each pair of pulses and luminescences. In some embodiments, the method further comprises immobilizing the luminescent molecules in the target volume. In some embodiments, the plurality consists of between 2 and about 10 molecules, between about 10 and about 100 molecules, or between about 100 and about 1000 molecules. In some embodiments, the plurality consists of between about 1000 and about 10⁶ molecules, between about 10⁶ and about 10⁹ molecules, between about 10⁹ and about 10¹² molecules, between about 10¹² and about 10¹⁵ molecules, or between about 10¹⁵ and about 10¹⁸ molecules. In some embodiments, all molecules of the plurality are the same type of molecule.

### Excitation Energy

In one aspect of methods described herein, one or more excitation energies, e.g., in the form of laser light pulses, is used to excite the luminescent labels of the molecules to be identified or distinguished (e.g., during a sequencing reaction). In some embodiments, an excitation energy is in the visible spectrum. In some embodiments, an excitation energy is in the ultraviolet spectrum. In some embodiments, an excitation energy is in the infrared spectrum. In some embodiments, one excitation energy is used to excite the luminescently labeled molecules. In some embodiments, two excitation energies are used to excite the luminescently labeled molecules. In some embodiments, three or more excitation energies are used to excite the luminescently labeled molecules. In some embodiments, each luminescently labeled molecule is excited by only one of the delivered excitation energies. In some embodiments, a luminescently labeled molecule is excited by two or more of the delivered excitation energies. In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments a spectral range has a range of between about 5 nm and about 10 nm, between about 10 nm and about 50 nm, or between about 50 nm and about 100 nm.

In certain embodiments, excitation energy is delivered as a pulse of light. In certain embodiments, excitation energy is delivered as a plurality of pulses of light. In certain embodiments, two or more excitation energies are used to excite the luminescently labeled molecules. In some embodiments, each excitation energy is delivered at the same time (e.g., in each pulse). In some embodiments, each excitation energy is delivered at different times (e.g., in separate pulses of each energy). The different excitation energies may be delivered in any pattern sufficient to allow detection of luminescence from the target molecules. In some embodiments, two excitation energies are delivered in each pulse. In some embodiments, a first excitation energy and a second excitation energy are delivered in alternating pulses. In some embodiments, a first excitation energy is delivered in a series of sequential pulses, and a second excitation energy is delivered in a subsequent series of sequential pulses, or an alternating pattern of such series.

In certain embodiments, the frequency of pulses of light is selected based on the luminescent properties of the luminescently labeled molecule. In certain embodiments, the frequency of pulses of light is selected based on the luminescent properties of a plurality of luminescently labeled nucleotides or amino acid recognition molecules. In certain embodiments, the frequency of pulses of light is selected based on the luminescent lifetime of a plurality of luminescently labeled nucleotides or amino acid recognition molecules. In some embodiments, the frequency is selected so that the gap between pulses is longer than the luminescent lifetimes of one or more luminescently labeled nucleotides or amino acid recognition molecules. In some embodiments, the frequency is selected based on the longest luminescent lifetime of the plurality of luminescently labeled nucleotides or amino acid recognition molecules. For example, if the luminescent lifetimes of the four luminescently labeled nucleotides are 0.25, 0.5, 1.0, and 1.5 ns, the frequency of pulses of light may be selected so that the gap between pulses exceeds 1.5 ns. In some embodiments, the gap is between about two times and about ten times, between about ten times and about 100 times, or between about 100 times and about 1000 times longer than the luminescent lifetime of one or more luminescently labeled molecules being excited. In some embodiments, the gap is about 10 times longer than the luminescent lifetime of one or more luminescently labeled molecules being excited. In some embodiments, the gap is between about 0.01 ns and about 0.1 ns, between about 1 ns and about 5 ns, between about 5 ns and about 15 ns, between about 15 ns and about 25 ns, or between about 25 ns and about 50 ns. In some embodiments, the gap is selected such that there is a 50%, 75%, 90%, 95%, or 99% probability that the molecules excited by the pulse will luminescently decay or that the excited state will relax by another mechanism.

In certain embodiments, wherein there are multiple excitation energies, the frequency of the pulses for each excitation energy is the same. In certain embodiments, wherein there are multiple excitation energies, the frequencies of the pulses for each excitation energy is different. For example, if a red laser is used to excite luminescent molecules with lifetimes of 0.2 and 0.5 ns, and a green laser is used to excite luminescent molecules with lifetimes of 5 ns and 7 ns, the gap after each red laser pulse may be shorter (e.g., 5 ns) than the gap after each green laser pulse (e.g., 20 ns).

In certain embodiments, the frequency of pulsed excitation energies is selected based on the chemical process being monitored. For a sequencing reaction the frequency may be selected such that a number of pulses are delivered sufficient to allow for detection of a sufficient number of emitted photons to be detected. A sufficient number, in the context of detected photons, refers to a number of photons necessary to identify or distinguish the luminescently labeled nucleotide or amino acid recognition molecule from the plurality of luminescently labeled nucleotides or amino acid recognition molecules. For example, a DNA polymerase may incorporate an additional nucleotide once every 20 milliseconds on average. The time that a luminescently labeled nucleotide interacts with the complex may be about 10 milliseconds, and the time between when the luminescent marker is cleaved and the next luminescently labeled nucleotide begins to interact may be about 10 milliseconds. The frequency of the pulsed excitation energy could then be selected to deliver sufficient pulses over 10 milliseconds such that a sufficient number of emitted photons are detected during the 10 millisecond when the luminescently labeled nucleotide is being incorporated. For example, at a frequency of 100 MHz, there will be 1 million pulses in 10 milliseconds (the approximate length of the incorporation event). If 0.10% of these pulses leads to a detected photon there will be 1,000 luminescent data points that can be analyzed to determine the identity of the luminescently labeled nucleotide being incorporated. Any of the above values are non-limiting. In some embodiments incorporation events may take between 1 ms and 20 ms, between 20 ms and 100 ms, or between 100 ms and 500 ms. In some embodiments, in which multiple excitation energies are delivered in separately timed pulses the luminescently labeled nucleotide may only be excited by a portion of the pulses. In some embodiments, the frequency and pattern of the pulses of multiple excitation energies is selected such that the number of pulses is sufficient to excite any one of the plurality of luminescently labeled nucleotides to allow for a sufficient number of emitted photons to be detected.

In some embodiments, the frequency of pulses is between about 1 MHz and about 10 MHz. In some embodiments, the frequency of pulses is between about 10 MHz and about 100 MHz. In some embodiments, the frequency of pulses is between about 100 MHz and about 1 GHz. In some embodiments, the frequency of pulses is between about 50 MHz and about 200 MHz. In some embodiments, the frequency of pulses is about 100 MHz. In some embodiments, the frequency is stochastic.

In certain embodiments, the excitation energy is between about 500 nm and about 700 nm. In some embodiments, the excitation energy is between about 500 nm and about 600 nm, or about 600 nm and about 700 nm. In some embodiments, the excitation energy is between about 500 nm and about 550 nm, between about 550 nm and about 600 nm, between about 600 nm and about 650 nm, or between about 650 nm and about 700 nm.

In certain embodiments, a method described herein comprises delivery of two excitation energies. In some embodiments, the two excitation energies are separated by between about 5 nm and about 20 nm, between about 20 nm and about 40 nm, between about 40 nm and about 60 nm, between about 60 nm and about 80 nm, between about 80 nm and about 100 nm, between about 100 nm and about 150 nm, between about 150 nm and about 200 nm, between about 200 nm and about 400 nm, or between at least about 400 nm. In some embodiments, the two excitation energies are separated by between about 20 nm and about 80 nm, or between about 80 nm and about 160 nm.

When an excitation energy is referred to as being in a specific range, the excitation energy may comprise a single wavelength, such that the wavelength is between or at the endpoints of the range, or the excitation energy may comprise a spectrum of wavelengths with a maximum intensity, such that the maximum intensity is between or at the endpoints of the range.

In certain embodiments, the first excitation energy is in the range of 450 nm to 500 nm and the second excitation energy is in the range of 500 nm to 550 nm, 550 nm to 600 nm, 600 nm to 650 nm, or 650 nm to 700 nm. In certain embodiments, the first excitation energy is in the range of 500 nm to 550 nm and the second excitation energy is in the range of 450 nm to 500 nm, 550 nm to 600 nm, 600 nm to 650 nm, or 650 nm to 700 nm. In certain embodiments, the first excitation energy is in the range of 550 nm to 600 nm and the second excitation energy is in the range of 450 nm to 500 nm, 500 nm to 550 nm, 600 nm to 650 nm, or 650 nm to 700 nm. In certain embodiments, the first excitation energy is in the range of 600 nm to 650 nm and the second excitation energy is in the range of 450 nm to 500 nm, 500 nm to 550 nm, 550 nm to 600 nm, or 650 nm to 700 nm. In certain embodiments, the first excitation energy is in the range of 650 nm to 700 nm and the second excitation energy is in the range of 450 nm to 500 nm, 500 nm to 550 nm, 550 nm to 600 nm, or 600 nm to 650 nm. In certain embodiments, the first excitation energy is in the range of 470 nm to 510 nm and the second excitation energy is in the range of 550 nm to 580 nm. In certain embodiments, the first excitation energy is in the range of 470 nm to 510 nm and the second excitation energy is in the range of 580 nm to 620 nm. In certain embodiments, the first excitation energy is in the range of 470 nm to 510 nm and the second excitation energy is in the range of 620 nm to 670 nm. In certain embodiments, the first excitation energy is in the range of 510 nm to 550 nm and the second excitation energy is in the range of 550 nm to 580 nm. In certain embodiments, the first excitation energy is in the range of 510 nm to 550 nm and the second excitation energy is in the range of 580 nm to 620 nm. In certain embodiments, the first excitation energy is in the range of 510 nm to 550 nm and the second excitation energy is in the range of 620 nm to 670 nm. In certain embodiments, the first excitation energy is in the range of 550 nm to 580 nm and the second excitation energy is in the range of 580 nm to 620 nm. In certain embodiments, the first excitation energy is in the range of 550 nm to 580 nm and the second excitation energy is in the range of 620 nm to 670 nm. In certain embodiments, the first excitation energy is in the range of 580 nm to 620 nm and the second excitation energy is in the range of 620 nm to 670 nm.

### Luminescent Labels

The terms luminescent label and luminescent marker are used interchangeably throughout and relate to molecules comprising one or more luminescent molecules. In certain embodiments, the incorporated molecule is a luminescent molecule, e.g., without attachment of a distinct luminescent label. Typical nucleotide and amino acids are not luminescent, or do not luminesce within suitable ranges of excitation and emission energies. In certain embodiments, the incorporated molecule comprises a luminescent label. In certain embodiments, the incorporated molecule is a luminescently labeled nucleotide. In certain embodiments, the incorporated molecule is a luminescently labeled amino acid recognition molecule. In some embodiments, a luminescently labeled nucleotide comprises a nucleotide and a luminescent label. In some embodiments, the luminescent label is a fluorophore.

In certain embodiments, the luminescent label remain attached to the incorporated molecule. In certain embodiments, the luminescent label are cleaved from the molecule during or after the process of incorporation.

In certain embodiments, the luminescent label is a cyanine dye, or an analog thereof. In some embodiments, the cyanine dye is of formula:

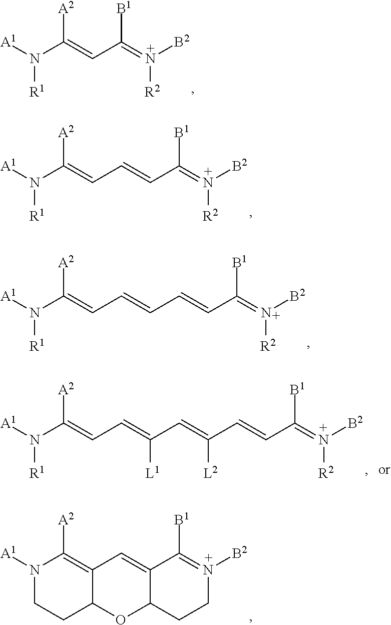

or a salt, stereoisomer, or tautomer thereof, wherein:
A¹ and A² are joined to form an optionally substituted, aromatic or non-aromatic, monocyclic or polycyclic, heterocyclic ring;
B¹ and B² are joined to form an optionally substituted, aromatic or non-aromatic, monocyclic or polycyclic, heterocyclic ring;
each of R¹ and R² is independently hydrogen, optionally substituted alkyl; and
each of L¹ and L² is independently hydrogen, optionally substituted alkyl, or L¹ and L² are joined to form an optionally substituted, aromatic or non-aromatic, monocyclic or polycyclic, carbocyclic ring.

In certain embodiments, the luminescent label is a rhodamine dye, or an analog thereof. In some embodiments, the rhodamine dye is of formula:

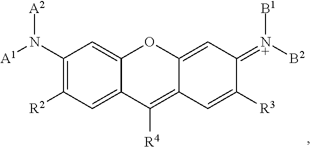

or a salt, stereoisomer, or tautomer thereof, wherein:
each of A¹ and A² is independently hydrogen, optionally substituted alkyl, optionally substituted aromatic or non-aromatic heterocyclyl, optionally substituted aromatic or non-aromatic carbocyclyl, or optionally substituted carbonyl, or A¹ and A² are joined to form an optionally substituted, aromatic or non-aromatic, monocyclic or polycyclic, heterocyclic ring;
each of B¹ and B² is independently hydrogen, optionally substituted alkyl, optionally substituted, aromatic or non-aromatic heterocyclyl, optionally substituted, aromatic or non-aromatic carbocyclyl, or optionally substituted carbonyl, or B¹ and B² are joined to form an optionally substituted, aromatic or non-aromatic, monocyclic or polycyclic, heterocyclic ring;
each of R² and R³ is independently hydrogen, optionally substituted alkyl, optionally substituted aryl, or optionally substituted acyl; and
R⁴ is hydrogen, optionally substituted alkyl, optionally substituted, optionally substituted aromatic or non-aromatic heterocyclyl, optionally substituted aromatic or non-aromatic carbocyclyl, or optionally substituted carbonyl.

In some embodiments, R⁴ is optionally substituted phenyl. In some embodiments, R⁴ is optionally substituted phenyl, wherein at least one substituent is optionally substituted carbonyl. In some embodiments, R⁴ is optionally substituted phenyl, wherein at least one substituent is optionally substituted sulfonyl.

In some embodiments, the luminescent label comprises an aromatic or heteroaromatic compound and can be a pyrene, anthracene, naphthalene, acridine, stilbene, indole, benzindole, oxazole, carbazole, thiazole, benzothiazole, phenanthridine, phenoxazine, porphyrin, quinoline, ethidium, benzamide, cyanine, carbocyanine, salicylate, anthranilate, coumarin, fluoroscein, rhodamine or other like compound. Exemplary dyes include xanthene dyes, such as fluorescein or rhodamine dyes, including 5-carboxyfluorescein (FAM), 27′-dimethoxy-4′5′-dichloro-6-carboxyfluorescein (JOE), tetrachlorofluorescein (TET), 6-carboxyrhodamine (R6G), N,N,N′,N′-tetramethyl-6-carboxyrhodamine (TAMRA), 6-carboxy-X-rhodamine (ROX). Exemplary dyes also include naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Other exemplary dyes include coumarins, such as 3-phenyl-7-isocyanatocoumarin; acridines, such as 9-isothiocyanatoacridine and acridine orange; N-(p-(2-benzoxazolyl)phenyl)maleimide; cyanines, such as indodicarbocyanine 3 (Cy®3), (2Z)-2-[(E)-3-[3-(5-carboxypentyl)-1,1-dimethyl-6,8-disulfobenzo[e]indol-3-ium-2-yl]prop-2-enylidene]-3-ethyl-1,1-dimethyl-8-(trioxidanylsulfanyl)benzo[e]indole-6-sulfonate (Cy®3.5), 2-{2-[(2,5-dioxopyrrolidin-1-yl)oxy]-2-oxoethyl}-16,16,18,18-tetramethyl-6,7,7a,8a,9,10,16,18-octahydrobenzo[2″,3″]indolizino[8″,7″:5′,6′]pyrano[3′,2′:3,4]pyrido[1,2-a]indol-5-ium-14-sulfonate (Cy®3B), indodicarbocyanine 5 (Cy®5), indodicarbocyanine 5.5 (Cy®5.5), 3-(-carboxy-pentyl)-3′-ethyl-5,5′-dimethyloxacarbocyanine (CyA); 1H,5H,11H,15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17-octahydro-inner salt (TR or Texas Red®); BODIPY® dyes; benzoxazoles; stilbenes; pyrenes; and the like.

In some embodiments, at least two luminescently labeled molecules absorb a first excitation energy, wherein at least one of the labels comprises a cyanine dye, or analog thereof. In some embodiments, at least two luminescently labeled molecules absorb a second excitation energy, wherein at least one of the labels comprises a cyanine dye, or analog thereof.

In some embodiments, at least two luminescently labeled molecules absorb a first excitation energy, wherein at least one of the luminescently labeled molecules has a luminescent lifetime of less than about 1 ns, and at least one of the luminescently labeled molecules has a luminescent lifetime of greater than 1 ns. In some embodiments, at least two luminescently labeled molecules absorb a second excitation energy, wherein at least one of the luminescently labeled molecules has a luminescent lifetime of less than about 1 ns, and at least one of the luminescently labeled molecules has a luminescent lifetime of greater than 1 ns. In some embodiments, at least two luminescently labeled molecules absorb a first excitation energy, wherein at least one of the luminescently labeled molecules has a luminescent lifetime of less than about 1 ns, and at least one of the luminescently labeled molecules has a luminescent lifetime of greater than 1 ns, and at least additional two luminescently labeled molecules absorb a second excitation energy, wherein at least one of the luminescently labeled molecules has a luminescent lifetime of less than about 1 ns, and at least one of the luminescently labeled molecules has a luminescent lifetime of greater than 1 ns.

In some embodiments, at least two luminescently labeled amino acid recognition molecules (e.g., terminal amino acid recognizers) absorb a first excitation energy, wherein at least one of the labels comprises a cyanine dye, or analog thereof. In some embodiments, at least two luminescently labeled recognition molecules absorb a second excitation energy, wherein at least one of the labels comprises a cyanine dye, or analog thereof. In certain embodiments, the luminescent label is a dye selected from Table 3. The dyes listed in Table 3 are non-limiting, and the luminescent labels of the disclosure may include dyes not listed in Table 3. In certain embodiments, the luminescent labels of one or more luminescently labeled nucleotides or recognition molecules is selected from Table 3.

TABLE 3
Exemplary fluorophores.
Fluorophores

| | | |
|---|---|---|
| 5/6-Carboxyrhodamine 6G | Chromis 678C | DyLight ® 655-B1 |
| 5-Carboxyrhodamine 6G | Chromis 678Z | DyLight ® 655-B2 |
| 6-Carboxyrhodamine 6G | Chromis 770A | DyLight ® 655-B3 |
| 6-TAMRA | Chromis 770C | DyLight ® 655-B4 |
| Alexa Fluor ® 350 | Chromis 800A | DyLight ® 662Q |
| Alexa Fluor ® 405 | Chromis 800C | DyLight ® 675-B1 |
| Alexa Fluor ® 430 | Chromis 830A | DyLight ® 675-B2 |
| Alexa Fluor ® 480 | Chromis 830C | DyLight ® 675-B3 |
| Alexa Fluor ® 488 | Cy ® 3 | DyLight ® 675-B4 |
| Alexa Fluor ® 514 | Cy ® 3.5 | DyLight ® 679-C5 |
| Alexa Fluor ® 532 | Cy ® 3B | DyLight ® 680 |
| Alexa Fluor ® 546 | Cy ® 5 | DyLight ® 683Q |
| Alexa Fluor ® 555 | Dyomics-350 | DyLight ® 690-B1 |
| Alexa Fluor ® 568 | Dyomics-350XL | DyLight ® 690-B2 |
| Alexa Fluor ® 594 | Dyomics-360XL | DyLight ® 696Q |
| Alexa Fluor ® 610-X | Dyomics-370XL | DyLight ® 700-B1 |
| Alexa Fluor ® 633 | Dyomics-375XL | DyLight ® 700-B1 |
| Alexa Fluor ® 647 | Dyomics-380XL | DyLight ® 730-B1 |
| Alexa Fluor ® 660 | Dyomics-390XL | DyLight ® 730-B2 |
| Alexa Fluor ® 680 | Dyomics-405 | DyLight ® 730-B3 |
| Alexa Fluor ® 700 | Dyomics-415 | DyLight ® 730-B4 |
| Alexa Fluor ® 750 | Dyomics-430 | DyLight ® 747 |
| Alexa Fluor ® 790 | Dyomics-431 | DyLight ® 747-B1 |
| AMCA | Dyomics-478 | DyLight ® 747-B2 |
| ATTO 390 | Dyomics-480XL | DyLight ® 747-B3 |
| ATTO 425 | Dyomics-481XL | DyLight ® 747-B4 |
| ATTO 465 | Dyomics-485XL | DyLight ® 755 |
| ATTO 488 | Dyomics-490 | DyLight ® 766Q |
| ATTO 495 | Dyomics-495 | DyLight ® 775-B2 |
| ATTO 514 | Dyomics-505 | DyLight ® 775-B3 |
| ATTO 520 | Dyomics-510XL | DyLight ® 775-B4 |
| ATTO 532 | Dyomics-511XL | DyLight ® 780-B1 |
| ATTO 542 | Dyomics-520XL | DyLight ® 780-B2 |
| ATTO 550 | Dyomics-521XL | DyLight ® 780-B3 |
| ATTO 565 | Dyomics-530 | DyLight ® 800 |
| ATTO 590 | Dyomics-547 | DyLight ® 830-B2 |
| ATTO 610 | Dyomics-547P1 | eFluor ® 450 |
| ATTO 620 | Dyomics-548 | Eosin |
| ATTO 633 | Dyomics-549 | FITC |
| ATTO 647 | Dyomics-549P1 | Fluorescein |
| ATTO 647N | Dyomics-550 | HiLyte ™ Fluor 405 |
| ATTO 655 | Dyomics-554 | HiLyte ™ Fluor 488 |
| ATTO 665 | Dyomics-555 | HiLyte ™ Fluor 532 |
| ATTO 680 | Dyomics-556 | HiLyte ™ Fluor 555 |
| ATTO 700 | Dyomics-560 | HiLyte ™ Fluor 594 |
| ATTO 725 | Dyomics-590 | HiLyte ™ Fluor 647 |
| ATTO 740 | Dyomics-591 | HiLyte ™ Fluor 680 |
| ATTO Oxa12 | Dyomics-594 | HiLyte ™ Fluor 750 |
| ATTO Rho101 | Dyomics-601XL | IRDye ® 680LT |
| ATTO Rho11 | Dyomics-605 | IRDye ® 750 |
| ATTO Rho12 | Dyomics-610 | IRDye ® 800CW |
| ATTO Rho13 | Dyomics-615 | JOE |
| ATTO Rho14 | Dyomics-630 | LightCycler ® 640R |
| ATTO Rho3B | Dyomics-631 | LightCycler ® Red 610 |
| ATTO Rho6G | Dyomics-632 | LightCycler ® Red 640 |
| ATTO Thio12 | Dyomics-633 | LightCycler ® Red 670 |
| BD Horizon ™ V450 | Dyomics-634 | LightCycler ® Red 705 |
| BODIPY ® 493/501 | Dyomics-635 | Lissamine Rhoda- |
| | | mine B |
| BODIPY ® 530/550 | Dyomics-636 | Napthofluorescein |
| BODIPY ® 558/568 | Dyomics-647 | Oregon Green ® 488 |
| BODIPY ® 564/570 | Dyomics-647P1 | Oregon Green ® 514 |
| BODIPY ® 576/589 | Dyomics-648 | Pacific Blue ™ |
| BODIPY ® 581/591 | Dyomics-648P1 | Pacific Green ™ |
| BODIPY ® 630/650 | Dyomics-649 | Pacific Orange ™ |
| BODIPY ® 650/665 | Dyomics-649P1 | PET |
| BODIPY ® FL | Dyomics-650 | PF350 |
| BODIPY ® FL-X | Dyomics-651 | PF405 |
| BODIPY ® R6G | Dyomics-652 | PF415 |
| BODIPY ® TMR | Dyomics-654 | PF488 |
| BODIPY ® TR | Dyomics-675 | PF505 |
| C5.5 | Dyomics-676 | PF532 |
| C7 | Dyomics-677 | PF546 |
| CAL Fluor ® Gold 540 | Dyomics-678 | PF555P |
| CAL Fluor ® Green 510 | Dyomics-679P1 | PF568 |
| CAL Fluor ® Orange 560 | Dyomics-680 | PF594 |
| CAL Fluor ® Red 590 | Dyomics-681 | PF610 |
| CAL Fluor ® Red 610 | Dyomics-682 | PF633P |
| CAL Fluor ® Red 615 | Dyomics-700 | PF647P |
| CAL Fluor ® Red 635 | Dyomics-701 | Quasar ® 570 |
| Cascade ® Blue | Dyomics-703 | Quasar ® 670 |
| CF ™ 350 | Dyomics-704 | Quasar ® 705 |
| CF ™ 405M | Dyomics-730 | Rhoadmine 123 |
| CF ™ 405S | Dyomics-731 | Rhodamine 6G |
| CF ™ 488A | Dyomics-732 | Rhodamine B |
| CF ™ 514 | Dyomics-734 | Rhodamine Green |
| CF ™ 532 | Dyomics-749 | Rhodamine Green-X |
| CF ™ 543 | Dyomics-749P1 | Rhodamine Red |
| CF ™ 546 | Dyomics-750 | ROX |
| CF ™ 555 | Dyomics-751 | ROX |
| CF ™ 568 | Dyomics-752 | Seta ™ 375 |
| CF ™ 594 | Dyomics-754 | Seta ™ 470 |
| CF ™ 620R | Dyomics-776 | Seta ™ 555 |
| CF ™ 633 | Dyomics-777 | Seta ™ 632 |
| CF ™ 633-V1 | Dyomics-778 | Seta ™ 633 |
| CF ™ 640R | Dyomics-780 | Seta ™ 650 |
| CF ™ 640R-V1 | Dyomics-781 | Seta ™ 660 |
| CF ™ 640R-V2 | Dyomics-782 | Seta ™ 670 |
| CF ™ 660C | Dyomics-800 | Seta ™ 680 |
| CF ™ 660R | Dyomics-831 | Seta ™ 700 |
| CF ™ 680 | DyLight ® 350 | Seta ™ 750 |
| CF ™ 680R | DyLight ® 405 | Seta ™ 780 |
| CF ™ 680R-V1 | DyLight ® 415- | Seta ™ APC-780 |
| | Co1 | |
| CF ™ 750 | DyLight ® 425Q | Seta ™ PerCP-680 |
| CF ™ 770 | DyLight ® 485-LS | Seta ™ R-PE-670 |
| CF ™ 790 | DyLight ® 488 | Seta ™ 646 |
| Chromeo ™ 642 | DyLight ® 504Q | Seta ™ u 380 |
| Chromis 425N | DyLight ® 510-LS | Seta ™ u 425 |
| Chromis 500N | DyLight ® 515-LS | Seta ™ u 647 |
| Chromis 515N | DyLight ® 521-LS | Seta ™ u 405 |
| Chromis 530N | DyLight ® 530-R2 | Sulforhodamine 101 |
| Chromis 550A | DyLight ® 543Q | TAMRA |
| Chromis 550C | DyLight ® 550 | TET |
| Chromis 550Z | DyLight ® 554-R0 | Texas Red ® |
| Chromis 560N | DyLight ® 554-R1 | TMR |
| Chromis 570N | DyLight ® 590-R2 | TRITC |
| Chromis 577N | DyLight ® 594 | Yakima Yellow ™ |
| Chromis 600N | DyLight ® 610-B1 | Zenon ® |
| Chromis 630N | DyLight ® 615-B2 | Zy3 |
| Chromis 645A | DyLight ® 633 | Zy5 |
| Chromis 645C | DyLight ® 633-B1 | Zy5.5 |
| Chromis 645Z | DyLight ® 633-B2 | Zy7 |
| Chromis 678A | DyLight ® 650 | Abberior ® ® Star 635 |
| Square 635 | Square 650 | Square 660 |
| Square 672 | Square 680 | Abberior ® Star |
| | | 440SXP |
| Abberior ® Star 470SXP | Abberior ® Star | Abberior ® Star 512 |
| | 488 | |
| Abberior ® Star 520SXP | Abberior ® Star | Abberior ® Star 600 |
| | 580 | |
| Abberior ® Star 635 | Abberior ® Star | Abberior ® Star RED |
| | 635P | |

In certain embodiments, the luminescent label may comprise a first and second chromophore. In some embodiments, an excited state of the first chromophore is capable of relaxation via an energy transfer to the second chromophore. In some embodiments, the energy transfer is a Forster resonance energy transfer (FRET). Such a FRET pair may be useful for providing a luminescent label with properties that make the label easier to differentiate from amongst a plurality of luminescent labels. In certain embodiments, the FRET pair may absorb excitation energy in a first spectral range and emit luminescence in a second spectral range.

For a set of luminescently labeled molecules (e.g., luminescently labeled nucleotides or amino acid recognition molecules), the properties of a luminescently labeled FRET pair may allow for selection of a plurality of distinguishable molecules (e.g., nucleotides, amino acid recognition molecules). In some embodiments, the second chromophore of a FRET pair has a luminescent lifetime distinct from a plurality of other luminescently labeled molecules. In some embodiments, the second chromophore of a FRET pair has a luminescent intensity distinct from a plurality of other luminescently labeled molecules. In some embodiments, the second chromophore of a FRET pair has a luminescent lifetime and luminescent intensity distinct from a plurality of other luminescently labeled molecules. In some embodiments, the second chromophore of a FRET pair emits photons in a spectral range distinct from a plurality of other luminescently labeled molecules. In some embodiments, the first chromophore of a FRET pair has a luminescent lifetime distinct from a plurality of luminescently labeled molecules. In certain embodiments, the FRET pair may absorb excitation energy in a spectral range distinct from a plurality of other luminescently labeled molecules. In certain embodiments, the FRET pair may absorb excitation energy in the same spectral range as one or more of a plurality of other luminescently labeled molecules.

In some embodiments of the nucleic acid sequencing methods described herein, two or more nucleotides can be connected to a luminescent label, wherein the nucleotides are connected to distinct locations on the luminescent label. A non-limiting example could include a luminescent molecule that contains two independent reactive chemical moieties (e.g., azido group, acetylene group, carboxyl group, amino group) that are compatible with a reactive moiety on a nucleotide analog. In such an embodiment, a luminescent label could be connected to two nucleotide molecules via independent linkages. In some embodiments, a luminescent label can comprise two or more independent connections to two or more nucleotides.

In some embodiments, a quenching moiety is connected to one or more luminescent molecules and to one or more nucleotide molecules. As used herein, a nucleotide in this context refers to a nucleoside polyphosphate that can be incorporated into a growing nucleic acid, e.g., in the context of a sequencing reaction. In some embodiments, the luminescent molecule(s) are not adjacent to the nucleotide(s). For example, one or more luminescent molecules can be connected on a first side of the quenching moiety and one or more nucleotides can be connected to a second side of the quenching moiety, wherein the first and second sides of the quenching moiety are distant from each other. In some embodiments, they are on approximately opposite sides of the quenching moiety.

The distance between the point at which a quenching moiety is connected to a luminescent label and the point at which the quenching moiety is connected to a nucleotide can be a linear measurement through space or a non-linear measurement across the surface of the quenching moiety. The distance between the luminescent label and nucleotide connection points on a quenching moiety can be measured by modeling the three-dimensional structure of the quenching moiety. In some embodiments, this distance can be 2, 4, 6, 8, 10, 12, 14, 16, 18, 20 nm or more. Alternatively, the relative positions of the luminescent label and nucleotide on a quenching moiety can be described by treating the structure of the quenching moiety as a quadratic surface (e.g., ellipsoid, elliptic cylinder). In some embodiments, the luminescent label and the nucleotide are separated by a distance that is at least one eighth of the distance around an ellipsoidal shape representing the quenching moiety. In some embodiments, the luminescent label and the nucleotide are separated by a distance that is at least one quarter of the distance around an ellipsoidal shape representing the quenching moiety. In some embodiments, the luminescent label and the nucleotide are separated by a distance that is at least one third of the distance around an ellipsoidal shape representing the quenching moiety. In some embodiments, the luminescent label and the nucleotide are separated by a distance that is one half of the distance around an ellipsoidal shape representing the quenching moiety.

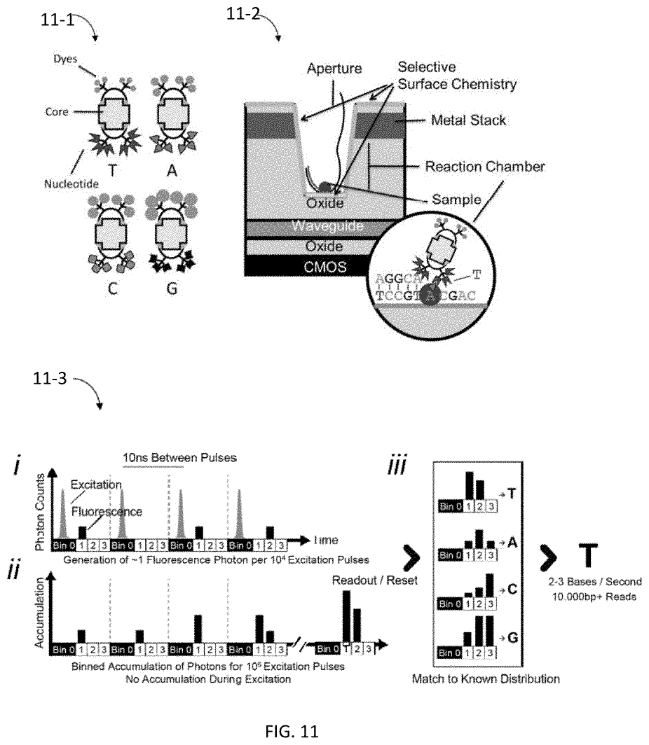

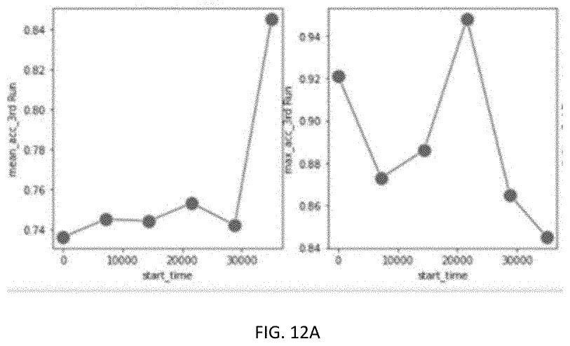

FIG. 11 illustrates a non-limiting example of a nucleic acid sequencing experiment and how unique luminescent properties can be used to distinguish among a plurality of luminescently labeled nucleotides 11-1. The luminescent label connected to each base (thymine, adenine, cytosine, guanine) has luminescent properties (e.g., luminescent lifetime, luminescent intensity, and/or emission wavelength) that allow each labeled nucleotide to be distinguished from the plurality of labeled nucleotides. The inclusion of multiple nucleotides of the same type functions to accelerate incorporation rates in a sequencing reaction.

A sequencing experiment utilizing the luminescently labeled nucleotides 11-1 can be conducted in exemplary reaction vessel 11-2. The reaction takes place in a chamber above the waveguide, which serves as a conduit for excitation energy, delivering the excitation energy to the sample in the bottom of the reaction chamber by the evanescent wave from the waveguide, such as a zero-mode waveguide. The aperture blocks light radiating from the waveguide to bulk sample and ambient and/or stray light from the sensor, as well as providing a fabrication path for the reaction chamber. The reaction chamber is an etched structure that places the sample on the bottom and within a region of high excitation from the evanescent wave of the waveguide. Selective surface chemistry is used to provide the bottom and sidewall of the reaction chamber with different composition, so that the sample can be selectively localized to the bottom of the reaction chamber.

The incorporation of a specific nucleotide can be distinguished from among four luminescently labeled nucleotides during a sequencing reaction per the exemplary workflow 11-3. Throughout the course of an experiment, there are two distinct periods: a pulse period and a detection period. During the pulse period, lasting 20 picoseconds, no emission light is collected. Following the pulse period is the detection period, lasting 10 nanoseconds, wherein four time bins capture emission events occurring over the detection period (i). A pulse and detection period comprise one cycle. Emission events are continuously binned and accumulated over the course of 1 million cycles (ii). The overall distribution of emission events across time bins are representative of luminescent lifetime and can be used to match a particular set of a data to a known lifetime distribution (iii). In some embodiments, the distribution of emission events (e.g., luminescent lifetime) does not distinguish one luminescently labeled base from a plurality of other labeled molecules. In addition to the distribution of emission events, the quantity of emission events (e.g., luminescent intensity) can be used to identify a single molecule from a plurality of others.

In some embodiments, the luminescent molecule and the nucleotide can be attached to the non-protein (e.g., nucleic acid) quenching moiety via reactive moieties. In this example, a reactive moiety on the luminescent molecule is covalently attached to the non-protein quenching moiety via a corresponding reactive moiety on the non-protein quenching moiety. A reactive moiety on the nucleotide is covalently attached to the non-protein quenching moiety via a corresponding reactive moiety on the non-protein quenching moiety. The non-protein quenching moiety may comprise, in some embodiments, a group selected from reactive amines, azides, alkynes, nitrones, alkenes (e.g., cycloalkenes), tetrazines, tetrazoles, and other reactive moieties suitable for click reactions and similar coupling techniques.

In some embodiments, one or more luminescent labels and/or one or more nucleotides (e.g., of the same type) can be attached at one or more different positions on a single-stranded or double-stranded oligonucleotide, including at one or more terminal and/or internal positions of the oligonucleotide.

In some embodiments, additional structural motifs (e.g., based on physical properties conferred by the sequence of the oligonucleotide quenching moiety, and/or based on one or more chemical modifications of one or more portions of the oligonucleotide quenching moiety) can be included in a single-stranded or double-stranded oligonucleotide quenching moiety, for example to increase the rigidity or to provide particular 3-dimensional configuration. In some embodiments, one or more hybridized regions (e.g., stem structures, or hybridized complementary regions of two oligonucleotides) can be stabilized (e.g., by covalent modification).

In some embodiments, one or more positions on an oligonucleotide quenching moiety can be modified to include one or more energy absorbing moieties. In some embodiments, one or more energy absorbing moieties can be added to the same nucleic acid strand that the label is attached to. In some embodiments, one or more energy absorbing moieties can be added to a different strand from the strand that is labeled in the context of a double-stranded oligonucleotide quenching moiety (e.g., the one or more energy absorbing moieties can be included on the complementary strand that is attached to one or more nucleotides).

### Detection/Quantitation of Molecules Using Luminescent Lifetimes

In some aspects of the methods described herein, an emitted photon (a luminescence) or a plurality of emitted photons is detected by one or more sensors. For a plurality of luminescently labeled molecules, each of the molecules may emit photons in a single spectral range, or a portion of the molecules may emit photons in a first spectral range and another portion of molecules may emit photons in a second spectral range. In certain embodiments, the emitted photons are detected by a single sensor. In certain embodiments, the emitted photons are detected by multiple sensors. In some embodiments, the photons emitted in a first spectral range are detected by a first sensor, and the photons emitted in a second spectral range are detected by a second sensor. In some embodiments, the photons emitted in each of a plurality of spectral ranges are detected by a different sensor.

In certain embodiments, each sensor is configured to assign a time bin to an emitted photon based on the time duration between the excitation energy and the emitted photon. In some embodiments, photons emitted after a shorter time duration will be assigned an earlier time bin, and photons emitted after a longer duration will be assigned a later time bin.

In some embodiments, a plurality of pulses of excitation energy is delivered to vicinity of a target volume and a plurality of photons, which may include photon emission events, are detected. In some embodiments, the plurality of luminescences (e.g., photon emission events) correspond to incorporation of a luminescently labeled nucleotide into a nucleic acid product. In some embodiments, the incorporation of a luminescently labeled nucleotide lasts for between about 1 ms and about 5 ms, between about 5 ms and about 20 ms, between about 20 ms and about 100 ms, or between about 100 ms and about 500 ms. In some embodiments, between about 10 and about 100, between about 100 and about 1000, about 1000 and about 10000, or about 10000 and about 100000 luminescences are detected during incorporation of a luminescently labeled nucleotide.

In certain embodiments, there are no luminescences detected if a luminescently labeled nucleotide is not being incorporated. In some embodiments, there is a luminescence background. In some embodiments, spurious luminescences are detected when no luminescently labeled nucleotide is being incorporated. Such spurious luminescences may occur if one or more luminescently labeled nucleotides is in the target volume (e.g., diffuses into the target volume, or interacts with a nucleic acid polymerase but is not incorporated) during a pulse of excitation energy, but is not being incorporated by the sequencing reaction. In some embodiments, the plurality of luminescences detected from a luminescently labeled nucleotide in the target volume but not being incorporated is smaller (e.g., ten times, 100 times, 1000 times, 10000 times) than the plurality of luminescences from a luminescently labeled nucleotide.

In some embodiments of the nucleic acid sequencing methods provided herein, for each plurality of detected luminescences corresponding to incorporation of a luminescently labeled nucleotide the luminescences are assigned a time bin based on the time duration between the pulse and the emitted photon. This plurality for an incorporation event is referred to herein as a “burst”. In some embodiments, a burst refers to a series of signals (e.g., measurements) above a baseline (e.g., noise threshold value), wherein the signals correspond to a plurality of emission events that occur when the luminescently labeled nucleotide is within the excitation region. In some embodiments, a burst is separated from a preceding and/or subsequent burst by a time interval of signals representative of the baseline. In some embodiments, the burst is analyzed by determining the luminescent lifetime based on the plurality of time durations. In some embodiments, the burst is analyzed by determining the luminescent intensity based on the number of detected luminescences per a unit of time. In some embodiments, the burst is analyzed by determining the spectral range of the detected luminescences. In some embodiments, analyzing the burst data will allow assignment of the identity of the incorporated luminescently labeled nucleotide, or allow one or more luminescently labeled nucleotides to be differentiated from amongst a plurality of luminescently labeled nucleotides. The assignment or differentiation may rely on any one of luminescent lifetime, luminescent intensity, spectral range of the emitted photons, or any combination thereof.

An integrated circuit having an integrated photodetector according to aspects of the present application may be designed with suitable functions for a variety of detection and imaging applications. As described in further detail below, such an integrated photodetector can have the ability to detect light within one or more time intervals, or “time bins.” To collect information regarding the time of arrival of the light, charge carriers are generated in response to incident photons and can be segregated into respective time bins based upon their time of arrival.

In some embodiments, an integrated photodetector as described herein can detect the luminescent characteristics of labeled biological and/or chemical sample(s) in response to excitation. More specifically, such an integrated photodetector can detect the temporal characteristics of light received from the labeled sample(s). Such an integrated photodetector can enable detecting and/or discriminating the luminescent lifetime of light emitted by a luminescent molecule in response to excitation. In some embodiments, identification and/or quantitative measurements of sample(s) can be performed based on detecting and/or discriminating luminescent lifetimes. For example, in some embodiments sequencing of a nucleic acid (e.g., DNA, RNA) may be performed by detecting and/or discriminating luminescent lifetimes of luminescent molecules attached to respective nucleotides.

In some embodiments, an integrated photodetector having a number of photodetection structures and associated electronics, termed “pixels,” can enable measurement and analysis of a plurality of samples in parallel (e.g., hundreds, thousands, millions or more), which can reduce the cost of performing complex measurements and rapidly advance the rate of discoveries. In some embodiments, each pixel of the photodetector may detect light from a sample, which may be a single molecule or more than one molecule. In some embodiments, such an integrated photodetector can be used for dynamic real time applications such as nucleic acid (e.g., DNA, RNA) sequencing.

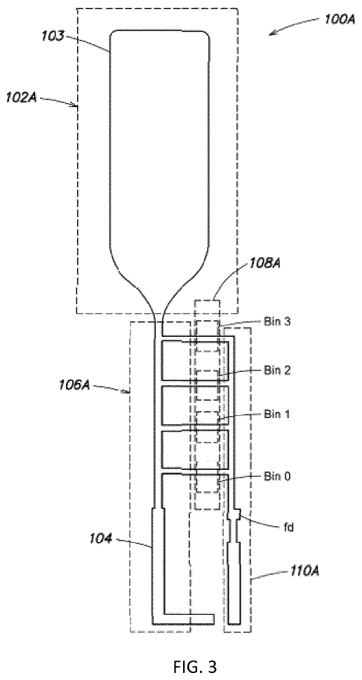

An exemplary charge carrier confinement region of a pixel is depicted in the schematic of FIG. 3. As illustrated in this figure, pixel 100A may include a photon absorption/carrier generation area 102A (also referred to as a detection region), a carrier travel/capture area 106A, a drain 104, a plurality of charge carrier storage bins bin0, bin1, bin2, and bin3 of a carrier storage region 108A, and a readout circuitry 110A for reading out signals from the charge carrier storage bins.

Charge carrier confinement region 103 is a region in which photogenerated charge carriers move in response to the electric potential gradient produced by a charge carrier segregation structure. Charge carriers may be generated in photon absorption/carrier generation area 102A within charge carrier confinement region 103.

The charge carrier confinement regions of the disclosure may be formed of any suitable material, such as a semiconductor material (e.g., silicon). However, the methods described herein are not limited in this respect, as any suitable material may form a charge carrier confinement region. In some embodiments, a charge carrier confinement region may be surrounded by an insulator (e.g., silicon oxide) to confine charge carriers within the confinement region.

As shown in FIG. 3, a first portion of charge carrier confinement region 103 in carrier travel/capture area 106A may extend from the photon absorption/carrier generation area 102A to a drain 104. Extensions of the charge carrier confinement region 103 extend to the respective charge storage bins, allowing charge carriers to be directed into the charge carrier storage bins by a charge carrier segregation structure such as that described with respect to FIG. 3. In some embodiments, the number of extensions of the charge carrier confinement region 103 that are present may be the same as the number of charge carrier storage bins, with each extension extending to a respective charge carrier storage bin.

An integrated photodetector according to some aspects of the present application may be used for differentiating among light emission sources, including luminescent molecules, such as fluorophores. Luminescent molecules vary in the wavelength of light they emit, the temporal characteristics of the light they emit (e.g., their emission decay time periods), and their response to excitation energy. Accordingly, luminescent molecules may be identified or discriminated from other luminescent molecules based on detecting these properties. Such identification or discrimination techniques may be used alone or in any suitable combination.

In some embodiments, an integrated photodetector as described in the present application can measure or discriminate luminescent lifetimes. Luminescent lifetime measurements are based on exciting one or more fluorescent molecules, and measuring the time variation in the emitted luminescence. The probability of a fluorescent molecule to emit a photon after the fluorescent molecule reaches an excited state decreases exponentially over time. The rate at which the probability decreases may be characteristic of a fluorescent molecule, and may be different for different fluorescent molecules. Detecting the temporal characteristics of light emitted by fluorescent molecules may allow identifying fluorescent molecules and/or discriminating fluorescent molecules with respect to one another. Luminescent molecules are also referred to herein as luminescent markers (or simply “markers”), labels and fluorophores.

After reaching an excited state, a marker may emit a photon with a certain probability at a given time. The probability of a photon being emitted from an excited marker may decrease over time after excitation of the marker. The decrease in the probability of a photon being emitted over time may be represented by an exponential decay function p(t)=e^{-t/τ}, where p(t) is the probability of photon emission at a time, t, and τ is a temporal parameter of the marker. The temporal parameter i indicates a time after excitation when the probability of the marker emitting a photon is a certain value. The temporal parameter, τ, is a property of a marker that may be distinct from its absorption and emission spectral properties. Such a temporal parameter, τ, is referred to as the luminescent lifetime, or simply the “lifetime”, of a marker. Markers may have luminescent lifetimes ranging from 0.1-20 ns, in some embodiments. However, the techniques described herein are not limited as to the lifetimes of the marker(s) used.

The lifetime of a marker may be used to distinguish among more than one marker, and/or may be used to identify marker(s). In some embodiments, luminescent lifetime measurements may be performed in which a plurality of markers having different lifetimes are excited by an excitation source. As an example, four markers having lifetimes of 0.5, 1, 2, and 3 nanoseconds, respectively, may be excited by a light source that emits light having a selected wavelength (e.g., 635 nm, by way of example). The markers may be identified or differentiated from each other based on measuring the lifetime of the light emitted by the markers.

Luminescent lifetime measurements may use relative intensity measurements by comparing how intensity changes over time, as opposed to absolute intensity values. As a result, luminescent lifetime measurements may avoid some of the difficulties of absolute intensity measurements. Absolute intensity measurements may depend on the concentration of fluorophores present and calibration steps may be needed for varying fluorophore concentrations. By contrast, luminescent lifetime measurements may be insensitive to the concentration of fluorophores.

Differentiating between markers by lifetime measurements may allow for fewer wavelengths of excitation light to be used than when the markers are differentiated by measurements of emission spectra. In some embodiments, sensors, filters, and/or diffractive optics may be reduced in number or eliminated when using fewer wavelengths of excitation light and/or luminescent light. In some embodiments, labeling may be performed with markers that have different lifetimes, and the markers may be excited by light having the same excitation wavelength or spectrum. In some embodiments, an excitation light source may be used that emits light of a single wavelength or spectrum, which may reduce the cost. However, the techniques described herein are not limited in this respect, as any number of excitation light wavelengths or spectra may be used. In some embodiments, an integrated photodetector may be used to determine both spectral and temporal information regarding received light. In some embodiments a quantitative analysis of the types of molecule(s) present may be performed by determining a temporal parameter, a spectral parameter, or a combination of the temporal and spectral parameters of the emitted luminescence from a marker.

An integrated photodetector that detects the arrival time of incident photons may reduce additional optical filtering (e.g., optical spectral filtering) requirements. As described below, an integrated photodetector according to the present application may include a drain to remove photogenerated carriers at particular times. By removing photogenerated carriers in this manner, unwanted charge carriers produced in response to an excitation light pulse may be discarded without the need for optical filtering to prevent reception of light from the excitation pulse. Such a photodetector may reduce overall design integration complexity, optical and/or filtering components, and/or cost.

In some embodiments, a luminescent lifetime may be determined by measuring the time profile of the emitted luminescence by aggregating collected charge carriers in one or more time bins of the integrated photodetector to detect luminescent intensity values as a function of time. In some embodiments, the lifetime of a marker may be determined by performing multiple measurements where the marker is excited into an excited state and then the time when a photon emits is measured. For each measurement, the excitation source may generate a pulse of excitation light directed to the marker, and the time between the excitation pulse and subsequent photon event from the marker may be determined. Additionally or alternatively, when an excitation pulse occurs repeatedly and periodically, the time between when a photon emission event occurs and the subsequent excitation pulse may be measured, and the measured time may be subtracted from the time interval between excitation pulses (i.e., the period of the excitation pulse waveform) to determine the time of the photon absorption event.

By repeating such experiments with a plurality of excitation pulses, the number of instances a photon is emitted from the marker within a certain time interval after excitation may be determined, which is indicative of the probability of a photon being emitted within such a time interval after excitation. The number of photon emission events collected may be based on the number of excitation pulses emitted to the marker. The number of photon emission events over a measurement period may range from 50-10,000,000 or more, in some embodiments, however, the techniques described herein are not limited in this respect. The number of instances a photon is emitted from the marker within a certain time interval after excitation may populate a histogram representing the number of photon emission events that occur within a series of discrete time intervals or time bins. The number of time bins and/or the time interval of each bin may be set and/or adjusted to identify a particular lifetime and/or a particular marker. The number of time bins and/or the time interval of each bin may depend on the sensor used to detect the photons emitted. The number of time bins may be 1, 2, 3, 4, 5, 6, 7, 8, or more, such as 16, 32, 64, or more. A curve fitting algorithm may be used to fit a curve to the recorded histogram, resulting in a function representing the probability of a photon to be emitted after excitation of the marker at a given time. An exponential decay function, such as p(t)=e^{-t/τ}, may be used to approximately fit the histogram data. From such a curve fitting, the temporal parameter or lifetime may be determined. The determined lifetime may be compared to known lifetimes of markers to identify the type of marker present.

A lifetime may be calculated from the intensity values at two time intervals. In some embodiments, the photodetector measures the intensity over at least two time bins. The photons that emit luminescence energy between times t1 and t2 are measured by the photodetector as intensity I1 and luminescence energy emitted between times t3 and t4 are measured as I2. Any suitable number of intensity values may be obtained. Such intensity measurements may then be used to calculate a lifetime. When one fluorophore is present at a time, then the time binned luminescence signal may be fit to a single exponential decay. In some embodiments, only two time bins may be needed to accurately identify the lifetime for a fluorophore. When two or more fluorophores are present, then individual lifetimes may be identified from a combined luminescence signal by fitting the luminescence signal to multiple exponential decays, such as double or triple exponentials. In some embodiments two or more time bins may be needed in order to accurately identify more than one luminescent lifetime from such a signal. However, in some instances with multiple fluorophores, an average luminescent lifetime may be determined by fitting a single exponential decay to the luminescence signal.

In some instances, the probability of a photon emission event and thus the lifetime of a marker may change based on the surroundings and/or conditions of the marker. For example, the lifetime of a marker confined in a volume with a diameter less than the wavelength of the excitation light may be smaller than when the marker is not in the volume. Lifetime measurements with known markers under conditions similar to when the markers are used for labeling may be performed. The lifetimes determined from such measurements with known markers may be used when identifying a marker.

### Sequencing Using Luminescent Lifetime Measurements

Individual pixels on an integrated photodetector may be capable of luminescent lifetime measurements used to identify fluorophores and/or reporters that label one or more targets, such as molecules or specific locations on molecules. Any one or more molecules of interest may be labeled with a fluorophore, including proteins, amino acids, enzymes, lipids, nucleotides, DNA, and RNA. When combined with detecting spectra of the emitted light or other labeling techniques, luminescent lifetime may increase the total number of fluorophores and/or reporters that can be used. Identification based on lifetime may be used for single molecule analytical methods to provide information about characteristics of molecular interactions in complex mixtures where such information would be lost in ensemble averaging and may include protein-protein interactions, enzymatic activity, molecular dynamics, and/or diffusion on membranes. Additionally, fluorophores with different luminescent lifetimes may be used to tag target components in various assay methods that are based on presence of a labeled component. In some embodiments, components may be separated, such as by using microfluidic systems, based on detecting particular lifetimes of fluorophores.

Measuring luminescent lifetimes may be used in combination with other analytical methods. For an example, luminescent lifetimes may be used in combination with fluorescence resonance energy transfer (FRET) techniques to discriminate between the states and/or environments of donor and acceptor fluorophores located on one or more molecules. Such measurements may be used to determine the distance between the donor and the acceptor. In some instances, energy transfer from the donor to the acceptor may decrease the lifetime of the donor. In another example, luminescent lifetime measurements may be used in combination with DNA sequencing techniques where four fluorophores having different lifetimes may be used to label the four different nucleotides (A, T, G, C) in a DNA molecule with an unknown sequence of nucleotides. The luminescent lifetimes, instead of emission spectra, of the fluorophores may be used to identify the sequence of nucleotides. By using luminescent lifetime instead of emission spectra for certain techniques, accuracy and measurement resolution may increase because artifacts due to absolute intensity measurements are reduced. Additionally, lifetime measurements may reduce the complexity and/or expense of the system because fewer excitation energy wavelengths are required and/or fewer emission energy wavelengths need be detected.

### EXAMPLES

### Example 1: On-Chip Evaluations in a DNA Sequencing Application

An array of sample wells on a CMOS chip was functionalized with biotin and azide coupling moieties. A sample containing a DNA molecule of interest was loaded into the sample wells. Subsequently, a graft copolymer of PLL-PEG comprising a trolox quenching moiety was added to the sample well, and biotin coupling moieties were conjugated to the copolymer in a click chemistry reaction, in accordance with the workflow illustrated in FIG. 8. A DNA polymerase was added to the sample well, and sequencing by synthesis allowed to commence.

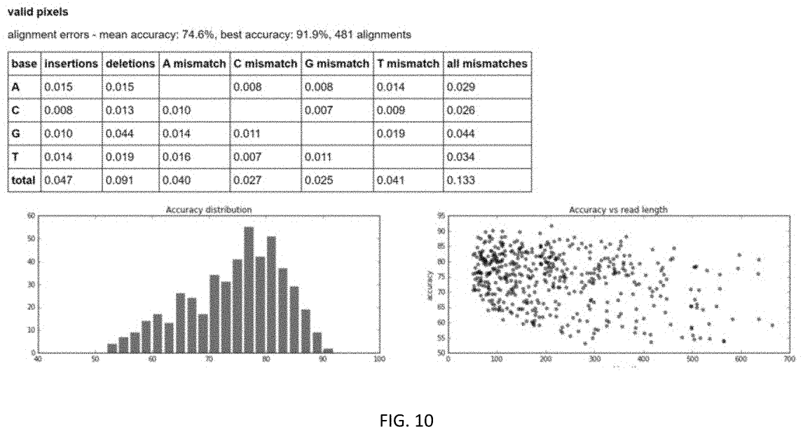

As shown in FIG. 10, an average (or mean) accuracy of 74.6% was observed in end nucleic acid sequencing performance, and a “best” accuracy of 91.9% was observed.

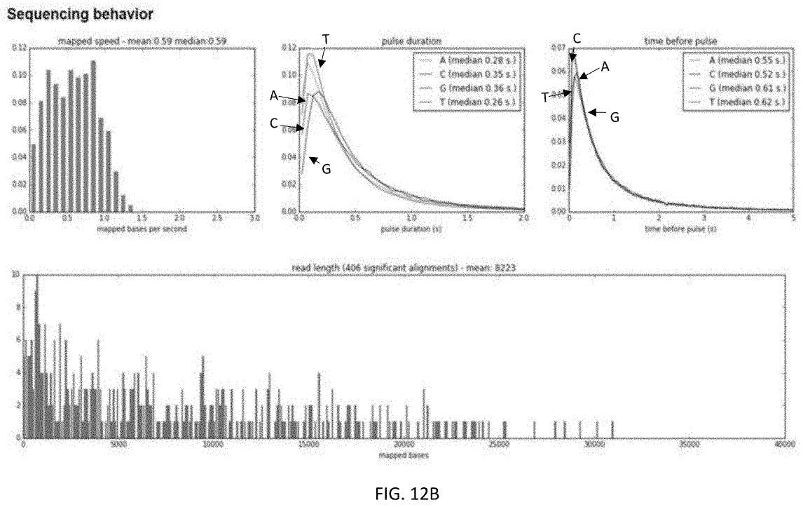

This workflow was repeated for several other coupling moiety-copolymer combinations (with streptavidin-biotin conjugates). In one such workflow, a graft copolymer of PLL-PEG comprising a cyclooctatetraene (COT) quenching moiety was added to the sample well, and biotin coupling moieties were conjugated to the copolymer in a click chemistry reaction. As shown in FIG. 12B, an average accuracy of 74% was observed over a 10 hour end sequencing experiment. A “best” accuracy of over 85% was observed over this 10 hour experiment. Each of these values were achieved over a mean read length (“RL”) of 8223 base pairs (bp).

As demonstrated in the results shown in FIGS. 10 and 12A-12B, the accuracy and read length results of DNA molecules in the presence of graft copolymers containing an exemplary quenching moiety as described herein significantly exceeded expectations. These values represent the highest average accuracy known to the inventors in the history of on-chip sequencing.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A method of sequencing a biomolecule in a sample, the method comprising:
functionalizing a bottom surface of a sample well with a coupling moiety;
coupling the coupling moiety with a copolymer comprising a triplet state quenching moiety;
contacting the sample well with the sample and a plurality of luminescently labeled molecules;
applying an excitation signal;
determining a luminescent lifetime, a retention time, a luminescent intensity, a luminescent wavelength, a pulse duration, and/or an interpulse duration of a luminescently labeled molecule that interacts with the biomolecule, wherein the luminescently labeled molecule that interacts with the biomolecule is one of the plurality of luminescently labeled molecules; and
identifying the luminescently labeled molecule that interacts with the biomolecule based on the luminescent lifetime, the retention time, the luminescent intensity, the luminescent wavelength, the pulse duration, and/or the interpulse duration of the luminescently labeled molecule.

2. The method of claim 1, wherein the coupling moiety comprises a biotin moiety, an avidin protein, a streptavidin protein, a biotin-streptavidin complex, an azide moiety, an alkyne moiety, a ketone moiety, or a hydroxylamine moiety.

3. The method of claim 1, wherein the coupling moiety comprises a biotin moiety.

4. (canceled)

5. The method of claim 1, wherein the copolymer comprises a click chemistry handle.

6. The method of claim 1, wherein the copolymer comprises a graft of poly-L-lysine (PLL) and polyethylene glycol (PEG).

7. The method of claim 1, wherein the triplet state quenching moiety is selected from the group consisting of trolox, trolox quinone (TQ), 4-nitrobenzyl alcohol (NBA), and cyclooctatetraene (COT).

8. The method of claim 1, wherein the triplet state quenching moiety comprises trolox or trolox quinone (TQ).

9. The method of claim 1, wherein the quenching moiety remains substantially immobilized in a target volume.

10. The method of claim 1, wherein the biomolecule is a polynucleotide.

11. (canceled)

12. The method of claim 1, wherein the luminescently labeled molecule that interacts with the biomolecule is a luminescently labeled nucleotide.

13. The method of claim 1, wherein the biomolecule is a polypeptide.

14. (canceled)

15. The method of claim 1, wherein the luminescently labeled molecule is an amino acid recognition molecule.

16. The method of claim 1 further comprising functionalizing the bottom surface of the sample well with a second coupling moiety, whereby the biomolecule binds the second coupling moiety.

17. The method of claim 16, wherein the second coupling moiety comprises a biotin moiety, an avidin protein, a streptavidin protein, an azide moiety, an alkyne moiety, a ketone moiety, a hydroxylamine moiety.

18. The method of claim 16, wherein the second coupling moiety comprises a biotin moiety.

19-20. (canceled)

21. An integrated device comprising:
a substrate comprising an array of sample wells having a metal oxide surface;
a functionalizing agent bound to the metal oxide surface, wherein the functionalizing agent comprises a coupling moiety; and
a copolymer comprising a triplet state quenching moiety;
wherein the coupling moiety comprises a biotin moiety, an avidin protein, a streptavidin protein, a biotin-streptavidin complex, an azide moiety, an alkyne moiety, a ketone moiety, or a hydroxylamine moiety.

22. The integrated device of claim 21, wherein the integrated device is configured to interface with a next-generation sequencing instrument.

22. (canceled)

23. The integrated device of claim 21, wherein the copolymer comprises a graft of poly-L-lysine (PLL) and polyethylene glycol (PEG).

24. The integrated device of claim 21, wherein the triplet state quenching moiety is selected from the group consisting of trolox, trolox quinone (TQ), 4-nitrobenzyl alcohol (NBA), and cyclooctatetraene (COT).

25. (canceled)

26. The integrated device of claim 21, wherein the copolymer comprises a click chemistry handle.
